(19) United States
(12) Patent Application Publication
(10) Pub. No.: US 2022/0016211 A1
(43) Pub. Date: Jan. 20, 2022

(54) FGF-21 CONJUGATE FORMULATIONS

(71) Applicant: Bristol-Myers Squibb Company, Princeton, NJ (US)

(72) Inventors: Thomas PALM, Helmetta, NJ (US); Mehrnaz KHOSSRAVI, West Windsor, NJ (US); Sanket PATKE, Belmont, MA (US)

(73) Assignee: Bristol-Myers Squibb Company, Princeton, NJ (US)

(21) Appl. No.: 17/143,938

(22) Filed: Jan. 7, 2021

Related U.S. Application Data
(60) Provisional application No. 62/958,580, filed on Jan. 8, 2020.

(51) Int. Cl. A61K 38/18; A61K 47/54; A61K 47/60; A61K 47/18; A61K 47/26

(52) U.S. Cl. CPC A61K 38/1825; A61K 47/547; A61K 47/26; A61K 47/183; A61K 47/60

(57) ABSTRACT

The present application provides pharmaceutical formulations comprising PEGylated FGF-21, e.g., a FGF-21 conjugate, and one or more stabilizers such as the chelator DPTA. The formulations can be can further stabilized by including a surfactant such as polysorbate 80 and/or adjusting the pH to about 7.1. Also provided are methods of manufacture, methods of treatment, and kits.

### REFERENCE TO SEQUENCE LISTING SUBMITTED ELECTRONICALLY VIA EFS-WEB

The content of the electronically submitted sequence listing (Name: 3338_2080002_SeqListing.txt; Size: 11,049 bytes; and Date of Creation: Jan. 5, 2021) filed with the application is incorporated herein by reference in its entirety.

### FIELD

This application pertains to, among other things, formulations comprising a FGF-21 polypeptide, e.g., a PEGylated FGF-21 polypeptide (PEG-FGF-21), stabilized using chelators (e.g., DTPA), surfactants (e.g., PS80), and having specific pH ranges (e.g., about 7.1), for administration via various routes, including, for example, subcutaneous administration.

### BACKGROUND

Fibroblast growth factors (FGF) are polypeptides widely expressed in developing and adult tissues (Baird et al., Cancer Cells, 3:239-243, 1991) that play crucial roles in multiple physiological functions (McKeehan et al., Prog. Nucleic Acid Res. Mol. Biol. 59:135-176, 1998; Burgess, W. H. et al., Annu. Rev. Biochem. 58:575-606 (1989). According to the literature, the FGF family consists of at least twenty-two members (Reuss et al., Cell Tissue Res. 313:139-157 (2003)).

FGF-21 can be used for the treatment of diseases and conditions associated with fibrosis. Fibrosis is the formation of excess fibrous connective tissue in an organ or tissue. Excess deposition of fibrous tissue is associated with pathological conditions that can lead to impairment of organ or tissue function. Affected organs can include the lungs (lung or pulmonary fibrosis), liver (liver or hepatic fibrosis), kidney (kidney or renal fibrosis), and heart (cardiac fibrosis). Fibrosis can also affect other tissues and organs including joints, skin, intestine, bone marrow, and others. Exemplary fibrotic conditions or diseases include, but are not limited to, nonalcoholic steatohepatitis (NASH), which affects the liver; diabetic kidney disease and diabetic nephropathy, which affect the kidney; and metabolic heart failure, which affects the heart. For example, NASH is characterized by fat accumulation, inflammation and damage in the liver of people who consume little or no alcohol, and the disease can lead to liver cirrhosis. NASH tends to be diagnosed in overweight or obese middle-aged people who often have elevated blood levels of certain lipids and diabetes or prediabetes.

FGF-21 has been reported to have a propensity to undergo proteolysis in vivo, form aggregates in vitro, and undergo deamidation (Gimeno and Moller, Trends Endocrinol Metab. 2014 June; 25(6):303-11; U.S. Pat. No. 8,361,963; Hecht et al., PLoS One. 2012; 7(11):e49345; U.S. Patent Publication No. 2007/0293430; WO 2006/0065582), potentially limiting the shelf-life of pharmaceutical compositions containing FGF-21. Aggregates and deamidated forms of therapeutic polypeptides may potentially increase immunogenicity (see U.S. Department of Health and Human Services, “Immunogenicity Assessment for Therapeutic Protein Products,” August 2014; Subramanyam (ed.), “Therapeutic Protein Immunogenicity Focus Group Newsletter,” American Association of Pharmaceutical Scientists, Vol. 1, Issue 3 (December 2011)).

The present disclosure addresses, among other things, the need for pharmaceutical formulations that address the problems associated with the production of pharmaceutical formulations comprising FGF-21 polypeptides, and in particular, variant FGF-21 polypeptides.

### BRIEF SUMMARY

The present disclosure provides a pharmaceutical formulation comprising (i) a fibroblast growth factor 21 (FGF-21) polypeptide conjugated to a polyethylene glycol (PEG) moiety (“FGF-21 conjugate”) and (ii) an aminopolycarboxylic acid cation chelator, wherein the formulation has improved stability compared to a reference formulation that does not contain the aminopolycarboxylic acid cation chelator, wherein the FGF-21 conjugate comprises formula I:

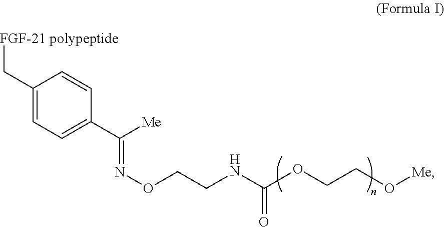

and wherein n is any integer.

In some aspects, the PEG moiety is conjugated to a non-natural amino acid in the FGF-21 polypeptide. In some aspects, the non-natural amino acid in the FGF-21 polypeptide is a phenylalanine derivative. In some aspects, the phenylalanine derivative is para-acetyl-L-phenylalanine.

In some aspects, the FGF-21 polypeptide comprises an amino acid sequence having at least about 80%, at least about 85%, at least about 90%, at least about 95%, at least about 96%, at least about 97%, at least about 98%, or at least about 99% amino acid sequence identity to the amino acid sequence of SEQ ID NO: 3, wherein the polypeptide has a FGF-21 activity. In some aspects, the non-natural amino acid is at amino acid residue 109 corresponding to SEQ ID NO: 3. In some aspects, the FGF-21 polypeptide comprises the sequence as set forth in SEQ ID NO: 1. In some aspects, the FGF-21 conjugate corresponds to a compound of SEQ ID NO: 2.

In some aspects, the n in formula I is from about 500 to about 900 ethylene glycol units, from about 600 to about 800 ethylene glycol units, from about 650 to about 750 ethylene glycol units, or from about 670 to about 690 ethylene glycol units. In some aspects, the n is between about 670 and about 690 ethylene glycol units, e.g., about 681 ethylene glycol units.

In some aspects, the FGF-21 conjugate corresponds to a compound of SEQ ID NO: 4. In some aspects, the FGF-21 conjugate is in an L conformation. In some aspects, the FGF-21 conjugate is in a D conformation. In some aspects, the FGF-21 conjugate is present at a concentration between about 1 mg/ml and about 40 mg/ml. In some aspects, the FGF-21 conjugate is present at a concentration of about 10 mg/ml or about 20 mg/ml. In some aspects, the FGF-21 conjugate is present at a concentration of about 20 mg/ml.

In some aspects, the FGF-21 conjugate is present in an amount between about 1 mg and about 40 mg per unit dose. In some aspects, the FGF-21 conjugate is present in an amount of about 1 mg per unit dose, about 5 mg per unit dose, about 10 mg per unit dose, about 20 mg per unit dose, or about 40 mg per unit dose. In some aspects, the FGF-21 conjugate is present in an amount of about 10 mg or about 20 mg per unit dose.

In some aspects, the pharmaceutical formulation exhibits one or more of:

(a) a lower rate of polypeptide deamidation when stored at 40° C. for about a month with respect to the reference formulation;
(b) a lower rate of high molecular weight (HMW) polypeptide aggregation when stored at 40° C. for about a month with respect to the reference formulation; or
(c) both (a) and (b).

In some aspects, the aminopolycarboxylic acid cation chelator prevents or mitigates oxidation of one or more methionines. In some aspects, the methionines correspond to Methionine 1 (Met1) and/or Methionine 169 (Met169) of the FGF-21 polypeptide. In some aspects, methionine oxidation is prevented or mitigated by the chelator at 25° C. and/or 40° C. In some aspects, the aminopolycarboxylic acid cation chelator is diethylenetriaminepentaacetic acid (DTPA). In some aspects, the DTPA cation chelator is present in an amount between about 10 μM and about 100 μM DTPA, between about 20 μM and about 90 μM DTPA, between about 30 μM and about 80 μM DTPA, between about 25 μM and about 75 μM DTPA, or between about 40 μM and about 60 μM DTPA, or between about 30 μM and about 70 μM DTPA, or between about 40 μM and about 70 μM DTPA. In some aspects, the DTPA cation chelator is present in an amount of about 40 μM, about 45 μM, about 50 μM, about 55 μM, or about 60 μM DTPA.

In some aspects, the pH of the formulation is above 6.5, above 6.6, above 6.7, above 6.8, above 6.9, above 7.0, above 7.1, above 7.2, above 7.3, above 7.4, or above 7.5. In some aspects, the pH is between about 6.7 and about 7.5, about 6.8 and about 7.5, about 6.9 and about 7.4, about 7.0 and about 7.3, about 7.1 and 7.2, about 7.1 and about 7.3, about 7.1 and about 7.4, or about 7.1 and about 7.5. In some aspects, the pH is about 6.7, about 6.8, about 6.9, about 7.0, about 7.1, about 7.2, about 7.3, about 7.4, or about 7.5. In some aspects, the pharmaceutical formulation is more stable than a reference formulation with a pH of 6.5.

In some aspects, the pharmaceutical formulation further comprises a surfactant. In some aspects, the surfactant is a nonionic surfactant. In some aspects, the nonanionic surfactant is a polysorbate. In some aspects, the polysorbate is polyoxyethylene (20) sorbitan monooleate (polysorbate 80). In some aspects, the polysorbate 80 surfactant is present in an amount of about 0.01% to about 0.1% (w/v), about 0.02% to about 0.09% (w/v), about 0.03% to about 0.08% (w/v), about 0.04% to about 0.07% (w/v), or about 0.05% to about 0.06% (w/v). In some aspects, the polysorbate 80 surfactant is present in an amount of at least about 0.01% (w/v), at least about 0.02% (w/v), at least about 0.03% (w/v), at least about 0.04% (w/v), at least about 0.05% (w/v), at least about 0.06% (w/v), at least about 0.07% (w/v), at least about 0.08% (w/v), at least about 0.09% (w/v) or at least about 0.1% (w/v). In some aspects, the surfactant mitigates particulate and/or air bubble formation when agitated on a shaker.

In some aspects, the pharmaceutical formulation further comprises an amino acid buffering agent. In some aspects, the amino acid buffering agent is histidine. In some aspects, the histidine buffering agent is present in an amount of about 10 mM to about 100 mM histidine, about 20 mM to about 90 mM histidine, about 30 mM to about 80 mM histidine, about 40 mM to about 70 mM histidine, about 10 mM to about 30 mM histidine, about 15 mM to about 25 mM histidine, about 17.5 to about 22.5 histidine, or about 40 mM to about 60 mM histidine. In some aspects, the histidine buffering agent is present in an amount of about 10 mM histidine, about 15 mM histidine, about 20 mM histidine, about 25 mM histidine, about 30 mM histidine, about 35 mM histidine, about 40 mM histidine, about 45 mM histidine or about 50 mM histidine.

In some aspects, the pharmaceutical formulation further comprises an osmotic regulator. In some aspects, the osmotic regulator comprises a sugar. In some aspects, the sugar is sucrose. In some aspects, the sucrose osmotic regulator is present in an amount of about 100 mM to about 1 M sucrose, about 200 mM to about 900 mM, about 300 mM to about 800 mM, about 400 mM to about 700 mM, or about 500 mM to about 600 mM. In some aspects, the sucrose osmotic regulator is present in an amount of about 100 mM sucrose, about 200 mM sucrose, about 300 mM sucrose, about 400 mM sucrose, about 500 mM sucrose, about 600 mM sucrose, about 700 mM sucrose, about 800 mM sucrose, about 900 mM sucrose, or about 1M sucrose.

In some aspects, the formulation is formulated for subcutaneous administration. In some aspects, the formulation is formulated for subcutaneous administration with a safety syringe. In some aspects, the formulation is formulated for daily or weekly administration. In some aspects, the formulation is an aqueous formulation.

The present disclosure provides a pharmaceutical formulation comprising:

(i) a FGF-21 conjugate;
(ii) histidine at a concentration between about 10 mM and about 50 mM;
(iii) sucrose at a concentration between about 100 mM and about 1M;
(iv) Polysorbate 80 at a concentration between about 0.01% and about 0.1% (w/v); and,
(v) DTPA at a concentration between about 10 μM and about 100 μM;
wherein the pH of the formulation is between about 6.7 and about 7.5,
wherein the FGF-21 conjugate comprises formula I:

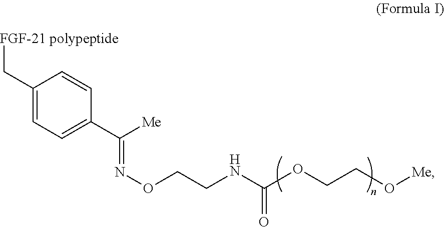

wherein n is between about 670 and about 690, e.g., about 681, and wherein the FGF-21 polypeptide comprises SEQ ID NO: 1.

The present disclosure also provides a pharmaceutical formulation comprising:

(i) a FGF-21 conjugate;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 μM;
wherein the pH is about 7.1,
wherein the FGF-21 conjugate comprises formula I:

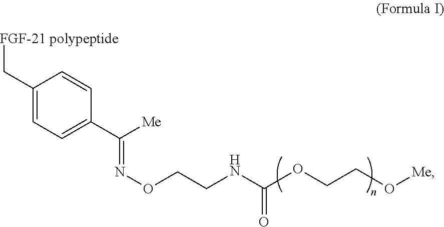

wherein n is between about 670 and about 690, e.g., about 681, and wherein the FGF-21 polypeptide comprises SEQ ID NO: 1.

Also provided is a pharmaceutical formulation comprising:

(i) a FGF-21 conjugate;
(ii) histidine at a concentration of 20 mM;
(iii) sucrose at a concentration of 600 mM;
(iv) Polysorbate 80 at a concentration of 0.05% (w/v); and
(v) DTPA at a concentration of 50 μM;
wherein the pH is 7.1,
wherein the FGF-21 conjugate comprises formula I:

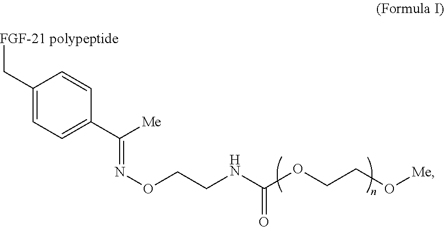

wherein n is between about 670 and about 690, e.g., about 681, and wherein the FGF-21 polypeptide comprises SEQ ID NO: 1.

The present disclosure provides a pharmaceutical formulation comprising:

(i) a FGF-21 conjugate;
(ii) histidine at a concentration of about 20 mM; and
(iii) sucrose at a concentration of about 600 mM;
wherein the pH is about 7.0,
wherein the FGF-21 conjugate comprises formula I:

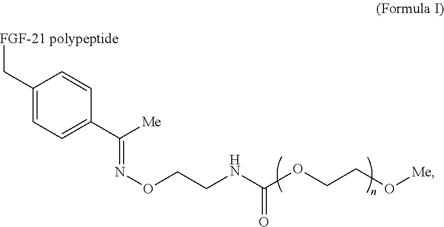

wherein n is between about 670 and about 690, e.g., about 681, and wherein the FGF-21 polypeptide comprises SEQ ID NO: 1.

The present disclosure provides a pharmaceutical formulation comprising:

(i) a FGF-21 conjugate;
(ii) histidine at a concentration of 20 mM; and
(iii) sucrose at a concentration of 600 mM;
wherein the pH is 7.0,
wherein the FGF-21 conjugate comprises formula I:

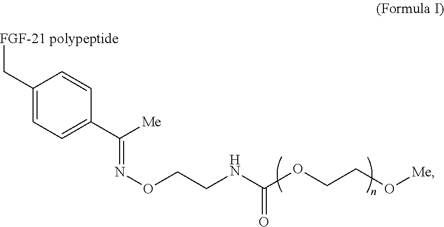

wherein n is between about 670 and about 690, e.g., about 681, and wherein the FGF-21 polypeptide comprises SEQ ID NO: 1.

The present disclosure also provides a pharmaceutical formulation comprising:

(i) FGF-21 conjugate at a concentration of about 10 mg/mL;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 uM;
wherein the pH is about 7.1,
wherein the FGF-21 conjugate comprises formula I:

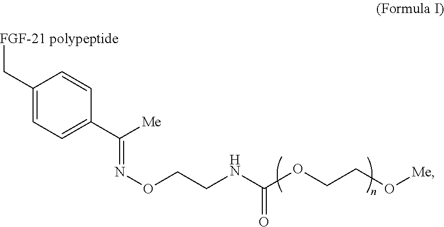

wherein n is between about 670 and about 690, e.g., about 681, and wherein the FGF-21 polypeptide comprises SEQ ID NO: 1.

The present disclosure also provides a pharmaceutical formulation comprising:

(i) FGF-21 conjugate at a concentration of about 20 mg/mL;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 uM;
wherein the pH is about 7.1,
wherein the FGF-21 conjugate comprises formula I:

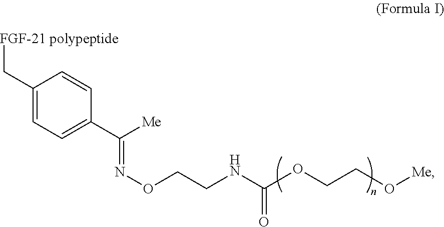

wherein n is between about 670 and about 690, e.g., about 681, and wherein the FGF-21 polypeptide comprises SEQ ID NO: 1.

The present disclosure provides a pharmaceutical formulation comprising:

(i) FGF-21 conjugate at a concentration of 10 mg/mL;
(ii) histidine at a concentration of 20 mM;
(iii) sucrose at a concentration of 600 mM;
(iv) Polysorbate 80 at a concentration of 0.05% (w/v); and
(v) DTPA at a concentration of 50 uM;
wherein the pH is 7.0,
wherein the FGF-21 conjugate comprises formula I:

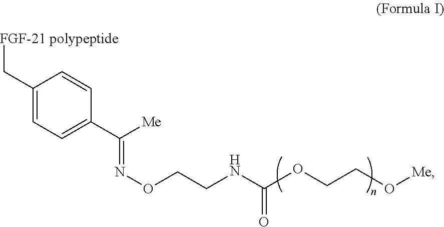

wherein n is between about 670 and about 690, e.g., about 681, and wherein the FGF-21 polypeptide comprises SEQ ID NO: 1.

The present disclosure provides a pharmaceutical formulation comprising:

(i) PEG-FGF21 of SEQ ID NO: 2 or 4 at a concentration of 20 mg/mL;
(ii) histidine at a concentration of 20 mM;
(iii) sucrose at a concentration of 600 mM;
(iv) Polysorbate 80 at a concentration of 0.05% (w/v); and
(v) DTPA at a concentration of 50 uM;
wherein the pH is 7.0,
wherein the FGF-21 conjugate comprises formula I:

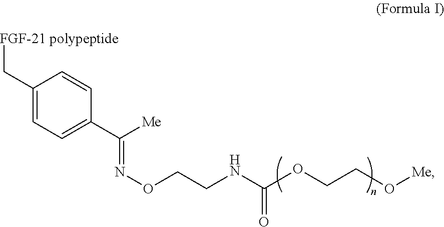

wherein n is between about 670 and about 690, e.g., about 681, and wherein the FGF-21 polypeptide comprises SEQ ID NO: 1.

The present disclosure provides a method to improve the stability of a pharmaceutical formulation comprising a fibroblast growth factor 21 (FGF-21) polypeptide conjugated to a polyethylene glycol (PEG) moiety (“FGF-21 conjugate”), the method comprising admixing an aminopolycarboxylic acid cation chelator, wherein the formulation has improved stability compared to a reference formulation that does not contain the aminopolycarboxylic acid cation chelator, wherein the FGF-21 conjugate comprises formula I:

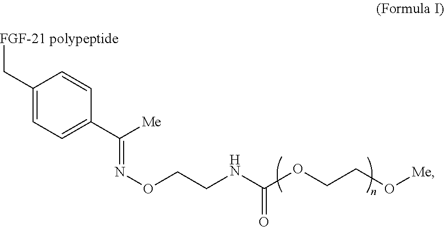

wherein n is between about 670 and about 690, e.g., about 681, and wherein the FGF-21 polypeptide comprises SEQ ID NO: 1.

In some aspects of the methods disclosed herein, the PEG moiety is conjugated to a non-natural amino acid in the FGF-21 polypeptide. In some aspects, the non-natural amino acid in the FGF-21 polypeptide is para-acetyl-L-phenylalanine. In some aspects, the FGF-21 polypeptide is a FGF-21 polypeptide of SEQ ID NO: 1. In some aspects, the FGF-21 conjugate is in an L conformation. In some aspects, the FGF-21 conjugate is in a D conformation. In some aspects, the improvement in stability comprises (i) an increase in polypeptide physical stability, (ii) an increase in polypeptide chemical stability, or (iii) both (i) and (ii). In some aspects, the increase in physical stability comprises (i) prevention or decrease of polypeptide aggregation, (ii) prevention or decrease of polypeptide fragmentation, or (iii) both (i) and (ii). In some aspects, the increase in chemical stability comprises (i) prevention or decrease of polypeptide deamidation, (ii) prevention or decrease of polypeptide oxidation, or (iii) both (i) and (ii). In some aspects, the improvement in stability comprises one or more of: (a) a lower rate of polypeptide deamidation when stored at 40° C. for about a month with respect to the reference formulation; (b) a lower rate of high molecular weight (HMW) polypeptide aggregation when stored at 40° C. for about a month with respect to the reference formulation; or (c) both (a) and (b). In some aspects, the improvement in stability comprises preventing or mitigating oxidation of one or more methionines. In some aspects, the methionines correspond to Met1 and/or Met169 of the FGF-21 polypeptide. In some aspects, methionine oxidation is prevented or mitigated at 25° C. and/or 40° C.

In some aspects of the methods disclosed herein, the aminopolycarboxylic acid cation chelator is DTPA. In some aspects, the DTPA cation chelator is present in an amount between about 10 μM and about 100 μM DTPA, between about 20 μM and about 90 μM DTPA, between about 25 μM and about 75 μM DTPA, between about 40 μM and about 60 μM DTPA, between about 30 μM and about 70 μM DTPA, between about 30 μM and about 80 μM DTPA, or between about 40 μM and about 70 μM. In some aspects, the DTPA cation chelator is present in an amount of about 40 μM, about 45 μM, about 50 μM, about 55 μM, or about 60 μM DTPA.

In some aspects of the methods disclosed herein, the method further comprises adjusting the pH to above 6.5, above 6.6, above 6.7, above 6.8, above 6.9, or above 7.0. In some aspects, the pH is adjusted to between about 6.8 and about 7.5, or between about 6.9 and about 7.4, or between about 7.0 and about 7.3, or between about 7.1 and 7.2, or between about 7.1 and about 7.3, or between about 7.1 and about 7.4, or between about 7.1 and about 7.5. In some aspects, the adjusted pH is about 6.8, about 6.9, about 7.0, about 7.1, about 7.2, about 7.3, about 7.4, or about 7.5. In some aspects, the formulation is more stable than the reference formulation having a pH of 6.5.

In some aspects of the methods disclosed herein, the method further comprises admixing a surfactant. In some aspects, the surfactant is a nonionic surfactant. In some aspects, the nonanionic surfactant is a polysorbate. In some aspects, the polysorbate is polysorbate 80. In some aspects, the polysorbate 80 surfactant is admixed in an amount of about 0.01% to about 0.1% (w/v), about 0.02% to about 0.09% (w/v), about 0.03% to about 0.08% (w/v), about 0.04% to about 0.07% (w/v), or about 0.05% to about 0.06% (w/v). In some aspects, polysorbate 80 surfactant is admixed in an amount of at least about 0.01% (w/v), at least about 0.02% (w/v), at least about 0.03% (w/v), at least about 0.04% (w/v), at least about 0.05% (w/v), at least about 0.06% (w/v), at least about 0.07% (w/v), at least about 0.08% (w/v), at least about 0.09% (w/v) or at least about 0.1% (w/v). In some aspects, the surfactant mitigates particulate formation and/or air bubble formation when agitated on a shaker.

In some aspects of the methods disclosed herein, the method further comprises admixing an amino acid buffering agent. In some aspects, the amino acid buffering agent is histidine. In some aspects, the histidine buffering agent is admixed in an amount of about 10 mM to about 100 mM histidine, about 20 mM to about 90 mM histidine, about 30 mM to about 80 mM histidine, about 40 mM to about 70 mM histidine, about 10 mM to about 30 mM histidine, about 15 mM to about 25 mM histidine, about 17.5 mM to about 22.5 mM histidine, or about 40 mM to about 60 mM histidine. In some aspects, the histidine buffering agent is admixed in an amount of about 10 mM histidine, about 15 mM histidine, about 20 mM histidine, about 25 mM histidine, about 30 mM histidine, about 35 mM histidine, about 40 mM histidine, about 45 mM histidine or about 50 mM histidine.

In some aspects of the methods disclosed herein, the method further comprises admixing an osmotic regulator. In some aspects, the osmotic regulator comprises a sugar. In some aspects, the sugar is sucrose. In some aspects, the sucrose osmotic regulator is admixed in an amount of about 100 mM to about 1 M sucrose, about 200 mM to about 900 mM sucrose, about 300 mM to about 800 mM sucrose, about 400 mM to about 700 mM sucrose, or about 500 mM to about 600 mM sucrose. In some aspects, the sucrose osmotic regulator is admixed in an amount of about 100 mM sucrose, about 200 mM sucrose, about 300 mM sucrose, about 400 mM sucrose, about 500 mM sucrose, about 600 mM sucrose, about 700 mM sucrose, about 800 mM sucrose, about 900 mM sucrose, or about 1M sucrose.

In some aspects, the formulation is an aqueous formulation. In some aspects, the present disclosure provides a pharmaceutical formulation prepared according to the methods disclosed herein.

The present disclosure also provides a vial comprising:

(i) a FGF-21 conjugate;
(ii) histidine at a concentration between about 10 mM and about 50 mM;
(iii) sucrose at a concentration between about 100 mM and about 1M;
(iv) Polysorbate 80 at a concentration between about 0.01% and about 0.1% (w/v); and,
(v) DTPA at a concentration between about 10 μM and about 100 μM;
wherein the pH of the formulation is between about 6.7 and about 7.5,
wherein the FGF-21 conjugate comprises formula I:

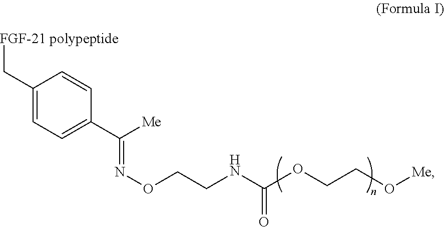

wherein n is between about 670 and about 690, e.g., about 681, and wherein the FGF-21 polypeptide comprises SEQ ID NO: 1.

The present disclosure also provides a vial comprising:

(i) about 5 mg to about 20 mg of a FGF-21 conjugate;
(ii) histidine at a concentration of about 20 mM; and
(iii) sucrose at a concentration of about 600 mM;
wherein the pH is about 7.0,
wherein the FGF-21 conjugate comprises formula I:

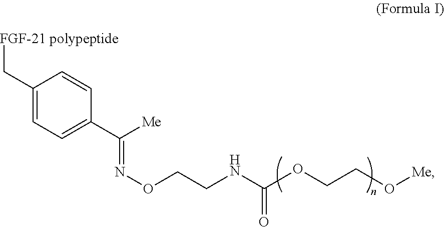

wherein n is between about 670 and about 690, e.g., about 681, and wherein the FGF-21 polypeptide comprises SEQ ID NO: 1.

The present disclosure also provides a vial comprising:

(i) about 10 mg to about 20 mg of a FGF-21 conjugate;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 uM;
wherein the pH is about 7.1,
wherein the FGF-21 conjugate comprises formula I:

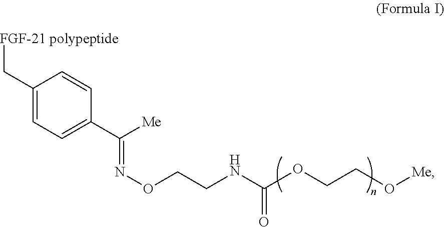

wherein n is between about 670 and about 690, e.g., about 681, and wherein the FGF-21 polypeptide comprises SEQ ID NO: 1.

The present disclosure also provides a vial comprising:

(i) about 10 mg of a FGF-21 conjugate;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 uM;
wherein the pH is about 7.1,
wherein the FGF-21 conjugate comprises formula I:

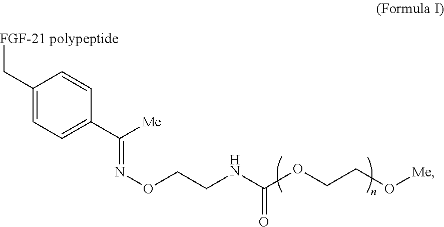

wherein n is between about 670 and about 690, e.g., about 681, and wherein the FGF-21 polypeptide comprises SEQ ID NO: 1.

The present disclosure also provides a vial comprising:

(i) about 20 mg of a FGF-21 conjugate;
(ii) histidine at a concentration of about 20 mM;
(iii) sucrose at a concentration of about 600 mM;
(iv) Polysorbate 80 at a concentration of about 0.05% (w/v); and
(v) DTPA at a concentration of about 50 uM;
wherein the pH is about 7.1,
wherein the FGF-21 conjugate comprises formula I:

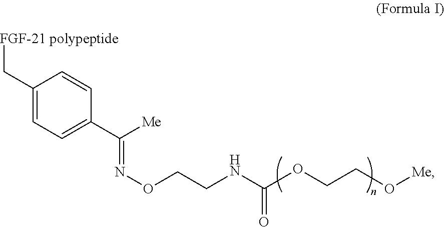

wherein n is between about 670 and about 690, e.g., about 681, and wherein the FGF-21 polypeptide comprises SEQ ID NO: 1.

The present disclosure also provides a kit or article of manufacture comprising (i) the pharmaceutical formulation disclosed herein or a vial disclosed herein, and (ii) instructions for use.

The present disclosure also provides method of treating or preventing a disease or condition associated with fibrosis and/or diabetes in a subject in need thereof comprising administering to the subject an effective amount of a pharmaceutical formulation disclosed herein, a vial disclosed herein, or a kit disclosed herein. In some aspects, the disease or condition is diabetes. In some aspects, the diabetes is type 2 diabetes. In some aspects, the disease or condition is nonalcoholic steatohepatitis (NASH). In some aspects, administration of the effective amount of the pharmaceutical formulation to the subject decreases liver stiffness, decreases percentage body fat, decreases body weight, decreases liver-to-body weight ratio, decreases liver lipid content, decreases liver fibrosis area, decreases fasting blood glucose levels, decreases fasting triglyceride levels, decreases LDL cholesterol levels, decreases ApoB levels, decreases ApoC levels, increases HDL cholesterol, or any combination thereof. In some aspects, the FGF-21 conjugate is administered at a flat dose of about 20 mg. In some aspects, the FGF-21 conjugate is administered at a dosing interval of a week. In some aspects, the pharmaceutical formulation is administered subcutaneously. In some aspects, the pharmaceutical formulation is administered subcutaneously using a safety syringe. In some aspects, the administration of the pharmaceutical formulation to the subject results in

(i) reduction in levels of liver fat;
(ii) reduction in levels of liver injury;
(iii) reduction in levels of fibrosis;
(iv) decrease in levels of fibrosis biomarker serum Pro-C3 (N-terminal type III collagen propeptide);
(v) decrease in levels of alanine aminotransferase (ALT);
(vi) decrease in levels of aspartate aminotransferase (AST),
(vii) increase in levels of serum adiponectin;
(viii) decrease in levels of plasma LDL
(ix) increase in levels of plasma HDL;
(x) decrease in levels of plasma triglyceride;
(xi) reduction in level of liver stiffness; or
(xii) any combination thereof,
compared to the levels in untreated subjects or to the subject prior to the administration of the pharmaceutical formulation.

In some aspects, FGF-21 polypeptide can be conjugated to a PEG that is about 30 kDa.

### BRIEF DESCRIPTION OF THE DRAWINGS

FIGS. 1A and 1B show deamidation of PEG-FGF-21 as a function of formulation composition. FIG. 1A shows deamidation in a formulation without pentetic acid (DTPA) or polysorbate 80. FIG. 1B shows deamidation in a formulation comprising pentetic acid (DTPA) and polysorbate 80.

FIGS. 2A and 2B show metal catalyzed oxidation of PEG-FGF-21 polypeptide at methionine 1 (FIG. 2A) and methionine 169 (FIG. 2B) in the presence and absence of 50 μM DTPA (also known as pentetic acid).

FIGS. 3A and 3B show the effect of pH on PEG-FGF-21 aggregation. FIG. 3A shows aggregation as a function of time, pH, and buffer system used. FIG. 3B shows aggregation at different temperatures and times (5° C., 14 months; 25° C., 1 month; 40° C., 1 day) and different pH.

FIGS. 4A and 4B show aggregation of PEG-FGF-21 as a function of formulation composition. FIG. 4A shows aggregation in a formulation without pentetic acid (DTPA) or polysorbate 80. FIG. 4B shows aggregation in a formulation with pentetic acid (DTPA) and polysorbate 80.

FIG. 5 shows that PEG-FGF-21 at higher concentration without PS80 turned cloudy after 300 rpm orbital shaker 24 h (left) or wristaction shaking for 6 h (right). Addition of polysorbate 80 between 0.01% and 0.1% (w/v) reduced the cloudiness of the samples.

FIG. 6 shows a schematic representation of a PEGylated FGF-21 conjugate of the present disclosure; in particular, an example in which the non-natural amino acid in the FGF-21 conjugate is para-acetyl-phenylalanine, e.g., para-acetyl-L-phenylalanine. n represents the number of ethylene glycol units contained in the PEG polymer.

FIG. 7 shows a schematic representation of a specific PEG linker comprising 681 ethylene glycol units that can be fused or conjugated in a site specific manner to a FGF-21 polypeptide (e.g., a FGF-21 polypeptide comprising a non-native amino acid such as para-acetyl-phenylalanine) to yield a FGF-21 conjugate of the present disclosure.

### DETAILED DESCRIPTION

The present disclosure provides a stabilized pharmaceutical formulation comprising a fibroblast grow factor 21 (FGF-21) conjugate, e.g., such as PEG-FGF-21 (e.g., SEQ ID NO: 2 or 4). The presence of an aminopolycarboxylic acid cation chelator such as DTPA has been observed to mitigate oxidation of one or more amino acid residues, for example, methionines, in a FGF-21 conjugate disclosed herein, e.g., a PEG-FGF-21 of SEQ ID NO: 2 or SEQ ID NO:4.

Incorporation of DTPA in a formulation comprising a FGF-21 conjugate disclosed herein, e.g., a PEG-FGF-21 of SEQ ID NO: 2 or SEQ ID NO: 4, also lowers the rate of deamidation of the FGF-21 polypeptide during storage, and also reduces the rate of high molecular weight aggregation during storage. Further stabilization can be achieved by adjusting the pH of the formulation to 7.1. Additionally, the formulation can be further stabilized by adding a surfactant such as polysorbate 80.

The disclosure also provides methods to manufacture the disclosed formulation, as well as formulations produced by applying the disclosed method. Also provided are methods of treatment or prophylaxis of diseases associated with fibrosis, e.g., NASH and diabetes, comprising the administration of the disclosed stabilized formulations to a subject in need thereof.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The disclosure includes aspects in which exactly one member of the group is present in, employed in, or otherwise relevant to a given product or process. The disclosure includes aspects in which more than one, or all of the group members are present in, employed in, or otherwise relevant to a given product or process.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.” Thus, for example, reference to a “FGF-21 conjugate” is a reference to one or more such proteins or conjugates and includes equivalents thereof known to those of ordinary skill in the art, and so forth.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

Aggregation: The term “aggregation” refers to the tendency of a polypeptide, e.g., a FGF-21 polypeptide moiety of a FGF-21 conjugate disclosed herein, e.g., a PEG-FGF-21 of SEQ ID NO: 2 or SEQ ID NO:4, form non-covalently linked complexes with other molecules (such as other molecules of the same polypeptide) thereby forming high molecular weight complexes. Exemplary methods of measuring the formation of aggregates include analytical size exclusion chromatography as described in the Examples herein. Relative amounts of aggregation may be determined with respect to a reference compound, e.g., to identify a polypeptide having reduced aggregation. Relative amounts of aggregation can also be determined with respect to a reference formulation, e.g., to identify a formulation in which, for example, a FGF-21 conjugate disclosed herein, e.g., a PEG-FGF-21 of SEQ ID NO: 2 or SEQ ID NO:4, has reduced aggregation.

Amino acid substitution: The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a disease, the term “associated with” means that the symptom, measurement, characteristic, or status in question is linked to the diagnosis, development, presence, or progression of that disease. As association may, but need not, be causatively linked to the disease.

Biologically active: The term “biologically active” as applied to a molecule disclosed herein, for example, a FGF-21 conjugate disclosed herein, e.g., a PEG-FGF-21 of SEQ ID NO: 2 or SEQ ID NO:4, means any substance which can affect any physical or biochemical properties of a biological system, pathway, molecule, or interaction relating to a living organism. In particular, as used herein, biologically active molecules include, but are not limited to, any substance intended for diagnosis, cure, mitigation, treatment, or prevention of disease or conditions, e.g., diseases or conditions associated with fibrosis, in humans or other animals, or to otherwise enhance physical or mental well-being of humans or animals.

Conservative amino acid substitution: A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Conserved: As used herein, the term “conserved” refers to amino acid residues of a polypeptide sequence, respectively, that are those that occur unaltered in the same position of two or more sequences being compared. Amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Deamidation: The term “deamidation” refers to the tendency of amino acid residues within a polypeptide to spontaneously undergo a deamidation reaction, thereby changing the chemical structure of the amino acid, and potentially affecting the function of the polypeptide. Exemplary methods of measuring deamidation are disclosed in the Examples herein. The relative amount of deamidation may be determined with respect to a reference compound, e.g., to identify a polypeptide having decreased deamidation. Relative amounts of deamidation can also be determined with respect to a reference formulation, e.g., to identify a formulation in which a FGF-21 conjugate disclosed herein, e.g., a PEG-FGF-21 of SEQ ID NO: 2 or SEQ ID NO:4, has reduced deamidation.

Disease associated with fibrosis: The term “disease associated with fibrosis” includes diseases, disorders, and conditions in which fibrosis has been observed to occur or in which fibrosis is known or thought to be associated with or contribute to disease etiology, progression, or symptoms, or in which fibrosis is known or thought to occur as the disease progresses.

The fibrosis may affect an organ or tissue such as the pancreas, lung, heart, kidney, liver, eyes, nervous system, bone marrow, lymph nodes, endomyocardium, or retroperitoneum. Exemplary diseases associated with fibrosis include, but are not limited to nonalcoholic steatohepatitis (NASH), liver fibrosis, pre-cirrhosis, cirrhosis, diffuse parenchymal lung disease, cystic fibrosis, lung or pulmonary fibrosis, progressive massive fibrosis, idiopathic pulmonary fibrosis, injection fibrosis, kidney or renal fibrosis, chronic kidney disease, diabetic kidney disease, focal segmental glomerulosclerosis, membranous nephropathy, IgA nephropathy, myelofibrosis, heart failure, metabolic heart failure, cardiac fibrosis, cataract fibrosis, cataract, ocular scarring, pancreatic fibrosis, skin fibrosis, intestinal fibrosis, intestinal strictures, endomyocardial fibrosis, atrial fibrosis, mediastinal fibrosis, Crohn's disease, retroperitoneal fibrosis, keloid, nephrogenic systemic fibrosis, scleroderma, systemic sclerosis, arthrofibrosis, Peyronie's syndrome, Dupuytren's contracture, diabetic neuropathy, adhesive capsulitis, alcoholic liver disease, hepatosteatosis, viral hepatitis, biliary disease, primary hemochromatosis, drug-related cirrhosis, cryptogenic cirrhosis, Wilson's disease, and, alpha 1-antitrypsin deficiency, interstitial lung disease (ILD), human fibrotic lung disease, macular degeneration, retinal retinopathy, vitreal retinopathy, myocardial fibrosis, Grave's ophthalmopathy, drug induced ergotism, cardiovascular disease, atherosclerosis/restenosis, hypertrophic scars, primary or idiopathic myelofibrosis, and inflammatory bowel disease (including, but not limited to, collagenous colitis). In some aspects, the disease associated with fibrosis can include liver fibrosis, kidney or renal fibrosis, lung or pulmonary fibrosis and heart or cardiac fibrosis. In some aspects, the disease associated with fibrosis can be liver fibrosis. In some aspects, the disease associated with fibrosis can be NASH.

Effective Amount: As used herein, the term “effective amount” of a formulation comprising a FGF-21 conjugate disclosed herein, e.g., a PEG-FGF-21 of SEQ ID NO: 2 or SEQ ID NO:4, is that amount sufficient to effect beneficial or desired results, for example, clinical results, and, as such, an “effective amount” depends upon the context in which it is being applied. For example, in the context of administering a FGF-21 conjugate disclosed herein, e.g., a PEG-FGF-21 of SEQ ID NO: 2 or SEQ ID NO:4, that treats NASH, an effective amount of the FGF-21 conjugate is, for example, an amount sufficient to improve liver fat, liver injury or fibrosis (e.g., a reduction in liver fat, liver injury or fibrosis with respect to levels in untreated subjects or with respect to levels in the subject prior to the administration of the treatment). In some aspects, the amount of the FGF-21 polypeptide in the formulation disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

In some aspects, an effective amount of a FGF-21 conjugate disclosed herein, e.g., a PEG-FGF-21 of SEQ ID NO: 2 or SEQ ID NO:4, to treat NASH can change the level of one or more fibrosis biomarkers: for example, decrease serum Pro-C3; decrease ALT or AST; increase serum adiponectin; decrease plasma LDL; increase plasma HDL; decrease plasma triglyceride levels, or any combination thereof, with respect to levels in untreated subjects or with respect to levels in the subject prior to the administration of the treatment.

The term “effective amount” can be used interchangeably with “effective dose,” “therapeutically effective amount,” or “therapeutically effective dose.”

The “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the molecule, e.g., mg. As opposed to flat dose, the term “weight-based dose” as referred to herein means that a dose that is administered to a patient is calculated based on the weight of the patient. In some aspects, the amount of the FGF-21 polypeptide in the flat dose disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

FGF-21 activity: The term “FGF-21 activity” refers to at least one biological activity of a FGF-21 polypeptide in the FGF-21 conjugate (e.g., a PEGylated FGF-21 conjugate of the present disclosure). The term “biological activity” refers to the ability of a molecule, e.g., an FGF-21 polypeptide (e.g., a FGF-21 polypeptide in a FGF-21 conjugate or a FGF-21 conjugate disclosed herein, e.g., a PEG-FGF-21 of SEQ ID NO: 2 or SEQ ID NO:4) to affect any physical or biochemical properties of a biological system, pathway, molecule, or interaction relating to an organism, including but not limited to, viruses, bacteria, bacteriophage, transposon, prion, insects, fungi, plants, animals, and humans.

For example, biological activity includes any of the biological functions performed by wild-type FGF-21. Exemplary methods of determining whether a molecule possesses at least one biological activity of wild-type FGF-21 (such as the wild-type FGF-21 polypeptide of SEQ ID NO: 3) can include any functional assays known in the art, including the methods disclosed in Example 5 and 17 of U.S. Appl Publ. No. 2017/0189486, which is herein incorporated by reference in its entirety.

Reference compound: The relative level of biological activity can be determined with respect to a reference compound, e.g., to identify a polypeptide having biological activity or having sufficiently high biological activity for an intended therapeutic use, e.g., having an EC₅₀ less than 5-fold, less than 10-fold, less than 20-fold, less than 50-fold, or less than 100-fold higher than the EC₅₀ of a reference compound. The relative level of biological activity can also be determined with respect to a reference formulation, e.g., to identify a formulation in which a FGF-21 conjugate disclosed herein, e.g., a PEG-FGF-21 of SEQ ID NO: 2 or SEQ ID NO:4, has biological activity or has sufficiently high biological activity for an intended therapeutic use, e.g., having an EC₅₀ less than 5-fold, less than 10-fold, less than 20-fold, less than 50-fold, or less than 100-fold higher than the EC₅₀ of corresponding FGF-21 in the reference formulation.

The reference compound described herein can be a corresponding FGF-21 sequence lacking a modification, such as PEGylation as described herein. For example, the reference compound can be wild type FGF-21, or a variant FGF-21 polypeptide sequence (e.g., a wild type FGF-21 with an amino acid substitution, such as the replacement of Q109 with a non-natural amino acid such as para-acetyl-L-phenylalanine) but without a PEG conjugate moiety. Thus, in some aspects, a reference compound can contain at least one non-natural amino acid, which is not linked to a PEG moiety described herein. In some aspects, the PEG moiety comprises a distal methoxy (—O—CH₃) group.

Exemplary reference compounds for FGF-21 conjugates disclosed herein, e.g., a PEG-FGF-21 of SEQ ID NO: 2 or SEQ ID NO:4, include without limitation the wild-type FGF-21 of SEQ ID NO: 3, and the variant FGF-21 polypeptide of SEQ ID NO: 1.

In some aspects, a reference compound may contain additional amino acid substitutions, deletions, and/or insertions. In some aspects, the comparison can be performed with a PEGylated or non-PEGylated form of the polypeptide; in the former instance, the comparison can be performed with a polypeptide comprising or not comprising a non-natural amino acid.

Identity: As used herein, the term “identity” refers to the overall monomer conservation between polymeric molecules, e.g., between polypeptide molecules. The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. Sequence alignments can be conducted using methods known in the art such as MAFFT, Clustal (ClustalW, Clustal X or Clustal Omega), MUSCLE, etc.

Different regions within a single polypeptide target sequence that aligns with a polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence to a second amino acid sequence is calculated as % ID=100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

In vivo proteolytic degradation: The term “in vivo proteolytic degradation” refers to the cleavage of a polypeptide when introduced into a living system (e.g., when injected into an organism) which may result from proteases occurring in said organism. Proteolysis can potentially affect the biological activity or half-life of a polypeptide. For example, wild-type FGF-21 can undergo cleavage at the C-terminus, resulting in a truncated, inactive polypeptide.

An exemplary method of measuring in vivo proteolysis of FGF-21 is the Meso Scale Discovery (MSD)-based electrochemiluminescent immunosorbent assay (ECLIA) described in Example 10 of U.S. Appl. Publ. No. US2017/0189486. The relative amount of in vivo or in vitro proteolysis may be determined with respect to a reference compound, e.g., to identify a polypeptide having decreased in vivo proteolysis. The relative amount of in vivo proteolysis can also be determined with respect to a reference formulation, i.e., to identify a formulation in which the FGF-21 polypeptide moiety of a FGF-21 conjugate disclosed herein, e.g., a PEG-FGF-21 of SEQ ID NO: 2 or SEQ ID NO:4, has decreased in vitro proteolysis.

Isolated: As used herein, the term “isolated” refers to a substance or entity (e.g., a polypeptide) that has been separated from at least some of the components with which it was associated (whether in nature or in an experimental setting). Isolated substances (e.g., proteins) can have varying levels of purity in reference to the substances from which they have been associated.

Linked: The terms “linked,” “fused,” “conjugated” and “attached” are used interchangeably and refer to a PEG moiety (e.g., a ˜30 kD PEG moiety) covalently fused or concatenated, including internally inserted, to an FGF-21 polypeptide, e.g., a variant FGF-21 polypeptide disclosed herein (e.g., a FGF-21 polypeptide of SEQ ID NO:1).

In the context of the present disclosure, a FGF-21 polypeptide, e.g., a variant FGF-21 polypeptide disclosed herein (e.g., a FGF-21 polypeptide of SEQ ID NO:1), and a PEG moiety can be “fused” as a result of chemical synthesis.

In the context of the present disclosure, the terms “conjugate” or “conjugation” denote that two molecular entities (e.g., a FGF-21 polypeptide, e.g., a variant FGF-21 polypeptide disclosed herein and a polymer moiety such as PEG) have been chemically linked. In some particular aspects, the FGF-21 polypeptide and the PEG moiety are linked via an oxime linkage as shown in formula I disclosed herein. In some aspects, the PEG moiety comprises a distal methoxy (—O—CH₃) group.

Mutation: In the content of the present disclosure, the terms “mutation” and “amino acid substitution” as defined above (sometimes referred simply as a “substitution”) are considered interchangeable.

Non-natural amino acid: A “non-natural amino acid” refers to an amino acid that is not one of the 20 common amino acids or pyrrolysine or selenocysteine. Other terms that can be used synonymously with the term “non-natural amino acid” are “non-naturally encoded amino acid,” “unnatural amino acid,” “non-naturally occurring amino acid,” and various hyphenated and non-hyphenated versions thereof.

The term “non-natural amino acid” also includes, but is not limited to, amino acids that occur by modification (e.g., post-translational modifications) of a naturally encoded amino acid (including but not limited to, the 20 common amino acids) but are not themselves naturally incorporated into a growing polypeptide chain by the translation complex. Examples of such non-natural amino acids include, but are not limited to, N-acetylglucosaminyl-L-serine, N-acetylglucosaminyl-L-threonine, and O-phosphotyrosine. In one specific aspect of the present disclosure, a non-natural amino acid is para-acetyl-phenylalanine. In one specific aspect of the present disclosure, a non-natural amino acid is para-acetyl-L-phenylalanine. In one specific aspect of the present disclosure, a non-natural amino acid is para-acetyl-D-phenylalanine.

Patient: As used herein, “patient” refers to a subject who can seek or be in need of treatment, requires treatment, is receiving treatment, will receive treatment, or a subject who is under care by a trained professional for a particular disease or condition.

Pharmaceutical composition: The term “pharmaceutical composition” refers to a preparation which is in such form as to permit the biological activity of the active ingredient (e.g., a FGF-21 conjugate disclosed herein, e.g., a PEG-FGF-21 of SEQ ID NO: 2 or SEQ ID NO:4) to be effective, and which contains no additional components (e.g., excipients and water) which are unacceptably toxic to a subject to which the composition would be administered. Such composition can be sterile.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. In general, approval by a regulatory agency of the Federal or state governments (or listed in the U.S. Pharmacopeia or other generally recognized pharmacopeia) for use in animals, and more particularly in humans implies that those compounds, materials, compositions, and/or dosage forms are pharmaceutically acceptable. Compounds, materials, compositions, and/or dosage forms that are generally acceptable as safe for therapeutically purposes are “therapeutically acceptable.”

Pharmaceutically acceptable excipients: The phrase “pharmaceutically acceptable excipient,” as used herein, refers any ingredient other than the active compounds described herein (for example, a vehicle capable of suspending or dissolving the active compound) and having the properties of being substantially nontoxic and non-inflammatory in a subject. Excipients can include, for example chelators, surfactants, buffering agents, osmotic regulators, antioxidants, emulsifiers, fillers (diluents), preservatives, sorbents, suspensing or dispersing agents, sweeteners, and waters of hydration. Excipients that are generally accepted as safe for therapeutic purposes are “therapeutically acceptable excipients.”

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In a particular aspect, a polypeptide disclosed herein is a FGF-21 polypeptide.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, the pharmaceutical formulation disclosed in the present application can be used to prevent the onset, prevent the symptoms, or prevent complications of diseases or conditions associated with fibrosis such as NASH or diabetes.

Prophylactic: As used herein, “prophylactic” refers to a therapeutic or course of action used to prevent the onset of a disease or condition, or to prevent or delay a symptom of a disease or condition associated with fibrosis, e.g., NASH. In some aspect, the pharmaceutical formulations disclosed in the present application can be used prophylactically.

Prophylaxis: As used herein, a “prophylaxis” refers to a measure taken to maintain health and prevent or delay the onset of a disease or condition associates with fibrosis, e.g., NASH or diabetes, or to prevent or delay symptoms associated with a disease or condition.

Recombinant: A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The variant FGF-21s disclosed herein can be recombinantly produced using methods known in the art. The proteins and peptides disclosed herein can also be chemically synthesized.

In some aspects, a FGF-21 polypeptide useful for the conjugate disclosed herein (e.g., a FGF-21 polypeptide comprising a non-natural amino acid, e.g., an FGF-21 of SEQ ID NO:1) is recombinantly produced by a bacterial host.

Similarity: As used herein, the term “similarity” refers to the overall relatedness between polymeric molecules, e.g. between polypeptide molecules. Calculation of percent similarity of polymeric molecules to one another can be performed in the same manner as a calculation of percent identity, except that calculation of percent similarity takes into account conservative substitutions as is understood in the art.

Solubility: The term “solubility” refers to the amount of a substance that can dissolve in another substance, e.g., the amount of a wild type FGF-21 (e.g., a FGF-21 polypeptide of SEQ ID NO:3), variant FGF-21 (e.g., a FGF-21 polypeptide comprising a non-natural amino acid, e.g., a FGF-21 polypeptide of SEQ ID NO:1), or a FGF-21 conjugate of the present disclosure (e.g., a FGF-21 polypeptide of SEQ ID NO:2 or SEQ ID NO: 4) that can dissolve in an aqueous solution.

An exemplary method of measuring the solubility of a wild type, variant FGF-21 polypeptide, or a FGF-21 conjugate is the plug flow solubility test described in Example 8 of U.S. Appl. Publ. No. US2017/0189486. Relative solubility can be determined with respect to a reference compound, e.g., to identify a polypeptide having increased solubility. In some aspects, relative solubility can be determined with respect to a reference formulation, e.g., to identify a formulation in which the polypeptide has increase solubility.

Subject: By “subject” or “individual” or “animal” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain aspects, the mammal is a human subject. In other aspects, a subject is a human patient. In a particular aspect, a subject is a human patient or cells thereof whether in vivo, in vitro or ex vivo, amenable to the methods described herein.

Suffering from: An individual who is “suffering from” a disease, disorder, and/or condition has been diagnosed with or displays one or more symptoms of the disease, disorder, and/or condition. In some aspects, the pharmaceutical formulations disclosed herein can administered to a subject suffering from a disease or condition associated with fibrosis such as NASH or diabetes.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some aspects, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition.

In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition. In some aspects, the pharmaceutical formulations disclosed herein can be administered to a subject susceptible to a disease or condition associated with fibrosis such as NASH or diabetes.

Therapeutic Agent: The terms “therapeutic agent” or “agent” refers to a molecular entity that, when administered to a subject, has a therapeutic, diagnostic, and/or prophylactic effect and/or elicits a desired biological and/or pharmacological effect. For example, in some aspects, a FGF-21 conjugate disclosed herein (e.g., a PEG-FGF-21 of SEQ ID NO:2 or SEQ ID NO:4) can be a therapeutic agent. In some aspects, an agent is another molecule which is co-administered as part of a combination therapy with at least one FGF-21 conjugate disclosed herein, e.g., a PEG-FGF-21 of SEQ ID NO: 2 or SEQ ID NO:4.

Therapeutically effective amount: As used herein, the term “therapeutically effective amount” means an amount of an agent to be delivered (e.g., a FGF-21 conjugate disclosed herein, e.g., a PEG-FGF-21 of SEQ ID NO: 2 or SEQ ID NO:4, or a formulation comprising the FGF-21 conjugate) that is sufficient, when administered to a subject suffering from or susceptible to an infection, disease, disorder, and/or condition, to treat, improve symptoms of, diagnose, prevent, and/or delay the onset of the infection, disease, disorder, and/or condition. Generally, the administration of the therapeutically effective amount is expected to result in a therapeutically effective outcome.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treat, treatment, therapy: As used herein, the terms “treat” or “treatment” or “therapy” or grammatical variants thereof refer to partially or completely, preventing, alleviating, ameliorating, improving, relieving, delaying onset of, inhibiting progression of, reducing severity of, and/or reducing incidence of one or more symptoms or features of a disease or condition associated with fibrosis, e.g., NASH or diabetes. For example, “treating” a disease associated with fibrosis can refer to preventing symptoms, ameliorating symptoms, delaying the onset of the disease or condition or its symptoms, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

ug, uM, uL: As used herein, the terms “ug,” “uM,” and “uL” are used interchangeably with “μg,” “μM,” and “μL” respectively.

Various aspects of the disclosure are described in further detail in the following subsections.

### I. FGF-21 Conjugate Formulations

The present disclosure provides pharmaceutical formulations comprising a fibroblast growth factor 21 (FGF-21) conjugate and an aminopolycarboxylic acid cation chelator, e.g., diethylenetriaminepentaacetic acid (DTPA), wherein the formulations have improved stability compared to a reference formulation. The term “reference formulation” refers to a formulation comprising the same components and properties (e.g., pH, temperature) as a “test formulation” (formulation being compared to the reference formulation) except that it does not contain the aminopolycarboxylic acid cation chelator.

The term “FGF-21 conjugate” refers to a conjugate comprising a FGF-21 polypeptide moiety linked to a PEG moiety. In some aspects, the PEG moiety comprises a distal methoxy (—O—CH₃) group. The term “FGF-21 polypeptide” refers generically to both the wild-type FGF-21 polypeptide (e.g., a polypeptide of SEQ ID NO:3) and to a “variant FGF-21 polypeptide.”

As used herein, the terms “PEG-FGF-21 conjugate” or “PEG-FGF-21” refer to PEGylated FGF-21 forms comprising a PEG moiety linked to a variant FGF-21 polypeptide moiety via an oxime linkage. Exemplary PEG-FGF-21 conjugates of the present disclosure are set forth in SEQ ID NO:2 or 5 and SEQ ID NO:4 or 6 in TABLE 4, below.

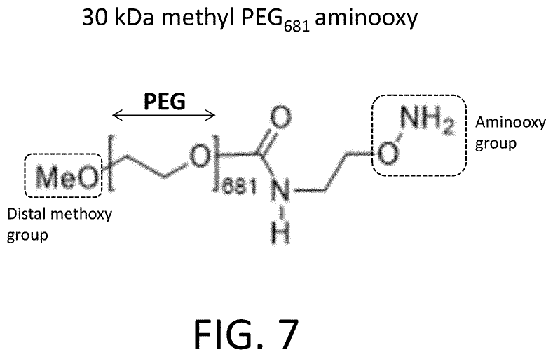

TABLE 1
Exemplary FGF-21 sequences

| SEQ ID | | |
|---|---|---|
| NO | Description | Sequence |
| 1 | Q109pAF FGF-21 | MHPIPDSSPLLQFGGQVRQRYLYTDDAQQTEAHLEIREDGTVGGAADQS |
| | | PESLLQLKALKPGVIQILGVKTSRFLCQRPDGALYGSLHFDPEACSFRE |
| | | LLLEDGYNVY(pAF)SEAHGLPLHLPGNKSPHRDPAPRGPARFLPLPGL |
| | | PPAPPEPPGILAPQPPDVGSSDPLSMVGPSQGRSPSYAS |
| | | pAF = para-acetyl-L-phenylalanine |
| 2 | PEG-FGF-21 | MHPIPDSSPLLQFGGQVRQRYLYTDDAQQTEAHLEIREDGTVGGAADQS |
| | Q109pAF FGF-21 | PESLLQLKALKPGVIQILGVKTSRFLCQRPDGALYGSLHFDPEACSFRE |
| | PEGylated | LLLEDGYNVY(pAF)SEAHGLPLHLPGNKSPHRDPAPRGPARFLPLPGL |
| | | PPAPPEPPGILAPQPPDVGSSDPLSMVGPSQGRSPSYAS |
| | | wherein pAF is linked to a 28-32 kDa PEG |
| 5 | PEG-FGF-21 without Met | HPIPDSSPLLQFGGQVRQRYLYTDDAQQTEAHLEIREDGTVGGAADQSP |
| | Q109pAF FGF-21 | ESLLQLKALKPGVIQILGVKTSRFLCQRPDGALYGSLHFDPEACSFREL |
| | PEGylated | LLEDGYNVY(pAF)SEAHGLPLHLPGNKSPHRDPAPRGPARFLPLPGLP |
| | | PAPPEPPGILAPQPPDVGSSDPLSMVGPSQGRSPSYAS |
| | | wherein pAF is linked to a 28-32 kDa PEG |
| 3 | Native FGF-21 | MHPIPDSSPLLQFGGQVRQRYLYTDDAQQTEAHLEIREDGTVGGAADQS |
| | | PESLLQLKALKPGVIQILGVKTSRFLCQRPDGALYGSLHFDPEACSFRE |
| | | LLLEDGYNVYQSEAHGLPLHLPGNKSPHRDPAPRGPARFLPLPGLPPAP |
| | | PEPPGILAPQPPDVGSSDPLSMVGPSQGRSPSYAS |
| | | The glutamine (Q) modified in SEQ ID NOS: 1, 2 and |
| | | 4 is underlined. |
| 4 | PEG-FGF-21 | MHPIPDSSPLLQFGGQVRQRYLYTDDAQQTEAHLEIREDGTVGGAADQS |
| | Q109pAF FGF-21 | PESLLQLKALKPGVIQILGVKTSRFLCQRPDGALYGSLHFDPEACSFRE |
| | PEGylated | LLLEDGYNVY(pAF)SEAHGLPLHLPGNKSPHRDPAPRGPARFLPLPGL |
| | PEG = PEG681 | PPAPPEPPGILAPQPPDVGSSDPLSMVGPSQGRSPSYAS |
| | | wherein pAF is linked to PEG₆₈₁ (linear PEG |
| | | comprising 681 ethylene glycol units) via an oxime |
| | | linkage, e.g., formed by reaction between the |
| | | acetyl group of pAF and the aminooxy group of the |
| | | 30 kDa methyl PEG₆₈₁ aminooxy molecule presented |
| | | in FIG. 7. |
| 6 | PEG-FGF-21 without Met | HPIPDSSPLLQFGGQVRQRYLYTDDAQQTEAHLEIREDGTVGGAADQSP |
| | Q109pAF FGF-21 | ESLLQLKALKPGVIQILGVKTSRFLCQRPDGALYGSLHFDPEACSFREL |
| | PEGylated | LLEDGYNVY(pAF)SEAHGLPLHLPGNKSPHRDPAPRGPARFLPLPGLP |
| | PEG = PEG681 | PAPPEPPGILAPQPPDVGSSDPLSMVGPSQGRSPSYAS |
| | | wherein pAF is linked to PEG₆₈₁ (linear PEG |
| | | comprising 681 ethylene glycol units) via an oxime |
| | | linkage, e.g., formed by reaction between the |
| | | acetyl group of pAF and the aminooxy group of the |
| | | 30 kDa methyl PEG₆₈₁ aminooxy molecule presented |
| | | in FIG. 7. |

Numerous FGF-21s known in the art can be used in the PEGylated FGF-21 conjugate formulations disclosed herein, for example those disclosed in U.S. Pat. Nos. 8,012,931 and 9,434,788, both of which are herein incorporated by reference in their entireties. Fibroblast growth factor 21 (FGF-21) has been described in the literature (Nishimura et al., Biochimica et Biophysica Acta, 1492:203-206 (2000); WO 01/36640; and WO 01/18172, and U.S. Patent Publication No. 20040259780, each of which is incorporated by reference herein in its entirety). Unlike other FGFs, FGF-21 has been reported not to have proliferative and tumorigenic effects (Ornitz and Itoh, Genome Biology 2001, 2(3):reviews3005.1-3005.12).

In some aspects, FGF-21 polypeptides useful for the present disclosure comprise the amino acid sequence as set forth in SEQ ID NO: 2 or SEQ ID NO: 4 without Methione at the N terminus.

Multiple polymorphisms of FGF-21 have been identified. Leucine or proline have been described at the same position in U.S. Patent Publication No. 20010012628 and U.S. Pat. No. 6,716,626. N-terminal leader or signal sequences that differ by one amino acid (leucine) are shown in U.S. Pat. No. 6,716,626 and U.S. Patent Publication No. 20040259780. FGF-21 variants or mutants include, but are not limited to, those disclosed in U.S. Pat. No. 6,716,626; U.S. Patent Publication Nos. 2005/0176631, 2005/0037457, 2004/0185494, 2004/0259780, 2002/0164713, and 2001/0012628; WO 01/36640; WO 03/011213; WO 03/059270; WO 04/110472; WO 05/061712; WO 05/072769; WO 05/091944; WO 05/113606; WO 06/028595; WO 06/028714; WO 06/050247; WO 06/065582; WO 06/078463; WO01/018172; WO09/149171; WO10/042747; WO12/066075; WO11/154349; WO13/052311; WO13/188181, which are incorporated by reference in their entirety herein.

As used herein, the terms “variant FGF-21” and “variant FGF-21 polypeptide” refer to a FGF-21 polypeptide that differs from a reference wild-type FGF-21 polypeptide (e.g., a wild-type human FGF-21 of SEQ ID NO: 3) in at least one amino acid position and typically has at least one biological activity of a fibroblast growth factor 21, as well as FGF-21 analogs, FGF-21 isoforms, FGF-21 mimetics, FGF-21 fragments, hybrid FGF-21 proteins, fusion proteins, oligomers and multimers, homologues, glycosylation pattern variants, splice variants, and muteins thereof, regardless of the biological activity of the same. The term encompasses both naturally occurring and non-naturally occurring variants, e.g., a variant resulting from the substitution of an amino acid in a wild-type FGF-21 polypeptide, e.g., a polypeptide of SEQ ID NO:3, with a non-natural amino acid (e.g., para-acetyl-L-phenylalanine). The substitution can be, for example, the result of recombinant expression or chemical or enzymatic synthesis. In some aspects, a variant FGF-21 polypeptide of the present disclosure comprises, consists, or consists essentially of a polypeptide of SEQ ID NO: 1.

Variant FGF-21 polypeptides of the present disclosure encompass a FGF-21 polypeptide comprising one or more amino acid substitutions, additions or deletions. For example, a variant FGF-21 polypeptide of the present disclosure comprises one or more amino acid substitutions (for example with naturally occurring or non-naturally occurring amino acids), deletions (terminal or internal deletions), or modification such as the attachment of a heterologous moiety (C-terminal, N-terminal, or internal, either by intercalation/insertion in the amino acid sequence or by side-chain attachment). The term variant FGF-21 polypeptide also encompasses polymorphisms (e.g., naturally occurring FGF-21 sequence variants), e.g., the P-form or L-form of FGF-21.

Substitutions in a wide variety of amino acid positions in naturally-occurring FGF-21 polypeptide have been described. Substitutions including but not limited to, those that modulate solubility or stability, increase agonist activity, increase in vivo or in vitro half-life, increase protease resistance, convert the polypeptide into an antagonist, reduce immunogenicity or toxicity, facilitate purification or manufacturability, or any combination thereof, and are also encompassed by the term variant FGF-21 polypeptide.

The term variant FGF-21 polypeptide also includes biologically-active fragments, biologically active variants and stereoisomers of the naturally-occurring FGF-21 polypeptide as well as agonist, mimetic, and antagonist variants of the naturally-occurring FGF-21 and polypeptide fusions thereof. Fusions comprising additional amino acids at the amino terminus, carboxyl terminus, or both, are encompassed by the term variant FGF-21 polypeptide.

Exemplary fusions include, but are not limited to, e.g., methionyl FGF-21 in which a methionine is linked to the N-terminus of a FGF-21 polypeptide resulting, for example, from the recombinant expression of the mature form of FGF-21 lacking the leader or signal peptide or portion thereof (a methionine is linked to the N-terminus of FGF-21 resulting from the recombinant expression, e.g. in E. coli), fusions for the purpose of purification (including, but not limited to, to poly-histidine or affinity epitopes).

The term variant FGF-21 polypeptide also includes glycosylated FGF-21 polypeptides, such as but not limited to, polypeptides glycosylated at any amino acid position, N-linked or O-linked glycosylated forms of the polypeptide. Variants containing single nucleotide changes are also considered as biologically active variants of FGF-21. In addition, splice variants are also included.

The term variant FGF-21 polypeptide also includes FGF-21 heterodimers, homodimers, heteromultimers, or homomultimers of any one or more unmodified or modified FGF-21s or any other polypeptide, protein, carbohydrate, polymer, small molecule, linker, ligand, or other biologically active molecule of any type, linked by chemical means or expressed as a fusion protein, as well as polypeptide analogues containing, for example, specific deletions or other modifications yet maintain biological activity.

In some aspects, the variant FGF-21 polypeptide comprise an addition, substitution or deletion that increases the affinity of the FGF-21 polypeptide for its receptor. Similarly, the term variant FGF-21 polypeptide comprises chemically or enzymatically cleavage sequences, protease-cleaved sequences, reactive groups, antibody-binding domains (including but not limited to, FLAG or poly-His) or other affinity based sequences (including, but not limited to, FLAG, poly-His, GST, etc.) or linked molecules (including, but not limited to, biotin) that improve detection (including, but not limited to, GFP), purification, transport through tissues or cell membranes, prodrug release or activation, FGF-21 polypeptide size reduction, or other traits of the polypeptide.

In some aspects, the variant FGF-21 polypeptide comprises a polypeptide having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 1 or SEQ ID NO: 3 or SEQ ID NO: 1 or SEQ ID NO: 3 without Methione at the N terminus, wherein the polypeptide has a FGF-21 activity. In some aspects, the variant FGF-21 polypeptide comprises the amino acid sequence as set forth in SEQ ID NO: 5 or SEQ ID NO: 6.

In some aspects, the variant FGF-21 polypeptide consists or consists essentially of a polypeptide having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 1 or SEQ ID NO: 3, or SEQ ID NO: 1 or SEQ ID NO: 3 without Methione at the N terminus, wherein the polypeptide has a FGF-21 activity. In some aspects, the variant FGF-21 polypeptide consists essentially of the amino acid sequence as set forth in SEQ ID NO: 5 or SEQ ID NO: 6.

Variant FGF-21 polypeptides encompassed by this definition include, e.g., variant FGF-21 polypeptides comprising at least one non-natural amino acid. In some aspects, the non-natural amino acid is an amino acid which upon reaction with an aminooxy derivative can form a stable oxime linkage, e.g., p-acetylphenylalanine, m-acetylphenylalanine, p-(3-oxobutenoyl)-L-phenylalanine, p-(2-amino-3-hydroxyethyl)phenylalanine, and the like. In some aspects, the non-natural amino acid is p-acetylphenylalanine. In some aspects, the non-natural amino acid is p-acetyl-L-phenylalanine.

In some aspects, one or more non-natural amino acids are incorporated in one or more of the following positions of wild type FGF-21: before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 (i.e., at the carboxyl terminus of the protein) (amino acid positions corresponding to SEQ ID NO: 3).

In some specific aspects, a variant FGF-21 of the present disclosure is a modified FGF-21 polypeptide SEQ ID NO: 1, i.e., a derivative of the wild type FGF-21 of SEQ ID NO: 3 in which glutamine 109 of wild type FGF-21 has been substituted with a non-natural para-acetyl-L-phenylalanine amino acid.

In some specific aspects, the FGF-21 polypeptide of the present disclosure is modified such that methione at the N terminus of the FGF-21 polypeptide (SEQ ID NO: 1 or SEQ ID NO: 3) is removed: SEQ ID NO: 5 or 6.

As disclosed above, a variant FGF-21 polypeptide of the present disclosure can be linked to a PEG (polyethylene glycol) moiety. Linkage of PEG to a variant FGF-21 polypeptide disclosed herein (e.g., a FGF-21 polypeptide of SEQ ID NO: 1) can result in changes including, but not limited to, increased or modulated serum (in vivo) half-life, or increased or modulated therapeutic half-life relative to the unmodified form, modulated immunogenicity or toxicity, modulated physical association characteristics such as aggregation and multimer formation, altered receptor binding, altered binding to one or more binding partners, and altered receptor dimerization or multimerization. In some aspects, linkage of PEG to a variant FGF-21 disclosed herein (e.g., a FGF-21 polypeptide of SEQ ID NO: 1) improves or alters pharmacokinetic or biophysical properties including but not limited to increasing the rate of absorption, reducing toxicity, improving solubility, reducing protein aggregation, increasing biological activity and/or target selectivity of the PEGylated FGF-21, increasing manufacturability, and/or reducing immunogenicity (see, e.g., U.S. Pat. No. 4,179,337), compared to a reference compound such as an unconjugated form of the variant FGF-21 polypeptide (e.g., a FGF-21 polypeptide of SEQ ID NO:1) or wild-type FGF-21 (e.g., a FGF-21 polypeptide of SEQ ID NO:3). In some aspects, at least one linker can be interposed between the variant FGF-21 polypeptide moiety and the PEG moiety. In some aspects, the PEG moiety comprises a distal methoxy (—O—CH₃) group.

An examination of the crystal structure of FGF-21 or FGF family member(s) and its interaction with the FGF receptor can indicate which certain amino acid residues have side chains that are fully or partially accessible to solvent. The side chain of a non-natural amino acid at these positions may point away from the protein surface and out into the solvent and thus be linked to PEG.

PEG can be linked to one or more of the following amino acid positions of a wild type FGF-21 polypeptide or variant FGF-21 polypeptide: before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 (i.e., at the carboxyl terminus of the protein) (amino acid positions corresponding to SEQ ID NO: 3). In some aspects, the PEG is attached to a side chain of a non-natural amino acid, e.g., a phenylalanine derivative such as para-acetyl-L-phenylalanine, that substitutes a naturally occurring amino acid at any of the positions disclosed above.

PEGs of the present disclosure includes, but are not limited to, polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, discrete PEG, polypropylene oxide/ethylene oxide copolymer, polyalkylene glycol and derivatives thereof, copolymers of polyalkylene glycols and derivatives thereof, or mixtures thereof. In some aspects, the PEG may have a branched structure. Branched PEGs are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999).

In some aspects, the molecular weight of the PEG is about 30 kDa. Other sizes may be used, depending on the desired profile (e.g., the duration of sustained release desired, the effects, if any on biological activity, the ease in handling, the degree or lack of antigenicity and other known effects of the polyethylene glycol to a protein or analog). In some aspects, the molecular weight of the PEG is about 28 kDa, about 29 kDa, about 30 kDa, about 31 kDa, or about 32 kDa. In some aspects, the molecular weight of the PEG is between about 28 kDa and about 29 kDa, between about 29 kDa and about 30 kDa, between about 30 kDa and about 31 kDa, or between about 31 kDa and about 32 kDa. Methods of determining the molecular weight of PEG (as well as FGF-21 conjugates) are well known in the art. Any known methods, e.g., Mass Spectrometry (e.g., MALDI-TOF and/or ESI), can be used to measure the molecular weight of the PEG for the present disclosure.

In some aspects, the PEG has about 600 ethylene glycol units, about 610 ethylene glycol units, about 620 ethylene glycol units, about 630 ethylene glycol units, about 640 ethylene glycol units, about 650 ethylene glycol units, about 660 ethylene glycol units, about 670 ethylene glycol units, about 680 ethylene glycol units, about 690 ethylene glycol units, about 700 ethylene glycol units, about 710 ethylene glycol units, about 720 ethylene glycol units, about 730 ethylene glycol units, about 740 ethylene glycol units, about 750 ethylene glycol units, about 760 ethylene glycol units, about 770 ethylene glycol units, about 780 ethylene glycol units, about 790 ethylene glycol units, or about 800 ethylene glycol units.

In some aspects, the PEG has between about 600 ethylene glycol units and about 610 ethylene glycol units, between about 610 ethylene glycol units and about 620 ethylene glycol units, between about 620 ethylene glycol units and about 630 ethylene glycol units, between about 630 ethylene glycol units and about 640 ethylene glycol units, between about 640 ethylene glycol units and about 650 ethylene glycol units, between about 650 ethylene glycol units and about 660 ethylene glycol units, between about 660 ethylene glycol units and about 670 ethylene glycol units, between about 670 ethylene glycol units and about 680 ethylene glycol units, between about 680 ethylene glycol units and about 690 ethylene glycol units, between about 690 ethylene glycol units and about 700 ethylene glycol units, between about 700 ethylene glycol units and about 710 ethylene glycol units, between about 710 ethylene glycol units and about 720 ethylene glycol units, between about 720 ethylene glycol units and about 730 ethylene glycol units, between about 730 ethylene glycol units and about 740 ethylene glycol units, between about 740 ethylene glycol units and about 750 ethylene glycol units, between about 750 ethylene glycol units and about 760 ethylene glycol units, between about 760 ethylene glycol units and about 770 ethylene glycol units, between about 770 ethylene glycol units and about 780 ethylene glycol units, between about 780 ethylene glycol units and about 790 ethylene glycol units, or between about 790 ethylene glycol units and about 800 ethylene glycol units.

In some aspects, the PEG has 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or 700 ethylene glycol units.

In some specific aspects, the PEG moiety is linked to a variant FGF-21 polypeptide of the present disclosure (e.g., a FGF-21 polypeptide of SEQ ID NO:1) via an oxime linkage. In some aspects, the PEG moiety is linked to a variant FGF-21 polypeptide of the present disclosure (e.g., a variant FGF-21 polypeptide of SEQ ID NO: 1) via an oxime linkage formed between a reactive group of a PEG molecule (e.g., the aminooxy group of a 30 kDa methyl PEG₆₈₁ aminooxy molecule as presented in FIG. 7) and a reactive group of a non-natural amino acid in the variant FGF-21 polypeptide (e.g., the acetyl group of p-acetyl-phenylalanine, e.g., at amino acid position 109 of the sequence of the variant FGF-21 polypeptide). In some aspects, the non-natural amino acid is para-acetyl-L-phenylalanine replacing Gln109 of SEQ ID NO: 3. In some aspects, the PEG moiety comprises a distal methoxy (—O—CH₃) group.

In some aspects, the FGF-21 conjugate in a formulation disclosed herein is a PEG-FGF-21 of SEQ ID NO: 2, i.e., the FGF-21 of SEQ ID NO: 1 in which glutamine 109 of wild type FGF-21 (SEQ ID NO:3) has been replaced with para-acetyl-L-phenylalanine, and a PEG moiety, e.g., a linear PEG with molecular weight between about 28 kDa and about 32 kDa, e.g, about 30 kDa, which has been covalently attached to the para-acetyl-L-phenylalanine via an oxime linkage. In some aspects, the PEG moiety comprises a distal methoxy (—O—CH₃) group.

In some aspects, the FGF-21 conjugate in a formulation disclosed herein is a PEG-FGF-21 of SEQ ID NO: 2, i.e., an FGF-21 conjugate comprising (i) the FGF-21 of SEQ ID NO: 1 in which para-acetyl-L-phenylalanine replaces glutamine 109 of wild type FGF-21 (SEQ ID NO:3), and (ii) a PEG moiety, e.g., a linear PEG with molecular weight between about 28 kDa and about 32 kDa, e.g, about 30 kDa, covalently attached to the para-acetyl-L-phenylalanine via an oxime linkage. In some aspects, the PEG moiety comprises a distal methoxy (—O—CH₃) group.

In some aspects, the FGF-21 conjugate in a formulation disclosed herein is a PEG-FGF-21 of SEQ ID NO: 4, i.e., the FGF-21 of SEQ ID NO: 1 in which glutamine 109 of wild type FGF-21 (SEQ ID NO:3) has been replaced with para-acetyl-L-phenylalanine, and a PEG moiety comprising 681 ethylene glycol units has been covalently attached to the para-acetyl-L-phenylalanine via an oxime linkage. In some aspects, the PEG moiety comprises a distal methoxy (—O—CH₃) group.

In some aspects, the FGF-21 conjugate in a formulation disclosed herein is a PEG-FGF-21 of SEQ ID NO: 4, i.e., an FGF-21 conjugate comprising (i) the FGF-21 of SEQ ID NO: 1 in which para-acetyl-L-phenylalanine replaced glutamine 109 of wild type FGF-21 (SEQ ID NO:3), and (ii) a PEG moiety comprising 681 ethylene glycol units covalently attached to the para-acetyl-L-phenylalanine via an oxime linkage. In some aspects, the PEG moiety comprises a distal methoxy (—O—CH₃) group.

Thus, in some specific aspects of the present disclosure, the FGF-21 conjugate in a formulation disclosed herein comprises (i) a FGF-21 polypeptide moiety of SEQ ID NO: 1, and (ii) a PEG moiety with 681 ethylene glycol units, i.e., a PEG moiety with a molecular weight of approximately 30 kDa, wherein the PEG moiety is covalently attached to the para-acetyl-L-phenylalanine at position 109 via an oxime linkage. In some aspects, the PEG moiety comprises a distal methoxy (—O—CH₃) group.

In some aspects, the oxime linkage is formed by chemical reaction between a reactive group of a non-natural amino acid present in a variant FGF-21 (e.g., the acetyl group of p-acetyl-phenylalanine) and a reactive group of a PEG molecule (e.g., the aminooxy group of a 30 kDa PEG₆₈₁ aminooxy molecule comprising a distal methoxy group as presented in FIG. 7). In some aspects, the non-natural amino acid can be incorporated into the variant FGF-21 polypeptide recombinantly (e.g., by expression in a prokaryotic cell culture), using in vitro transcription and translation, using chemical synthesis, or using any methods known in the art. In some aspects, the PEG molecule can be chemically linked to an amino acid (e.g., p-acetyl-phenylalanine), and the PEG-amino acid subsequently incorporated into a FGF-21 polypeptide, for example, via chemical synthesis. In some aspects, the PEG molecule comprises a distal methoxy (—O—CH₃) group.

Production of FGF-21 polypeptides via yeast expression has been hindered in the past by the presence of O-linked glycosylation, which required site-specific mutagenesis to remove O-linked glycosylation sites. FGF-21 polypeptides also show a substantial degree of glycosylation when produced recombinantly in mammalian cell cultures. Thus, in some aspects, the variant FGF-21 polypeptides of the present disclosure are produced recombinantly in prokaryotic cells cultures. Accordingly, in some aspects, the variant FGF-21 polypeptides of the present disclosure are not glycosylated.

As disclosed above, the present disclosure provided pharmaceutical formulations comprising a FGF-21 conjugate disclosed herein, e.g., a PEG-FGF-21 of SEQ ID NO: 2 or 5 or SEQ ID NO:4 or 6, and an aminopolycarboxylic acid cation chelator, e.g., diethylenetriaminepentaacetic acid (DTPA). The terms “chelator” or “cation chelator” are interchangeable and refer to any substance that is able to remove a metal ion from a solution system by forming a new complex ion that has different chemical properties than those of the original metal ion. In particular, the cation chelators disclosed herein are chelators that specifically bind divalent metals, e.g., Ca++.

In some aspects, the FGF-21 conjugate formulations disclosed herein exhibit one or more improvements in stability, e.g., a lower rate of deamidation, and/or a lower rate of aggregation. For example, the inclusion of an aminopolycarboxylic acid cation chelator such as DTPA in the FGF-21 conjugate formulation can lower the rate of deamidation of a FGF-21 conjugate disclosed herein, e.g., a PEG-FGF-21 of SEQ ID NO: 2 or 5 or SEQ ID NO:4 or 6, when stored at a certain temperature (e.g., 40° C.) for a certain period of time (e.g., 1 month) with respect to the reference formulation.

In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or 5 or SEQ ID NO:4 or 6, in a pharmaceutical formulation stored at about 25° C., at about 30° C., at about 35° C., at about 40° C., or at about 45° C. with respect to the reference formulation. In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or 5 or SEQ ID NO:4 or 6, in a pharmaceutical formulation stored at a temperature above 25° C., above 30° C., above 35° C., about 40° C., or about 45° C. with respect to the reference formulation.

In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or SEQ ID NO:4, in a pharmaceutical formulation stored between about 20° C. and about 25° C., about 25° C. and about 30° C., about 30° C. and about 35° C., or about 40° C. and about 45° C. with respect to the reference formulation.

In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or SEQ ID NO:4, in a pharmaceutical formulation at a temperature or temperature range disclosed above after storage for about 1 week, about 2 weeks, about 3 weeks, about 1 month, about 2 months, about 3 months, or about 4 months with respect to the reference formulation.

In a specific aspect, the aminopolycarboxylic acid cation chelator (e.g., DTPA) can lower the rate of deamidation of a FGF-21 conjugate disclosed herein, e.g., a PEG-FGF-21 of SEQ ID NO: 2 or SEQ ID NO:4, in a pharmaceutical formulation stored at 40° C. for about a month with respect to the reference formulation.

In some aspects, the aminopolycarboxylic acid cation chelator (e.g., DTPA) can lower the rate of high molecular weight (HMW) aggregation of a FGF-21 conjugate disclosed herein, e.g., a PEG-FGF-21 of SEQ ID NO: 2 or SEQ ID NO:4, in a pharmaceutical formulation stored at about 25° C., at about 30° C., at about 35° C., at about 40° C., or at about 45° C. with respect to the reference formulation. In some aspects, the aminopolycarboxylic acid cation chelator (e.g., DTPA) can lower the rate of HMW aggregation of a FGF-21 conjugate disclosed herein, e.g., a PEG-FGF-21 of SEQ ID NO: 2 or SEQ ID NO:4, in a pharmaceutical formulation stored at a temperature above 25° C., above 30° C., above 35° C., about 40° C., or about 45° C. with respect to the reference formulation.

In some aspects, the aminopolycarboxylic acid cation chelator (e.g., DTPA) can lower the rate of HMW aggregation of a FGF-21 conjugate disclosed herein, e.g., a PEG-FGF-21 of SEQ ID NO: 2 or SEQ ID NO:4, in a pharmaceutical formulation stored between about 20° C. and about 25° C., about 25° C. and about 30° C., about 30° C. and about 35° C., or about 40° C. and about 45° C. with respect to the reference formulation.

In some aspects, the aminopolycarboxylic acid cation chelator (e.g., DTPA) can lower the rate of HMW aggregation of a FGF-21 conjugate disclosed herein, e.g., a PEG-FGF-21 of SEQ ID NO: 2 or 5 or SEQ ID NO:4 or 6, in a pharmaceutical formulation at a temperature or temperature range disclosed above after storage for about 1 week, about 2 weeks, about 3 weeks, about 1 month, about 2 months, about 3 months, or about 4 months with respect to the reference formulation. In a specific aspect, the aminopolycarboxylic acid cation chelator (e.g., DTPA) can lower the rate of HMW aggregation of a FGF-21 conjugate disclosed herein, e.g., a PEG-FGF-21 of SEQ ID NO: 2 or SEQ ID NO:4, in a pharmaceutical formulation stored at 40° C. for about a month with respect to the reference formulation.

In some aspects, the aminopolycarboxylic acid cation chelator, e.g., DTPA, prevents or mitigates oxidation of one or more amino acids, e.g., methionines, in a FGF-21 conjugate disclosed herein, e.g., a PEG-FGF-21 of SEQ ID NO: 2 or SEQ ID NO:4. In particular aspects, the aminopolycarboxylic acid cation chelator, e.g., DTPA, prevents or mitigates oxidation of amino acid 1 and/or amino acid 169 of SEQ ID NO: 3 (or the corresponding amino acids in SEQ ID NOS: 1, 2 or 4) at 25° C. and/or 40° C.

In some specific aspects, the aminopolycarboxylic acid cation chelator is DTPA. Pentetic acid or diethylenetriaminepentaacetic acid (DTPA) is an aminopolycarboxylic acid consisting of a diethylenetriamine backbone with five carboxymethyl groups. The conjugate base of DTPA has a high affinity for metal cations. Thus, the penta-anion DTPA⁵⁻ is potentially an octadentate ligand assuming that each nitrogen centre and each COO⁻-group counts as a centre for coordination. The formation constants for its complexes are about 100 greater than those for EDTA.

As a chelating agent, DTPA wraps around a metal ion by forming up to eight bonds. Transition metals, however, usually form less than eight coordination bonds. So, after forming a complex with a metal, DTPA still has the ability to bind to other reagents, as is shown by its derivative pendetide. For example, in its complex with copper(II), DTPA binds in a hexadentate manner utilizing the three amine centres and three of the five carboxylates.

In some other aspects, the aminopolycarboxylic acid cation chelator can be another aminopolycarboxylic acid cation chelator, such as ethylenediaminetetraacetic acid (EDTA), ethylene glycol-bis(β-aminoethyl ether)-N,N,N′,N′-tetraacetic acid (EGTA), 1,4,7,10-tetraazacyclododecane-1,4,7,10-tetraacetic acid (DOTA), or related compound, e.g., tiuxetan (a modified version of DTPA whose carbon backbone contains an isothiocyanatobenzyl and a methyl group). Other chelating agents related to DTPA and EDTA known in the art are those in which the nitrogens of the amide groups may be substituted by one or more C₁₋₁₈ alkyl groups, e.g. DTPA.BMA and EDTA.BMA.

In some aspects, the DTPA cation chelator is present in an amount between about 10 μM and about 100 μM, between 15 μM and about 95 μM, between about 20 μM and about 90 μM, between about 25 μM and about 85 μM, between about 30 μM and about 80 μM, between about 35 μM and about 75 μM, between about 40 μM and about 70 μM, between about 45 μM and about 65 μM, between about 50 μM and about 60 μM, between about 25 μM and about 75 μM, between about 40 μM and about 60 μM, between about 30 μM and about 70 μM, or between about 40 μM and about 75 μM.

In some aspects, the DTPA cation chelator is present in an amount of about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM or about 100 μM.

In some aspects, the DTPA cation chelator is present in an amount of at least about 15 μM, at least about 20 μM, at least about 25 μM, at least about 30 μM, at least about 35 μM, at least about 40 μM, at least about 45 μM, at least about 50 μM, at least about 55 μM, at least about 60 μM, at least about 65 μM, at least about 70 μM, or at least about 75 μM.

In a specific aspects, the aminopolycarboxylic acid cation chelator, e.g., DTPA, is present in an amount of 50 μM.

In some aspects, the pH of a formulation disclosed herein is above about 6.5, above about 6.6, above about 6.7, above about 6.8, above about 6.9, above about 7.0, above about 7.1, above about 7.2, above about 7.3, above about 7.4, or above about 7.5. In some aspects, the pH of the formulation is above 6.5, above 6.6, above 6.7, above 6.8, above 6.9, above 7.0, above 7.1, above 7.2, above 7.3, above 7.4, or above 7.5. In some aspects, the pH of the formulation is about 6.5, about 6.6, about 6.7, about 6.8, about 6.9, about 7.0, about 7.1, about 7.2, about 7.3, about 7.4, or about 7.5.

In some aspects, the pH of the formulation is between about 6.5 and about 7.5, about 6.6 and about 7.5, about 6.7 and about 7.5, about 6.8 and about 7.5, about 6.9 and about 7.5, about 7.0 and about 7.5, about 7.1 and about 7.5, about 7.2 and about 7.5, about 7.3 and about 7.5, about 7.4 and about 7.5, about 6.5 and about 7.4, about 6.5 and about 7.3, about 6.5 and about 7.2, about 6.5 and about 7.1, about 6.5 and about 7.0, about 6.5 and about 6.9, about 6.5 and about 6.8, about 6.5 and about 6.7, about 6.6 and about 7.4, about 6.7 and about 7.4, about 6.8 and about 7.4, about 6.9 and about 7.4, about 7.0 and about 7.4, about 7.1 and about 7.4, about 7.2 and about 7.4, about 7.3 and about 7.4, about 6.5 and about 7.3, about 6.6 and about 7.3, about 6.7 and about 7.3, about 6.7 and about 7.3, about 6.8 and about 7.3, about 6.9 and about 7.3, about 7.0 and about 7.3, about 7.1 and about 7.3, about 7.2 and about 7.3, about 6.5 and about 7.2, about 6.6 and about 7.2, about 6.7 and about 7.2, about 6.8 and about 7.2, about 6.9 and about 7.2, about 7.0 and about 7.2, about 7.1 and about 7.2, about 6.9 and about 7.1, or about 7.0 and about 7.1.

In some aspects, the pH of the formulation is about 6.8, about 6.9, about 7.0, about 7.1, about 7.2, about 7.3, about 7.4, or about 7.5. In some aspects, the pharmaceutical formulation is more stable than a reference formulation with a pH of 6.5.

In some aspects, pharmaceutical formulation further comprises a surfactant. The term “surfactant” as used herein means any compound, typically an amphipathic molecule, that reduces surface tension when dissolved or suspended in water or water solutions, or which reduces interfacial tension between two liquids, or between a liquid and a solid.

In the context of the present disclosure, a surfactant is any compound that decreases interfacial stress and shear in a solution comprising a FGF-21 conjugate disclosed herein, e.g., a PEG-FGF-21 of SEQ ID NO: 2 or 5 or SEQ ID NO:4 or 6.

In some aspects, the surfactant is a nonionic surfactant, i.e., is a surfactant that tends to have no net charge in neutral solutions. In some aspects, the nonanionic surfactant is a polysorbate. Polysorbates are an important class of non-ionic surfactants used widely in protein pharmaceuticals to stabilize the proteins against interface-induced aggregation and to minimize surface adsorption of proteins (Wang W 2005. Protein aggregation and its inhibition in biopharmaceutics. Int J Pharm 289 (1-2):1-30). Polysorbates are amphiphilic, non-ionic surfactants composed of fatty acid esters of polyoxyethylene (POE) sorbitan. Commercially available polysorbates are chemically diverse mixtures containing mainly sorbitan POE fatty acid esters.

As used herein, the term “polysorbate” refers to oleate esters of sorbitol and its anhydrides, typically copolymerized with ethylene oxide. Exemplary polysorbates include Polysorbate 20 (TWEEN 20; PS20) (polyoxyethylene (20) sorbitan monolaurate); Polysorbate 40 (TWEEN 40; PS40) (polyoxyethylene (20) sorbitan monopalmitate); Polysorbate 60 (TWEEN 60; PS60) (polyoxyethylene (20) sorbitan monostearate); and Polysorbate 80 (TWEEN 80; PS80) (polyoxyethylene (20) sorbitan monooleate).

The number 20 following the ‘polyoxyethylene’ part refers to the total number of oxyethylene —(CH₂CH₂O)— groups found in the molecule. The number following the ‘polysorbate’ part is related to the type of fatty acid associated with the polyoxyethylene sorbitan part of the molecule. Monolaurate is indicated by 20, monopalmitate is indicated by 40, monostearate by 60, and monooleate by 80. In some aspects, the non-ionic surfactant is present in an amount above the critical micelle concentration (CMC), which for polyoxyethylene sorbitan fatty acid esters is approximately an amount of at least 0.01 mg/ml. See Wan and Lee, Journal of Pharm Sci, 63, p.136, 1974. Surfactant concentrations (%) throughout the present specification correspond to (w/v).

In some aspects, the polysorbate is polysorbate 80 (PS80). In some aspects, the polysorbate 80 surfactant is present in an amount of about 0.01% to about 0.1% (w/v), about 0.02% to about 0.1% (w/v), about 0.03% to about 0.1% (w/v), about 0.04% to about 0.1% (w/v), about 0.05% to about 0.1% (w/v), about 0.06% to about 0.1% (w/v), about 0.07% to about 0.1% (w/v), about 0.08% to about 0.1% (w/v), about 0.09% to about 0.1% (w/v), about 0.02% to about 0.09% (w/v), about 0.03% to about 0.09% (w/v), about 0.04% to about 0.09% (w/v), about 0.05% to about 0.09% (w/v), about 0.06% to about 0.09% (w/v), about 0.07% to about 0.09% (w/v), about 0.08% to about 0.09% (w/v), about 0.03% to about 0.08% (w/v), about 0.04% to about 0.08% (w/v), about 0.05% to about 0.08% (w/v), about 0.06% to about 0.08% (w/v), about 0.07% to about 0.08% (w/v), about 0.04% to about 0.07% (w/v), about 0.05% to about 0.07% (w/v), about 0.06% to about 0.07% (w/v), or about 0.05% to about 0.06% (w/v).

In some aspects, the polysorbate 80 surfactant is present in an amount of at least about 0.01% (w/v), at least about 0.02% (w/v), at least about 0.03% (w/v), at least about 0.04% (w/v), at least about 0.05% (w/v), at least about 0.06% (w/v), at least about 0.07% (w/v), at least about 0.08% (w/v), at least about 0.09% (w/v) or at least about 0.1% (w/v).

In some aspects, the surfactant, e.g., polysorbate 80, mitigates particulate and/or air bubble formation, e.g., when the formulation agitated on a shaker. In some aspects, the presence of the surfactant, e.g., polysorbate 80, in the formulation can reduce particulate formation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% compared to the level of particulate formation in a reference formulation. In some aspects, the presence of the surfactant, e.g., polysorbate 80, in the formulation can reduce air bubble formation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% compared to the level of bubble formation in a reference formulation.

In some aspects, the pharmaceutical formulation further comprises an amino acid buffering agent. Amino acids may be advantageously used as buffers in pharmaceutical applications because they naturally present substances which are easily metabolizable. Furthermore, amino acids used as buffers can also protect proteins in the amorphous phase if the formulation is freeze-dried. A suitable amino acid buffer can contain histidine, lysine, and/or arginine. Histidine has a good buffering capacity around pH 7.

As used herein, the term “histidine” comprises either L-histidine or D-histidine, a solvated form of histidine, a hydrated form (e.g., monohydrate) of histidine, or an anhydrous form of histidine, or a mixture thereof. Other suitable buffers in the formulations of the present disclosure glutamate, Tris, or succinate, to mention just a few.

In some specific aspects, the amino acid buffering agent is L-histidine.

In some aspects, the histidine buffering agent is present in an amount of about 10 mM to about 100 mM histidine, about 15 mM to about 100 mM histidine, about 20 mM to about 100 mM histidine, about 25 mM to about 100 mM, about 30 mM to about 100 mM histidine, about 35 mM to about 100 mM histidine, about 40 mM to about 100 mM histidine, about 45 mM to about 100 mM histidine, about 50 mM to about 100 mM histidine, about 55 mM to about 100 mM histidine, about 60 mM to about 100 mM histidine, about 65 mM to about 100 mM histidine, about 70 mM to about 100 mM histidine, about 75 mM to about 100 mM histidine, about 80 mM to about 100 mM histidine, about 85 mM to about 100 mM histidine, about 90 mM to about 100 mM histidine, or about 95 mM to about 100 mM histidine.

In some aspects, the histidine buffering agent is present in an amount of about 20 mM to about 90 mM histidine, about 25 mM to about 90 mM histidine, about 30 mM to about 90 mM histidine, about 35 mM to about 90 mM histidine, about 40 mM to about 90 mM histidine, about 45 mM to about 90 mM histidine, about 50 mM to about 90 mM histidine, about 55 mM to about 90 mM histidine, about 60 mM to about 90 mM histidine, about 65 mM to about 90 mM histidine, about 70 mM to about 90 mM histidine, about 75 mM to about 90 mM histidine, about 80 mM to about 90 mM histidine, about 85 mM to about 90 mM histidine, about 30 mM to about 80 mM histidine, about 35 mM to about 80 mM histidine, about 40 mM to about 80 mM histidine, about 45 mM to about 80 mM histidine.

In some aspects, the histidine buffering agent is present in an amount of about 50 mM to about 80 mM histidine, about 55 mM to about 80 mM histidine, about 60 mM to about 80 mM histidine, about 65 mM to about 80 mM histidine, about 70 mM to about 80 mM histidine, about 75 mM to about 80 mM histidine, about 40 mM to about 70 mM histidine, about 45 mM to about 70 mM histidine, about 50 mM to about 70 mM histidine, about 55 mM to about 70 mM histidine, about 60 mM to about 70 mM histidine, about 65 mM to about 70 mM histidine, about 10 mM to about 30 mM histidine, about 15 mM to about 30 mM histidine, about 20 mM to about 30 mM histidine, about 25 mM to about 30 mM histidine, about 15 mM to about 25 mM histidine, or about 20 mM to about 25 mM.

In some aspects, the histidine buffering agent is present in an amount of about 15 mM to about 20 mM histidine, about 40 mM to about 60 mM histidine, about 45 mM to about 60 mM histidine, about 50 mM to about 60 mM histidine, about 15.5 mM to about 24.5 mM histidine, about 16 mM to about 24 mM histidine, about 16.5 mM to about 23.5 mM histidine, about 17 mM to about 23 mM histidine, about 17.5 mM to about 22.5 mM histidine, about 18 mM to about 22 mM histidine, about 18.5 mM to about 21.5 mM histidine, about 19 mM to about 21 mM histidine, or about 19.5 mM to about 20.5 mM histidine.

In some aspects, the histidine buffering agent is present in an amount of about 10 mM histidine, about 11 mM histidine, about 12 mM histidine, about 13 mM histidine, about 14 mM histidine, about 15 mM histidine, about 16 mM histidine, about 17 mM histidine, about 18 mM histidine, about 19 mM histidine, about 20 mM histidine, about 21 mM histidine, about 22 mM histidine, about 23 mM histidine, about 24 mM histidine, about 25 mM histidine, about 26 mM histidine, about 27 mM histidine, about 28 mM histidine, about 29 mM histidine, about 30 mM histidine, about 31 mM histidine, about 32 mM histidine, about 33 mM histidine, about 34 mM histidine, about 35 mM histidine, about 36 mM histidine, about 37 mM histidine, about 38 mM histidine, about 39 mM histidine, about 40 mM histidine, about 41 mM histidine, about 42 mM histidine, about 43 mM histidine, about 44 mM histidine, about 45 mM histidine, about 46 mM histidine, about 47 mM histidine, about 48 mM histidine, about 49 mM histidine, or about 50 mM histidine.

In some aspects, the pharmaceutical formulation further comprises an osmotic regulator (also known in the art tonicity agents). According to the present disclosure the osmotic regulator (tonicity agent) can comprises a polyol, a saccharide, a carbohydrate, a salt, such as sodium chloride, or mixtures thereof. Exemplary polyols comprise those with a molecular weight that is less than about 600 kD (e.g., in the range from 120 to 400 kD), e.g., mannitol, trehalose, sorbitol, erythritol, isomalt, lactitol, maltitol, xylitol, glycerol, lactitol, propylene glycol, polyethylene glycol, inositol, or mixtures thereof.

Saccharide or carbohydrate osmotic regulators comprise monosaccharides, disaccharides and polysaccharides or mixtures thereof. In some aspects, the saccharide or carbohydrate is selected from the group consisting of fructose, glucose, mannose, sucrose, sorbose, xylose, lactose, maltose, sucrose, dextran, pullulan, dextrin, cyclodextrins, soluble starch, hydroxyethyl starch, water-soluble glucans, and mixtures thereof.

In some aspects, the osmotic regulator comprises a saccharide selected from the group of reducing sugar or non reducing sugar or mixtures thereof. In some aspects, the osmotic regulator the tonicity agent comprises a saccharide which is a non-reducing sugar, preferably a sugar selected from the group consisting of sucrose, trehalose, and mixtures thereof. In some specific aspects, the non-reducing sugar is sucrose.

In some aspects, the sucrose osmotic regulator is present in an amount of about 100 mM to about 1 M sucrose, about 200 mM to about 1 M sucrose, about 300 mM to about 1 M sucrose, about 400 mM to about 1 M sucrose, about 500 mM to about 1 M sucrose, about 600 mM to about 1 M sucrose, about 700 mM to about 1 M sucrose, about 800 mM to about 1 M sucrose, about 900 mM to about 1 M sucrose, about 200 mM to about 900 mM sucrose, about 300 mM to about 900 mM sucrose, about 400 mM to about 900 mM sucrose, about 500 mM to about 900 mM sucrose, about 600 mM to about 900 mM sucrose, about 700 mM to about 900 mM sucrose, about 800 mM to about 900 mM sucrose, about 300 mM to about 800 mM sucrose, about 400 mM to about 800 mM sucrose, about 500 mM to about 800 mM sucrose, about 600 mM to about 800 mM sucrose, about 700 mM to about 800 mM sucrose, about 400 mM to about 700 mM sucrose, about 500 mM to about 700 mM sucrose, about 600 mM to about 700 mM sucrose, or about 500 mM to about 600 mM sucrose.

In some aspects, the sucrose osmotic regulator is present in an amount of about 100 mM sucrose, about 150 mM sucrose, about 200 mM sucrose, about 250 mM sucrose, about 300 mM sucrose, about 350 mM sucrose, about 400 mM sucrose, about 450 mM sucrose, about 500 mM sucrose, about 550 mM sucrose, about 600 mM sucrose, about 650 mM sucrose, about 700 mM sucrose, about 750 mM sucrose, about 800 mM sucrose, about 850 mM sucrose, about 900 mM sucrose, about 950 mM sucrose, or about 1M sucrose.

In some aspects, a FGF-21 conjugate disclosed herein, e.g., a PEG-FGF-21 of SEQ ID NO: 2 or 5 or SEQ ID NO: 4 or 6, is present at a concentration between about 1 mg/ml and about 40 mg/ml. In some aspects, a FGF-21 conjugate disclosed herein, e.g., a PEG-FGF-21 of SEQ ID NO: 2 or 5 or SEQ ID NO: 4 or 6, is present at a concentration of about 10 mg/ml, about 20 mg/ml, about 30 mg/ml, or about 40 mg/ml. In some aspects, the concentration of the FGF-21 conjugate in the pharmaceutical formulation disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

In some aspects, a FGF-21 conjugate disclosed herein, e.g., a PEG-FGF-21 of SEQ ID NO: 2 or SEQ ID NO:4, is present at a concentration between about 1 mg/ml and 5 mg/ml, between about 5 mg/ml and about 10 mg/ml, between about 10 mg/ml and about 15 mg/ml, between about 15 mg/ml and about 20 mg/ml, between about 20 mg/ml and about 25 mg/ml, between about 25 mg/ml and about 30 mg/ml, between about 30 mg/ml and about 35 mg/ml, or between about 35 mg/ml and about 40 mg/ml.

In some aspects, a FGF-21 conjugate disclosed herein, e.g., a PEG-FGF-21 of SEQ ID NO: 2 or SEQ ID NO:4, is present at a concentration of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, or about 40 mg/ml. In a specific aspect, a FGF-21 conjugate disclosed herein, e.g., a PEG-FGF-21 of SEQ ID NO: 2 or SEQ ID NO:4 is present at a concentration of about 10 mg/ml. In a specific aspect, a FGF-21 conjugate disclosed herein, e.g., a PEG-FGF-21 of SEQ ID NO: 2 or SEQ ID NO:4 is present at a concentration of about 20 mg/ml.

In some aspects, the concentration of a FGF-21 conjugate disclosed herein, e.g., a PEG-FGF-21 of SEQ ID NO: 2 or SEQ ID NO:4, is determined according to methods known in the art, or the specific methods disclosed in the Examples section of the present specification.

In some aspects a FGF-21 conjugate disclosed herein, e.g., a PEG-FGF-21 of SEQ ID NO: 2 or SEQ ID NO:4 is present in a formulation disclosed herein in an amount between about 1 mg and about 40 mg per dose. In some aspects, a FGF-21 conjugate disclosed herein, e.g., a PEG-FGF-21 of SEQ ID NO: 2 or SEQ ID NO:4 is present in a formulation disclosed herein in an amount between about 1 mg and about 5 mg per dose, between about 5 mg and about 10 mg per dose, or between about 10 mg and about 15 mg per dose, between about 15 mg and about 20 mg per dose, or between about 20 mg and about 25 mg per dose, between about 25 mg and about 30 mg per dose, or between about 30 mg and about 35 mg per dose, or between about 35 mg and about 40 mg per dose. In some aspects, the dose is a flat dose. In some aspects, the amount of the FGF-21 conjugate in the pharmaceutical formulation disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

In some aspects, a FGF-21 conjugate disclosed herein, e.g., a PEG-FGF-21 of SEQ ID NO: 2 or SEQ ID NO:4, is present in a formulation disclosed herein in an amount between about 19 mg and about 21 mg per dose, between about 18 mg and about 22 mg per dose, between about 17 mg and about 23 mg per dose, between about 16 mg and about 24 mg per dose, between about 15 mg and about 25 mg per dose, between about 14 mg and about 26 mg per dose, between about 13 mg and about 27 mg per dose, between about 12 mg and about 28 mg per dose, between about 11 mg and about 29 mg per dose, or between about 10 mg and about 30 mg per dose. In some aspects, a FGF-21 conjugate disclosed herein, e.g., a PEG-FGF-21 of SEQ ID NO: 2 or SEQ ID NO:4 is present in a formulation disclosed herein in an amount higher than 40 mg per dose. In some aspects, the dose is a flat dose.

In some aspects, a FGF-21 conjugate disclosed herein, e.g., a PEG-FGF-21 of SEQ ID NO: 2 or SEQ ID NO:4 is present in a formulation disclosed herein in an amount of about 1 mg per dose, about 2 mg per dose, about 3 mg per dose, about 4 mg per dose, about 5 mg per dose, about 6 mg per dose, about 7 mg per dose, about 8 mg per dose, about 9 mg per dose, about 10 mg per dose, about 11 mg per dose, about 12 mg per dose, about 13 mg per dose, about 14 mg per dose, about 15 mg per dose, about 16 mg per dose, about 17 mg per dose, about 18 mg per dose, about 19 mg per dose, about 20 mg per dose, about 21 mg per dose, about 22 mg per dose, about 23 mg per dose, about 24 mg per dose, about 25 mg per dose, about 26 mg per dose, about 27 mg per dose, about 28 mg per dose, about 29 mg per dose, about 30 mg per dose, about 31 mg per dose, about 32 mg per dose, about 33 mg per dose, about 34 mg per dose, about 35 mg per dose, about 36 mg per dose, about 37 mg per dose, about 38 mg per dose, about 39 mg per dose, or about 40 mg per dose. In a specific aspects, a FGF-21 conjugate disclosed herein, e.g., a PEG-FGF-21 of SEQ ID NO: 2 or SEQ ID NO:4 is present in a formulation disclosed herein in an amount of about 10 mg per dose. In a specific aspects, a FGF-21 conjugate disclosed herein, e.g., a PEG-FGF-21 of SEQ ID NO: 2 or SEQ ID NO:4 is present in a formulation disclosed herein in an amount of about 20 mg per dose. In some aspects, the dose is a flat dose.

In some aspects, the pharmaceutical formulation is formulated for subcutaneous administration. As discussed below, other the pharmaceutical formulations disclosed herein can be administered via other routes. In some aspects, the pharmaceutical formulation is formulated for subcutaneous administration, e.g., with a safety syringe. In some aspects, the formulation is formulated for daily or weekly administration, for example, every 1, 2, 3, 4, 5, 6, days, every week, or every two weeks. In some aspects, the formulation is an aqueous formulation.

In some aspects, the present disclosure provides a pharmaceutical formulation comprising (i) a FGF-21 conjugate disclosed herein, e.g., a PEG-FGF-21 of SEQ ID NO: 2 or SEQ ID NO:4; (ii) histidine at a concentration between about 10 mM and about 50 mM; (iii) sucrose at a concentration between about 100 mM and about 1M; (iv) Polysorbate 80 at a concentration between about 0.01% and about 0.1% (w/v); and, (v) DTPA at a concentration between about 10 μM and about 100 μM; wherein the pH of the formulation is between about 6.7 and about 7.5.

In some aspects, the present disclosure provides a pharmaceutical formulation comprising (i) a FGF-21 conjugate disclosed herein, e.g., a PEG-FGF-21 of SEQ ID NO: 5 or SEQ ID NO:6; (ii) histidine at a concentration between about 10 mM and about 50 mM; (iii) sucrose at a concentration between about 100 mM and about 1M; (iv) Polysorbate 80 at a concentration between about 0.01% and about 0.1% (w/v); and, (v) DTPA at a concentration between about 10 μM and about 100 μM; wherein the pH of the formulation is between about 6.7 and about 7.5.

Also provided is a pharmaceutical formulation comprising (i) a FGF-21 conjugate disclosed herein, e.g., a PEG-FGF-21 of SEQ ID NO: 2 or 5 or SEQ ID NO:4 or 6; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 μM; wherein the pH is about 7.1.

Also provided is a pharmaceutical formulation comprising (i) a FGF-21 conjugate disclosed herein, e.g., a PEG-FGF-21 of SEQ ID NO: 2 or 5 or SEQ ID NO:4 or 6; (ii) histidine at a concentration of 20 mM; (iii) sucrose at a concentration of 600 mM; (iv) Polysorbate 80 at a concentration of 0.05% (w/v); and (v) DTPA at a concentration of 50 μM; wherein the pH is 7.1.

Also provided is a pharmaceutical formulation comprising (i) a FGF-21 conjugate disclosed herein, e.g., a PEG-FGF-21 of SEQ ID NO: 2 or 5 or SEQ ID NO:4 or 6; (ii) histidine at a concentration of about 20 mM; and (iii) sucrose at a concentration of about 600 mM; wherein the pH is about 7.0.

Also provided is a pharmaceutical formulation comprising (i) a FGF-21 conjugate disclosed herein, e.g., a PEG-FGF-21 of SEQ ID NO: 2 or 5 or SEQ ID NO:4 or 6; (ii) histidine at a concentration of 20 mM; and (iii) sucrose at a concentration of 600 mM; wherein the pH is 7.0.

In some specific aspects, the present disclosure provide a pharmaceutical formulation comprising (i) a FGF-21 conjugate disclosed herein, e.g., a PEG-FGF-21 of SEQ ID NO: 2 or 5 or SEQ ID NO:4 or 6, at a concentration of about 10 mg/mL; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 uM; wherein the pH is about 7.1. In some aspects, the concentration of the FGF-21 conjugate in the pharmaceutical formulation disclosed herein is measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

Also provided is a pharmaceutical formulation comprising (i) a FGF-21 conjugate disclosed herein, e.g., a PEG-FGF-21 of SEQ ID NO: 2 or 5 or SEQ ID NO:4 or 6, at a concentration of about 20 mg/mL; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 uM; wherein the pH is about 7.1. In some aspects, the concentration of the FGF-21 conjugate in the pharmaceutical formulation disclosed herein is measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

Also provided is a pharmaceutical formulation comprising (i) a FGF-21 conjugate disclosed herein, e.g., a PEG-FGF-21 of SEQ ID NO: 2 or 5 or SEQ ID NO:4 or 6, at a concentration of 10 mg/mL; (ii) histidine at a concentration of 20 mM; (iii) sucrose at a concentration of 600 mM; (iv) Polysorbate 80 at a concentration of 0.05% (w/v); and (v) DTPA at a concentration of 50 uM; wherein the pH is 7.1.

Also provided is a pharmaceutical formulation comprising (i) a FGF-21 conjugate disclosed herein, e.g., a PEG-FGF-21 of SEQ ID NO: 2 or 5 or SEQ ID NO:4 or 6, at a concentration of 20 mg/mL; (ii) histidine at a concentration of 20 mM; (iii) sucrose at a concentration of 600 mM; (iv) Polysorbate 80 at a concentration of 0.05% (w/v); and (v) DTPA at a concentration of 50 uM; wherein the pH is 7.1.

The present disclosure also provides pharmaceutical formulation prepared according to any of the methods to improve the stability of a formulation comprising a FGF-21 conjugate disclosed herein, e.g., a PEG-FGF-21 of SEQ ID NO: 2 or 5 or SEQ ID NO:4 or 6.

In a specific aspect, the present disclosure provides a pharmaceutical formulation comprising a FGF-21 conjugate of SEQ ID NO:4 or 6 at a concentration of 20 g/L in 20 mM histidine, 600 mM sucrose, 50 uM DTPA, and 0.05% PS80, pH 7.1. In a specific aspect, the present disclosure provides a pharmaceutical formulation comprising a FGF-21 conjugate of SEQ ID NO:4 or 6 at a concentration of 10 g/L in 20 mM histidine, 600 mM sucrose, 50 uM DTPA, and 0.05% PS80 (w/v), pH 7.1. In some aspects, the concentration of the FGF-21 conjugate in the pharmaceutical formulation disclosed herein is measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

In some aspects, the formulation is frozen. In some aspects, the formulation is stored in a bag, e.g., a clam shell bag. In some aspects, the bag, e.g., a clam shell bag, has a volume between 6 L and 12 L.

In some aspects, the formulation is contained in a vial. In some aspects, the formulation is contained in a syringe. In some aspects, the syringe is a safety syringe. In some aspects, the syringe is a pre-fillable syringe. In some aspects, the syringe is a BD NEOPAK™ pre-fillable syringe. In some aspects, the formulation is contained in a self-injection device.

In some aspects, the syringe (e.g., pre-fillable syringe) or self-injection device comprises 1 mL of a pharmaceutical formulation comprising a FGF-21 conjugate of SEQ ID NO:4 or 6 at a concentration of 10 g/L in 20 mM histidine, 600 mM sucrose, 50 uM DTPA, and 0.05% PS80 (w/v), pH 7.1. In some aspects, the syringe (e.g., pre-fillable syringe) or self-injection device comprises 1 mL of a pharmaceutical formulation comprising a FGF-21 conjugate of SEQ ID NO:4 or 6 at a concentration of 20 g/L in 20 mM histidine, 600 mM sucrose, 50 uM DTPA, and 0.05% PS80, pH 7.1. In some aspects, the concentration of the FGF-21 conjugate in the pharmaceutical formulation disclosed herein is measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

### II. Methods of Manufacture

The present disclosure also provides methods to improve or enhance the stability of a pharmaceutical formulation comprising a FGF-21 conjugate disclosed herein, e.g., a PEG-FGF-21 of SEQ ID NO: 2 or 5 or SEQ ID NO:4 or 6, comprising admixing an aminopolycarboxylic acid cation chelator, e.g., DTPA, wherein the formulation has improved stability compared to a reference formulation that does not contain the aminopolycarboxylic acid cation chelator. These methods of stabilization comprise (i) the admixture of an aminopolycarboxylic acid cation chelator to the formulation, (ii) the admixture of a polysorbate surfactant, e.g., polysorbate 80, (iii) adjusting the pH of the formulation to approximately 7.1, or (iv) any combination thereof.

In some aspects, a pharmaceutical formulation described herein is made by the process of admixing (i) a FGF-21 conjugate disclosed herein, e.g., a PEG-FGF-21 of SEQ ID NO: 2 or 5 or SEQ ID NO:4 or 6, in amount to achieve a final concentration of about 10 mg/mL; (ii) histidine in amount to achieve a final concentration of about 20 mM; (iii) sucrose in amount to achieve a final a concentration of about 600 mM; (iv) Polysorbate 80 in amount to achieve a final concentration of about 0.05% (w/v); and (v) DTPA in amount to achieve a final concentration of about 50 uM; and adjust the pH at about 7.1.

In some aspects, a pharmaceutical formulation described herein is made by the process of admixing (i) a FGF-21 conjugate disclosed herein, e.g., a PEG-FGF-21 of SEQ ID NO: 2 or 5 or SEQ ID NO:4 or 6, in amount to achieve a final concentration of about 20 mg/mL; (ii) histidine in amount to achieve a final concentration of about 20 mM; (iii) sucrose in amount to achieve a final a concentration of about 600 mM; (iv) Polysorbate 80 in amount to achieve a final concentration of about 0.05% (w/v); and (v) DTPA in amount to achieve a final concentration of about 50 uM; and adjust the pH at about 7.1.

In some aspects, a pharmaceutical formulation described herein is made by the process of admixing (i) a FGF-21 conjugate disclosed herein, e.g., a PEG-FGF-21 of SEQ ID NO: 2 or 5 or SEQ ID NO:4 or 6, in amount to achieve a final concentration of 10 mg/mL; (ii) histidine in amount to achieve a final concentration of 20 mM; (iii) sucrose in amount to achieve a final a concentration of 600 mM; (iv) Polysorbate 80 in amount to achieve a final concentration of 0.05% (w/v); and (v) DTPA in amount to achieve a final concentration of 50 uM; and adjust the pH at 7.1.

In some aspects, a pharmaceutical formulation described herein is made by the process of admixing (i) a FGF-21 conjugate disclosed herein, e.g., a PEG-FGF-21 of SEQ ID NO: 2 or 5 or SEQ ID NO:4 or 6, in amount to achieve a final concentration of 20 mg/mL; (ii) histidine in amount to achieve a final concentration of 20 mM; (iii) sucrose in amount to achieve a final a concentration of 600 mM; (iv) Polysorbate 80 in amount to achieve a final concentration of 0.05% (w/v); and (v) DTPA in amount to achieve a final concentration of 50 uM; and adjust the pH at 7.1.

As used herein the term “admixing” refers to the combination of the components of the formulations disclosed herein in no predetermined order to reach the disclosed concentrations by any means known in the art. For example, the excipients in a pharmaceutical formulation disclosed herein can be sequentially or simultaneously added to a solution comprising a FGF-21 conjugate disclosed herein, e.g., a PEG-FGF-21 of SEQ ID NO: 2 or SEQ ID NO:4. Alternatively, a concentrated solution comprising a FGF-21 conjugate disclosed herein, e.g., a PEG-FGF-21 of SEQ ID NO: 2 or SEQ ID NO:4, can be added to a solution comprising all or part of the excipients in the formulation. In other aspects, the excipients can be incorporated to the formulation using, e.g., dialysis or filtration.

In some aspects, the improvements in stability resulting from the application of the disclosed method comprise, for example, (i) an increase on physical stability with respect to a reference formulation, (ii) an increase in chemical stability with respect to a reference formulation, or (iii) a combination thereof.

In some aspects, the increase in physical stability comprises (i) prevention or decrease of FGF-21 polypeptide aggregation, (ii) prevention or decrease of FGF-21 polypeptide fragmentation, or (iii) a combination thereof.

In some aspects, the FGF-21 polypeptide aggregation observed in a pharmaceutical composition disclosed herein is about 90%, about 85%, about 80%, about 75%, about 70%, about 65%, about 60%, about 55%, about 50%, about 45%, about 40%, about 35%, about 30%, about 25%, about 20%, about 15%, or about 10% of the FGF-21 polypeptide aggregation observed in a reference formulation.

In some aspects, the FGF-21 polypeptide aggregation observed in a pharmaceutical composition disclosed herein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the FGF-21 polypeptide aggregation observed in a reference formulation.

In some aspects, the FGF-21 polypeptide fragmentation observed in a pharmaceutical composition disclosed herein is about 90%, about 85%, about 80%, about 75%, about 70%, about 65%, about 60%, about 55%, about 50%, about 45%, about 40%, about 35%, about 30%, about 25%, about 20%, about 15%, or about 10% of the FGF-21 polypeptide fragmentation observed in a reference formulation.

In some aspects, the FGF-21 polypeptide fragmentation observed in a pharmaceutical composition disclosed herein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the FGF-21 polypeptide fragmentation observed in a reference formulation.

In some aspects, the increase in chemical stability comprises (i) prevention or decrease of FGF-21 polypeptide deamidation, (ii) prevention or decrease of FGF-21 polypeptide oxidation, or (iii) a combination thereof. In some aspects, the FGF-21 polypeptide deamidation observed in a pharmaceutical composition disclosed herein is about 90%, about 85%, about 80%, about 75%, about 70%, about 65%, about 60%, about 55%, about 50%, about 45%, about 40%, about 35%, about 30%, about 25%, about 20%, about 15%, or about 10% of the FGF-21 polypeptide deamidation observed in a reference formulation.

In some aspects, the FGF-21 polypeptide deamidation observed in a pharmaceutical composition disclosed herein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the FGF-21 polypeptide deamidation observed in a reference formulation.

In some aspects, the FGF-21 polypeptide oxidation observed in a pharmaceutical composition disclosed herein is about 90%, about 85%, about 80%, about 75%, about 70%, about 65%, about 60%, about 55%, about 50%, about 45%, about 40%, about 35%, about 30%, about 25%, about 20%, about 15%, or about 10% of the FGF-21 polypeptide oxidation observed in a reference formulation.

In some aspects, the FGF-21 polypeptide oxidation observed in a pharmaceutical composition disclosed herein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the FGF-21 polypeptide oxidation observed in a reference formulation.

In some aspects, the methods to stabilize (improve or enhance the stability) a formulation disclosed herein can lower not only the level of degradation but also the rate of degradation: for example, the disclosed methods can lower the rate of polypeptide deamidation, lower the rate of FGF-21 polypeptide oxidation, lower the rate of FGF-21 polypeptide aggregation, lower the rate of FGF-21 polypeptide proteolytic degradation, or any combination thereof, with respected to a reference formulation.

For example, the admixture of an aminopolycarboxylic acid cation chelator such as DTPA in a pharmaceutical formulation disclosed herein can lower the rate of deamidation of a FGF-21 conjugate disclosed herein, e.g., a PEG-FGF-21 of SEQ ID NO: 2 or SEQ ID NO:4, when stored at a certain temperature for a certain period of time with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of deamidation of the FGF-21 conjugate when the pharmaceutical formulation stored, for example, at about 25° C., at about 30° C., at about 35° C., at about 40° C., or at about 45° C. with respect to the reference formulation.

In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or SEQ ID NO:4, when the pharmaceutical formulation is stored at a temperature above 25° C., above 30° C., above 35° C., about 40° C., or about 45° C. with respect to the reference formulation.

In some aspects, the aminopolycarboxylic acid cation chelator (e.g., DTPA) can lower the rate of deamidation of a FGF-21 conjugate disclosed herein, e.g., a PEG-FGF-21 of SEQ ID NO: 2 or SEQ ID NO:4, when the pharmaceutical formulation is stored between about 20° C. and about 25° C., about 25° C. and about 30° C., about 30° C. and about 35° C., or about 40° C. and about 45° C. with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of deamidation of the FGF-21 conjugate when the pharmaceutical formulation is stored at a temperature or temperature range disclosed above for about 1 week, about 2 weeks, about 3 weeks, about 1 month, about 2 months, about 3 months, or about 4 months with respect to the reference formulation.

In a specific aspect, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of deamidation of the FGF-21 conjugate when the pharmaceutical formulation is stored at 40° C. for about a month with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of high molecular weight (HMW) aggregation of the FGF-21 conjugate when the pharmaceutical formulation is stored at about 25° C., at about 30° C., at about 35° C., at about 40° C., or at about 45° C. with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of HMW aggregation of the FGF-21 conjugate when the pharmaceutical formulation is stored at a temperature above 25° C., above 30° C., above 35° C., about 40° C., or about 45° C. with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of HMW aggregation of the FGF-21 conjugate when the pharmaceutical formulation is stored between at a temperature about 20° C. and about 25° C., about 25° C. and about 30° C., about 30° C. and about 35° C., or about 40° C. and about 45° C. with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of HMW aggregation of the FGF-21 conjugate when the pharmaceutical formulation is stored at a temperature or temperature range disclosed above for about 1 week, about 2 weeks, about 3 weeks, about 1 month, about 2 months, about 3 months, or about 4 months with respect to the reference formulation.

In a specific aspect, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can lower the rate of HMW aggregation of the FGF-21 conjugate when the pharmaceutical formulation is stored at 40° C. for about a month with respect to the reference formulation.

In some aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can prevent or mitigate the oxidation of one or more methionines in the FGF-21 conjugate.

In particular aspects, the admixture of an aminopolycarboxylic acid cation chelator (e.g., DTPA) to a pharmaceutical formulation comprising a FGF-21 conjugate disclosed herein, e.g., a PEG-FGF-21 of SEQ ID NO: 2 or SEQ ID NO:4, can prevent or mitigate the oxidation of amino acid 1 and/or amino acid 169 of SEQ ID NO: 3 (wild type FGF-21) or the corresponding amino acids in SEQ ID NOS:1, 2, 4 or any other FGF-21 conjugate according to the present disclosure), e.g., at 25° C. and/or at 40° C.

The disclosed methods can use the aminopolycarboxylic acid cation chelator DTPA. However, in some other aspects, the aminopolycarboxylic acid cation chelator can be, e.g., another aminopolycarboxylic acid cation chelator such as EDTA, EGTA, DOTA, a DTPA-related compound such as tiuxetan, or any chelating agents related to DTPA and EDTA known in the art, e.g. DTPA.BMA and EDTA.BMA.

In some aspects, the DTPA cation chelator can be admixed to an amount between about 10 μM and about 100 μM, between 15 μM and about 95 μM, between about 20 μM and about 90 μM, between about 25 μM and about 85 μM, between about 30 μM and about 80 μM, between about 35 μM and about 75 μM, between about 40 μM and about 70 μM, between about 45 μM and about 65 μM, between about 50 μM and about 60 μM, between about 25 μM and about 75 μM, between about 40 μM and about 60 μM, between about 30 μM and about 70 μM, or between about 40 μM and about 75 μM.

In some aspects, the DTPA cation chelator can be admixed to an amount of about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM or about 100 μM.

In some aspects, the DTPA cation chelator can be admixed to an amount of at least about 15 μM, at least about 20 μM, at least about 25 μM, at least about 30 μM, at least about 35 μM, at least about 40 μM, at least about 45 μM, at least about 50 μM, at least about 55 μM, at least about 60 μM, at least about 65 μM, at least about 70 μM, or at least about 75 μM.

In a specific aspect, the aminopolycarboxylic acid cation chelator, e.g., DTPA, is admixed to an amount of 50 μM.

In some aspects of the methods disclosed herein, the pH of the formulation is adjusted to a pH above about 6.5, above about 6.6, above about 6.7, above about 6.8, above about 6.9, above about 7.0, above about 7.1, above about 7.2, above about 7.3, above about 7.4, or above about 7.5.

In some aspects, the pH of the formulation is adjusted to a pH above 6.5, above 6.6, above 6.7, above 6.8, above 6.9, above 7.0, above 7.1, above 7.2, above 7.3, above 7.4, or above 7.5.

In some aspects, the pH of the formulation is adjusted to a pH of 6.5, 6.6, 6.7, 6.8, 6.9, 7.0, 7.1, 7.2, 7.3, 7.4, or 7.5.

In some aspects, the pH of the formulation is adjusted to a pH between about 6.5 and about 7.5, about 6.6 and about 7.5, about 6.7 and about 7.5, about 6.8 and about 7.5, about 6.9 and about 7.5, about 7.0 and about 7.5, about 7.1 and about 7.5, about 7.2 and about 7.5, about 7.3 and about 7.5, about 7.4 and about 7.5, about 6.5 and about 7.4, about 6.5 and about 7.3, about 6.5 and about 7.2, about 6.5 and about 7.1, about 6.5 and about 7.0, about 6.5 and about 6.9, about 6.5 and about 6.8, about 6.5 and about 6.7, about 6.6 and about 7.4, about 6.7 and about 7.4, about 6.8 and about 7.4, about 6.9 and about 7.4, about 7.0 and about 7.4, about 7.1 and about 7.4, about 7.2 and about 7.4, about 7.3 and about 7.4, about 6.5 and about 7.3, about 6.6 and about 7.3, about 6.7 and about 7.3, about 6.7 and about 7.3, about 6.8 and about 7.3, about 6.9 and about 7.3, about 7.0 and about 7.3, about 7.1 and about 7.3, about 7.2 and about 7.3, about 6.5 and about 7.2, about 6.6 and about 7.2, about 6.7 and about 7.2, about 6.8 and about 7.2, about 6.9 and about 7.2, about 7.0 and about 7.2, about 7.1 and about 7.2, about 6.9 and about 7.1, or about 7.0 and about 7.1,

In some aspects, the pH of the formulation is adjusted to a pH of about 6.8, about 6.9, about 7.0, about 7.1, about 7.2, about 7.3, about 7.4, or about 7.5. In some aspects, the pharmaceutical formulation, after adjusting the pH, is more stable than a reference formulation with a pH of 6.5.

In some aspects, the pH of the formulation is adjusted to a pH of 6.8, 6.9, 7.0, 7.1, 7.2, 7.3, 7.4, or 7.5. In some aspects, the pharmaceutical formulation, after adjusting the pH, is more stable than a reference formulation with a pH of 6.5.

In some aspects, the method to improve the stability of a pharmaceutical formulation comprising a FGF-21 conjugate disclosed herein, e.g., a PEG-FGF-21 of SEQ ID NO: 2 or SEQ ID NO:4, further comprises admixing a surfactant. In some aspects, the admixed surfactant is a nonionic surfactant, i.e., a surfactant that tends to have no net charge in neutral solutions. In some aspects, the admixed nonanionic surfactant is a polysorbate. In some aspects, the non-ionic surfactant is admixed in an amount above the critical micelle concentration (CMC), which for polyoxyethylene sorbitan fatty acid esters is approximately an amount of at least 0.01 mg/ml. See Wan and Lee, Journal of Pharm Sci, 63, p.136, 1974. In some aspects of the present methods, the polysorbate is polysorbate 80 (PS80).

In some aspects, the PS80 surfactant is admixed to the pharmaceutical formulation in an amount of about 0.01% to about 0.1% (w/v), about 0.02% to about 0.1% (w/v), about 0.03% to about 0.1% (w/v), about 0.04% to about 0.1% (w/v), about 0.05% to about 0.1% (w/v), about 0.06% to about 0.1% (w/v), about 0.07% to about 0.1% (w/v), about 0.08% to about 0.1% (w/v), about 0.09% to about 0.1% (w/v), about 0.02% to about 0.09% (w/v), about 0.03% to about 0.09% (w/v), about 0.04% to about 0.09% (w/v), about 0.05% to about 0.09% (w/v), about 0.06% to about 0.09% (w/v), about 0.07% to about 0.09% (w/v), about 0.08% to about 0.09% (w/v), about 0.03% to about 0.08% (w/v), about 0.04% to about 0.08% (w/v), about 0.05% to about 0.08% (w/v), about 0.06% to about 0.08% (w/v), about 0.07% to about 0.08% (w/v), about 0.04% to about 0.07% (w/v), about 0.05% to about 0.07% (w/v), about 0.06% to about 0.07% (w/v), or about 0.05% to about 0.06% (w/v).

In some aspects, the polysorbate 80 surfactant is admixed in an amount of at least about 0.01% (w/v), at least about 0.02% (w/v), at least about 0.03% (w/v), at least about 0.04% (w/v), at least about 0.05% (w/v), at least about 0.06% (w/v), at least about 0.07% (w/v), at least about 0.08% (w/v), at least about 0.09% (w/v) or at least about 0.1% (w/v). In some aspects, the surfactant, e.g., PS80 is admixed in an amount sufficient to mitigates or prevent particulate formation and/or air bubble formation, e.g., when the formulation agitated (for example, on a shaker).

In some aspects, the method to improve the stability of a pharmaceutical formulation comprising a FGF-21 conjugate disclosed herein, e.g., a PEG-FGF-21 of SEQ ID NO: 2 or SEQ ID NO:4, further comprises admixing an amino acid buffering agent, e.g., histidine (i.e., L-histidine, D-histidine, a solvated histidine, a hydrated histidine, an anhydrous histidine, or a mixture thereof).

In some aspects, the histidine buffering agent is admixed in an amount of about 10 mM to about 100 mM histidine, about 15 mM to about 100 mM histidine, about 20 mM to about 100 mM histidine, about 25 mM to about 100 mM histidine, about 30 mM to about 100 mM histidine, about 35 mM to about 100 mM histidine, about 40 mM to about 100 mM histidine, about 45 mM to about 100 mM histidine, about 50 mM to about 100 mM histidine, about 55 mM to about 100 mM histidine, about 60 mM to about 100 mM histidine, about 65 mM to about 100 mM histidine, about 70 mM to about 100 mM histidine, about 75 mM to about 100 mM histidine, about 80 mM to about 100 mM histidine, about 85 mM to about 100 mM histidine, about 90 mM to about 100 mM histidine, about 95 mM to about 100 mM histidine, about 20 mM to about 90 mM histidine, about 25 mM to about 90 mM histidine, about 30 mM to about 90 mM histidine, about 35 mM to about 90 mM histidine, about 40 mM to about 90 mM histidine, about 45 mM to about 90 mM histidine, about 50 mM to about 90 mM histidine, about 55 mM to about 90 mM histidine, about 60 mM to about 90 mM histidine, about 65 mM to about 90 mM histidine, about 70 mM to about 90 mM histidine, about 75 mM to about 90 mM histidine, about 80 mM to about 90 mM histidine, about 85 mM to about 90 mM histidine, about 30 mM to about 80 mM histidine, about 35 mM to about 80 mM histidine, about 40 mM to about 80 mM histidine, about 45 mM to about 80 mM histidine, about 50 mM to about 80 mM histidine, about 55 mM to about 80 mM histidine, about 60 mM to about 80 mM histidine, about 65 mM to about 80 mM histidine, about 70 mM to about 80 mM histidine, about 75 mM to about 80 mM histidine, about 40 mM to about 70 mM histidine, about 45 mM to about 70 mM histidine, about 50 mM to about 70 mM histidine, about 55 mM to about 70 mM histidine, about 60 mM to about 70 mM histidine, about 65 mM to about 70 mM histidine, about 10 mM to about 30 mM histidine, about 15 mM to about 30 mM histidine, about 20 mM to about 30 mM histidine, about 25 mM to about 30 mM histidine, about 15 mM to about 25 mM histidine, about 20 mM to about 25 mM, about 15 mM to about 20 mM histidine, about 40 mM to about 60 mM histidine, about 45 mM to about 60 mM histidine, about 50 mM to about 60 mM histidine, about 15.5 mM to about 24.5 mM histidine, about 16 mM to about 24 mM histidine, about 16.5 mM to about 23.5 mM histidine, about 17 mM to about 23 mM histidine, about 17.5 mM to about 22.5 mM histidine, about 18 mM to about 22 mM histidine, about 18.5 mM to about 21.5 mM histidine, about 19 mM to about 21 mM histidine, or about 19.5 mM to about 20.5 mM histidine.

In some aspects, the histidine buffering agent is admixed in an amount of about 10 mM histidine, about 11 mM histidine, about 12 mM histidine, about 13 mM histidine, about 14 mM histidine, about 15 mM histidine, about 16 mM histidine, about 17 mM histidine, about 18 mM histidine, about 19 mM histidine, about 20 mM histidine, about 21 mM histidine, about 22 mM histidine, about 23 mM histidine, about 24 mM histidine, about 25 mM histidine, about 26 mM histidine, about 27 mM histidine, about 28 mM histidine, about 29 mM histidine, about 30 mM histidine, about 31 mM histidine, about 32 mM histidine, about 33 mM histidine, about 34 mM histidine, about 35 mM histidine, about 36 mM histidine, about 37 mM histidine, about 38 mM histidine, about 39 mM histidine, about 40 mM histidine, about 41 mM histidine, about 42 mM histidine, about 43 mM histidine, about 44 mM histidine, about 45 mM histidine, about 46 mM histidine, about 47 mM histidine, about 48 mM histidine, about 49 mM histidine, or about 50 mM histidine.

In some aspects, the method to improve the stability of a pharmaceutical formulation comprising a FGF-21 conjugate disclosed herein, e.g., a PEG-FGF-21 of SEQ ID NO: 2 or SEQ ID NO:4, further comprises admixing an osmotic regulator (tonicity agent). According to the present disclosure the osmotic regulator (tonicity agent) can comprises a polyol, a saccharide, a carbohydrate, a salt, such as sodium chloride, or mixtures thereof. Exemplary polyols comprise those with a molecular weight that is less than about 600 kD (e.g., in the range from 120 to 400 kD), e.g., mannitol, trehalose, sorbitol, erythritol, isomalt, lactitol, maltitol, xylitol, glycerol, lactitol, propylene glycol, polyethylene glycol, inositol, or mixtures thereof. Saccharide or carbohydrate osmotic regulators comprise monosaccharides, disaccharides and polysaccharides or mixtures thereof.

In some aspects, the saccharide or carbohydrate is selected from the group consisting of fructose, glucose, mannose, sucrose, sorbose, xylose, lactose, maltose, sucrose, dextran, pullulan, dextrin, cyclodextrins, soluble starch, hydroxyethyl starch, water-soluble glucans, and mixtures thereof. In some aspects, the osmotic regulator comprises a saccharide selected from the group of reducing sugar or non reducing sugar or mixtures thereof. In some aspects, the osmotic regulator the tonicity agent comprises a saccharide which is a non-reducing sugar, preferably selected from the group consisting of sucrose, trehalose, and mixtures thereof. In some specific aspects, the non-reducing sugar is sucrose.

In some aspects, the sucrose osmotic regulator is admixed in an amount of about 100 mM to about 1 M sucrose, about 200 mM to about 1 M sucrose, about 300 mM to about 1 M sucrose, about 400 mM to about 1 M sucrose, about 500 mM to about 1 M sucrose, about 600 mM to about 1 M sucrose, about 700 mM to about 1 M sucrose, about 800 mM to about 1 M sucrose, about 900 mM to about 1 M sucrose, about 200 mM to about 900 mM sucrose, about 300 mM to about 900 mM sucrose, about 400 mM to about 900 mM sucrose, about 500 mM to about 900 mM sucrose, about 600 mM to about 900 mM sucrose, about 700 mM to about 900 mM sucrose, about 800 mM to about 900 mM sucrose, about 300 mM to about 800 mM sucrose, about 400 mM to about 800 mM sucrose, about 500 mM to about 800 mM sucrose, about 600 mM to about 800 mM sucrose, about 700 mM to about 800 mM sucrose, about 400 mM to about 700 mM sucrose, about 500 mM to about 700 mM sucrose, about 600 mM to about 700 mM sucrose, or about 500 mM to about 600 mM sucrose.

In some aspects, the sucrose osmotic regulator is admixed in an amount of about 100 mM sucrose, about 150 mM sucrose, about 200 mM sucrose, about 250 mM sucrose, about 300 mM sucrose, about 350 mM sucrose, about 400 mM sucrose, about 450 mM sucrose, about 500 mM sucrose, about 550 mM sucrose, about 600 mM sucrose, about 650 mM sucrose, about 700 mM sucrose, about 750 mM sucrose, about 800 mM sucrose, about 850 mM sucrose, about 900 mM sucrose, about 950 mM sucrose, or about 1M sucrose.

In some aspects, the FGF-21 conjugate useful in the methods disclosed herein comprises an FGF-21 polypeptide having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 3 (wild type human FGF-21), wherein the FGF-21 polypeptide has a FGF-21 activity.

In some aspects, the FGF-21 conjugate is a PEG-FGF-21 of SEQ ID NO: 2, i.e., the FGF-21 of SEQ ID NO: 1 in which glutamine 109 of wild type FGF-21 (SEQ ID NO:3) has been replaced with para-acetyl-L-phenylalanine and a PEG moiety, e.g., a linear PEG with molecular weight between about 28 kDa and about 32 kDa, e.g, about 30 kDa, which has been covalently attached to the para-acetyl-L-phenylalanine via an oxime linkage. In some aspects, the FGF-21 conjugate is a PEG-FGF-21 of SEQ ID NO: 4, i.e., the FGF-21 of SEQ ID NO: 1 in which glutamine 109 of wild type FGF-21 (SEQ ID NO:3) has been replaced with para-acetyl-L-phenylalanine, and a PEG moiety comprising 681 ethylene glycol units has been covalently attached to the para-acetyl-L-phenylalanine via an oxime linkage.

In some aspects of the methods disclosed herein, the FGF-21 conjugate, e.g., a PEG-FGF-21 of SEQ ID NO: 2 or SEQ ID NO:4, is present at a concentration between about 1 mg/ml and about 40 mg/ml. In some aspects, the FGF-21 conjugate disclosed herein, e.g., a PEG-FGF-21 of SEQ ID NO: 2 or SEQ ID NO:4, is present at a concentration of about 10 mg/ml, about 20 mg/ml, about 30 mg/ml, or about 40 mg/ml. In some specific aspects, the FGF-21 conjugate disclosed herein, e.g., a PEG-FGF-21 of SEQ ID NO: 2 or SEQ ID NO:4, is present at a concentration of about 10 mg/ml. In some specific aspects, the FGF-21 conjugate disclosed herein, e.g., a PEG-FGF-21 of SEQ ID NO: 2 or SEQ ID NO:4, is present at a concentration of about 20 mg/ml.

In some aspects, the formulation comprises about 1 mL of a FGF-21 conjugate disclosed herein, e.g., a PEG-FGF-21 of SEQ ID NO: 2 or SEQ ID NO:4, at a concentration of about 10 mg/ml. In some aspects, the formulation comprises about 1 mL of a FGF-21 conjugate disclosed herein, e.g., a PEG-FGF-21 of SEQ ID NO: 2 or SEQ ID NO:4, at a concentration of about 20 mg/ml.

In some aspects, the concentration of FGF-21 conjugate disclosed herein, e.g., a PEG-FGF-21 of SEQ ID NO: 2 or SEQ ID NO:4, is determined according to methods known in the art, or the specific methods disclosed in the Examples section of the instant specification.

In some aspects of the methods disclosed herein, the formulation is formulated for subcutaneous administration. In some aspects, the formulation is formulated for subcutaneous administration with a safety syringe. In some aspects, the formulation is formulated for daily or weekly administration. In some aspects, the formulation is an aqueous formulation.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration between about 10 mM and about 50 mM; (ii) sucrose at a concentration between about 100 mM and about 1M; (iii) Polysorbate 80 at a concentration between about 0.01% and about 0.1% (w/v); and, (iv) DTPA at a concentration between about 10 μM and about 100 μM; wherein the pH of the formulation is between about 6.7 and about 7.5.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration between about 15 mM and about 45 mM; (ii) sucrose at a concentration between about 200 mM and about 900 mM; (iii) Polysorbate 80 at a concentration between about 0.02% and about 0.09% (w/v); and, (iv) DTPA at a concentration between about 20 μM and about 90 μM; wherein the pH of the formulation is between about 6.8 and about 7.4.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration between about 15 mM and about 40 mM; (ii) sucrose at a concentration between about 300 mM and about 800 mM; (iii) Polysorbate 80 at a concentration between about 0.03% and about 0.08% (w/v); and, (iv) DTPA at a concentration between about 30 μM and about 80 μM; wherein the pH of the formulation is between about 6.9 and about 7.3.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration between about 15 mM and about 30 mM; (ii) sucrose at a concentration between about 400 mM and about 800 mM; (iii) Polysorbate 80 at a concentration between about 0.04% and about 0.07% (w/v); and, (iv) DTPA at a concentration between about 40 μM and about 70 μM; wherein the pH of the formulation is between about 7 and about 7.2.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration between about 15 mM and about 25 mM; (ii) sucrose at a concentration between about 500 mM and about 700 mM; (iii) Polysorbate 80 at a concentration between about 0.04% and about 0.06% (w/v); and, (iv) DTPA at a concentration between about 45 μM and about 55 μM; wherein the pH of the formulation is between about 7 and about 7.1. In some aspects, the concentration of the FGF-21 conjugate in the pharmaceutical formulation disclosed herein is measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)).

The present disclosure also provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 μM; wherein the pH is about 7.1.

The present disclosure also provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 μM; wherein the pH is 7.1.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration of about 20 mM; and (ii) sucrose at a concentration of about 600 mM; wherein the pH is about 7.0.

The present disclosure provides a method to improve the stability of a formulation comprising a FGF-21 conjugate disclosed herein, e.g., a PEG-FGF-21 of SEQ ID NO: 2 or SEQ ID NO:4 (e.g., at a concentration of 10 mg/mL or 20 mg/mL), comprising admixing (i) histidine at a concentration of 20 mM; and (ii) sucrose at a concentration of 600 mM; wherein the pH is 7.0.

The present disclosure provides a method to improve the stability of a formulation comprising a FGF-21 conjugate disclosed herein, e.g., a PEG-FGF-21 of SEQ ID NO: 2 or SEQ ID NO:4, at a concentration of about 10 mg/mL comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 uM; wherein the pH is about 7.1.

The present disclosure provides a method to improve the stability of a formulation comprising a FGF-21 conjugate disclosed herein, e.g., a PEG-FGF-21 of SEQ ID NO: 2 or SEQ ID NO:4, at a concentration of about 20 mg/mL comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 uM; wherein the pH is about 7.1.

The present disclosure provides a method to improve the stability of a formulation comprising a FGF-21 conjugate disclosed herein, e.g., a PEG-FGF-21 of SEQ ID NO: 2 or SEQ ID NO:4, at a concentration of about 10 mg/mL comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 uM; wherein the pH is 7.1.

The present disclosure provides a method to improve the stability of a formulation comprising a FGF-21 conjugate disclosed herein, e.g., a PEG-FGF-21 of SEQ ID NO: 2 or SEQ ID NO:4, at a concentration of about 20 mg/mL comprising admixing comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 uM; wherein the pH is 7.1.

The present disclosure also provides a method to improve the stability of a formulation comprising a FGF-21 conjugate disclosed herein, e.g., a PEG-FGF-21 of SEQ ID NO: 5 or SEQ ID NO:6 (e.g., at a concentration of 10 mg/mL or 20 mg/mL), comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 μM; wherein the pH is about 7.1.

The present disclosure also provides a method to improve the stability of a formulation comprising a FGF-21 conjugate disclosed herein, e.g., a PEG-FGF-21 of SEQ ID NO: 5 or SEQ ID NO:6 (e.g., at a concentration of 10 mg/mL or 20 mg/mL), comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 μM; wherein the pH is 7.1.

The present disclosure provides a method to improve the stability of a formulation comprising a FGF-21 conjugate disclosed herein, e.g., a PEG-FGF-21 of SEQ ID NO: 5 or SEQ ID NO:6 (e.g., at a concentration of 10 mg/mL or 20 mg/mL), comprising admixing (i) histidine at a concentration of about 20 mM; and (ii) sucrose at a concentration of about 600 mM; wherein the pH is about 7.0.

The present disclosure provides a method to improve the stability of a formulation comprising a FGF-21 conjugate disclosed herein, e.g., a PEG-FGF-21 of SEQ ID NO: 5 or SEQ ID NO:6 (e.g., at a concentration of 10 mg/mL or 20 mg/mL), comprising admixing (i) histidine at a concentration of 20 mM; and (ii) sucrose at a concentration of 600 mM; wherein the pH is 7.0.

The present disclosure provides a method to improve the stability of a formulation comprising a FGF-21 conjugate disclosed herein, e.g., a PEG-FGF-21 of SEQ ID NO: 5 or SEQ ID NO:6, at a concentration of about 10 mg/mL comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 uM; wherein the pH is about 7.1.

The present disclosure provides a method to improve the stability of a formulation comprising a FGF-21 conjugate disclosed herein, e.g., a PEG-FGF-21 of SEQ ID NO: 5 or SEQ ID NO:6, at a concentration of about 20 mg/mL comprising admixing (i) histidine at a concentration of about 20 mM; (ii) sucrose at a concentration of about 600 mM; (iii) Polysorbate 80 at a concentration of about 0.05% (w/v); and (iv) DTPA at a concentration of about 50 uM; wherein the pH is about 7.1.

The present disclosure provides a method to improve the stability of a formulation comprising a FGF-21 conjugate disclosed herein, e.g., a PEG-FGF-21 of SEQ ID NO: 5 or SEQ ID NO:6, at a concentration of about 10 mg/mL comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 uM; wherein the pH is 7.1.

The present disclosure provides a method to improve the stability of a formulation comprising a FGF-21 conjugate disclosed herein, e.g., a PEG-FGF-21 of SEQ ID NO: 5 or SEQ ID NO:6, at a concentration of about 20 mg/mL comprising admixing comprising admixing (i) histidine at a concentration of 20 mM; (ii) sucrose at a concentration of 600 mM; (iii) Polysorbate 80 at a concentration of 0.05% (w/v); and (iv) DTPA at a concentration of 50 uM; wherein the pH is 7.1.

In some embodiments, the concentration of individual excipients included in the pharmaceutical formulation (e.g., DTPA, PS80, histidine, sucrose, or combinations thereof) can be determined/calculated to be the amount (e.g., weight, moles etc.) of the individual excipient added to the pharmaceutical formulation in the course of its manufacture per final volume unit of the finished pharmaceutical formulation. In other embodiments, the concentration of excipients included in the pharmaceutical formulation (e.g., DTPA, PS80, histidine, sucrose, or combinations thereof) is based on the actual amount of the individual excipient in the pharmaceutical formulation.

In some aspects, the methods of manufacture disclosed herein further comprise transferring a formulation disclosed herein, e.g., formulation comprising a FGF-21 conjugate disclosed herein, e.g., a PEG-FGF-21 of SEQ ID NO: 2 or SEQ ID NO:4, at a concentration of about 10 mg or about 20 mg/mL, histidine at a concentration of 20 mM, sucrose at a concentration of 600 mM, Polysorbate 80 at a concentration of 0.05% (w/v), and DTPA at a concentration of 50 uM, wherein the pH is 7.1, to a container. In some aspects, the methods of manufacture disclosed herein further comprise transferring a formulation disclosed herein, e.g., formulation comprising a FGF-21 conjugate disclosed herein, e.g., a PEG-FGF-21 of SEQ ID NO: 5 or SEQ ID NO:6, at a concentration of about 10 mg or about 20 mg/mL, histidine at a concentration of 20 mM, sucrose at a concentration of 600 mM, Polysorbate 80 at a concentration of 0.05% (w/v), and DTPA at a concentration of 50 uM, wherein the pH is 7.1, to a container. In some aspects, the container is a vial. In some aspects, the container is a bag, e.g., a clam shell bag. In some aspects, the bag, e.g., a clam shell bag, has a volume between 6 L and 12 L. In some aspects, the container is a syringe. In some aspects, the syringe is a safety syringe. In some aspects, the syringe is a pre-fillable syringe. In some aspects, the syringe is a BD NEOPAK™ pre-fillable syringe. In some aspects, the container in a self-injection device. In some aspects, about 1 mL of the formulation comprising a FGF-21 conjugate disclosed herein, e.g., a PEG-FGF-21 of SEQ ID NO: 2 or SEQ ID NO:4, at a concentration of about 10 mg or about 20 mg/mL is deposited in the container (e.g., a vial, syringe, or self-injection device). In some aspects, about 1 mL of the formulation comprising a FGF-21 conjugate disclosed herein, e.g., a PEG-FGF-21 of SEQ ID NO: 5 or SEQ ID NO:6, at a concentration of about 10 mg or about 20 mg/mL is deposited in the container (e.g., a vial, syringe, or self-injection device). In some aspects, the container contains multiple doses, e.g., multiple 1 mL doses.

### III. Methods of Treatment

The present disclosure also provides methods of treating or preventing a disease or condition associated with fibrosis and/or diabetes in a subject in need thereof comprising administering to the subject an effective amount of a pharmaceutical formulation comprising a FGF-21 conjugate disclosed herein, e.g., a PEG-FGF-21 of SEQ ID NO: 2 or SEQ ID NO:4.

In some aspects, the disease or condition is diabetes, e.g., type 2 diabetes. In some aspects, the disease or condition is nonalcoholic steatohepatitis (NASH).

In some aspects, a FGF-21 conjugate disclosed herein, e.g., a PEG-FGF-21 of SEQ ID NO: 2 or SEQ ID NO:4, can be used in the methods of treatment or prevention disclosed herein in an amount between about 1 mg and about 40 mg per dose (e.g., 1 mL dose or multiples thereof). In some aspects, the amount of the FGF-21 conjugate for the dose is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)). In some aspects, a FGF-21 conjugate disclosed herein, e.g., a PEG-FGF-21 of SEQ ID NO: 2 or SEQ ID NO:4, can be used in the methods of treatment or prevention disclosed herein in an amount between about 1 mg and about 5 mg per dose, or between about 5 mg and about 10 mg per dose, or between about 10 mg and about 15 mg per dose, or between about 15 mg and about 20 mg per dose, or between about 20 mg and about 25 mg per dose, or between about 25 mg and about 30 mg per dose, or between about 30 mg and about 35 mg per dose, or between about 35 mg and about 40 mg per dose (e.g., 1 mL dose or multiples thereof). In some aspects, a FGF-21 conjugate disclosed herein, e.g., a PEG-FGF-21 of SEQ ID NO: 2 or SEQ ID NO:4, can be used in the methods of treatment or prevention disclosed herein in an amount higher than 40 mg per dose (e.g., 1 mL dose or multiples thereof). In some aspects, the dose is a flat dose.

In some aspects, a FGF-21 conjugate disclosed herein, e.g., a PEG-FGF-21 of SEQ ID NO: 2 or SEQ ID NO:4, can be used in the methods of treatment or prevention disclosed herein in an amount of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg per dose. In a specific aspects, a FGF-21 conjugate disclosed herein, e.g., a PEG-FGF-21 of SEQ ID NO: 2 or SEQ ID NO:4, can be used in the methods of treatment or prevention disclosed herein in an amount of about 10 mg per dose (e.g., 1 mL dose or multiples thereof). In a specific aspects, a FGF-21 conjugate disclosed herein, e.g., a PEG-FGF-21 of SEQ ID NO: 2 or SEQ ID NO:4, can be used in the methods of treatment or prevention disclosed herein in an amount of about 20 mg per dose (e.g., a 1 mL dose or multiples thereof). In some aspects, the dose is a flat dose.

In some aspects, the pharmaceutical formulation is administered subcutaneously, e.g., using a safety syringe or an auto-injector. In some aspects, the pharmaceutical formulation is administered daily or weekly.

In some aspects, administration of an effective amount of the pharmaceutical formulation comprising a FGF-21 conjugate disclosed herein, e.g., a PEG-FGF-21 of SEQ ID NO: 2 or SEQ ID NO:4, to the subject decreases liver stiffness, decreases percentage body fat, decreases body weight, decreases liver-to-body weight ratio, decreases liver lipid content, decreases liver fibrosis area, decreases fasting blood glucose levels, decreases fasting triglyceride levels, decreases LDL cholesterol levels, decreases ApoB levels, decreases ApoC levels, increases HDL cholesterol, or any combination thereof.

In some aspects, the administration of the pharmaceutical formulation comprising a FGF-21 conjugate disclosed herein, e.g., a PEG-FGF-21 of SEQ ID NO: 2 or SEQ ID NO:4, according to the methods of treatment disclosed herein to the subject results in (i) reduction in levels of liver fat; (ii) reduction in levels of liver injury; (iii) reduction in levels of fibrosis; (iv) decrease in levels of fibrosis biomarker serum Pro-C3 (N-terminal type III collagen propeptide); (v) decrease in levels of alanine aminotransferase (ALT); (vi) decrease in levels of aspartate aminotransferase (AST); (vii) increase in levels of serum adiponectin; (viii) decrease in levels of plasma LDL; (ix) increase in levels of plasma HDL; (x) decrease in levels of plasma triglyceride; (xi) reduction in level of liver stiffness; or (xii) any combination thereof, compared to the levels in untreated subjects or to the subject prior to the administration of the pharmaceutical formulation.

The present disclosure provides a method of treating a disease associated with fibrosis comprising administering to a subject in need thereof an effective amount of a pharmaceutical formulation comprising a FGF-21 conjugate disclosed herein, e.g., a PEG-FGF-21 of SEQ ID NO: 2 or SEQ ID NO:4.

In some aspects, the disease associated with fibrosis may affect an organ or tissue such as the pancreas, lung, heart, kidney, liver, eyes, nervous system, bone marrow, lymph nodes, endomyocardium, and/or retroperitoneum. In some aspects, the disease associated with fibrosis may be liver fibrosis or pre-cirrhosis. In some aspects, the disease associated with fibrosis may be selected from: nonalcoholic steatohepatitis (NASH), cirrhosis, diffuse parenchymal lung disease, cystic fibrosis, pulmonary fibrosis, progressive massive fibrosis, idiopathic pulmonary fibrosis, injection fibrosis, renal fibrosis, chronic kidney disease, diabetic kidney disease, focal segmental glomerulosclerosis, membranous nephropathy, IgA nephropathy, myelofibrosis, heart failure, acute heart failure, chronic heart failure, metabolic heart failure, cardiac fibrosis, cataract fibrosis, cataract, ocular scarring, pancreatic fibrosis, skin fibrosis, intestinal fibrosis, intestinal strictures, endomyocardial fibrosis, atrial fibrosis, mediastinal fibrosis, Crohn's disease, retroperitoneal fibrosis, keloid, nephrogenic systemic fibrosis, scleroderma, systemic sclerosis, arthrofibrosis, Peyronie's syndrome, Dupuytren's contracture, diabetic neuropathy, adhesive capsulitis, alcoholic liver disease, hepatosteatosis, viral hepatitis, biliary disease, primary hemochromatosis, drug-related cirrhosis, cryptogenic cirrhosis, Wilson's disease, and, alpha 1-antitrypsin deficiency, interstitial lung disease (ILD), human fibrotic lung disease, liver fibrosis, macular degeneration, retinal retinopathy, vitreal retinopathy, myocardial fibrosis, Grave's ophthalmopathy, drug induced ergotism, cardiovascular disease, atherosclerosis/restenosis, hypertrophic scars, primary or idiopathic myelofibrosis, and inflammatory bowel disease (including, but not limited to, collagenous colitis). In some aspects, the disease associated with fibrosis results from one or more of pulmonary disease, lung cancer, drug therapy, chemotherapy, or radiation therapy. In some aspects, the disease associated with fibrosis results from one or more of aging, heart attack, stroke, myocardial damage, or left ventricular dysfunction. In some aspects, the disease associated with fibrosis may be selected from renal fibrosis, glomerular nephritis, chronic kidney disease, chronic kidney failure, and nephritis associated with systemic lupus, cancer, physical obstructions, toxins, metabolic disease, immunological diseases, or diabetic nephropathy. In some aspects, the disease associated with fibrosis results from one or more of trauma, spinal injury, infection, surgery, ischemic injury, heart attack, burns, environmental pollutant exposure, pneumonia, tuberculosis, or acute respiratory distress syndrome. In some aspects, the disease associated with fibrosis may be selected from pulmonary fibrosis, interstitial lung disease, human fibrotic lung disease, idiopathic pulmonary fibrosis, liver fibrosis, cardiac fibrosis, myocardial fibrosis, macular degeneration, retinal retinopathy, vitreal retinopathy, Grave's ophthalmopathy, drug induced ergotism, cardiovascular disease, atherosclerosis/restenosis, keloids and hypertrophic scars, primary or idiopathic myelofibrosis, inflammatory bowel disease, collagenous colitis, ocular scarring and cataract fibrosis. In some aspects, the disease associated with fibrosis may be selected from NASH, liver fibrosis, and cirrhosis. In some aspects, the disease associated with fibrosis may be NASH. In some aspects, the disease associated with fibrosis may be selected from diabetic kidney disease, chronic kidney disease, and renal fibrosis. In some aspects, the disease associated with fibrosis may be selected from metabolic heart failure and cardiac fibrosis. In some aspects, the disease associated with fibrosis may be lung fibrosis.

In some aspects, the present disclosure provides a method of decreasing the hepatic fat fraction in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4, wherein optionally the subject is at risk of developing or has been diagnosed with NASH.

In some aspects, the present disclosure provides a method of decreasing liver stiffness, decreasing percentage body fat, decreasing body weight, decreasing liver-to-body weight ratio, decreasing liver lipid content, decreasing liver fibrosis area, decreasing fasting blood glucose levels, fasting triglyceride, decreasing LDL cholesterol, decreasing ApoB, decreasing ApoC, and/or increasing HDL cholesterol in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4, wherein optionally the subject is at risk of developing or has been diagnosed with NASH.

In some aspects, the present disclosure provides a method of increasing adiponectin levels in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4, wherein optionally said subject is at risk of developing or has been diagnosed with NASH.

In some aspects, the present disclosure provides a method of treating one or more symptoms associated with NASH in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4.

Provided herein are also methods of treating or preventing NASH in a subject in need thereof, comprising administering to the subject a therapeutically effective amount of a pharmaceutical formulation disclosed herein comprising a variant FGF-21 polypeptide comprising SEQ ID NO: 1, wherein the non-natural p-acetyl-phenylalanine residue thereof is linked via an oxime linkage to a PEG moiety with a molecular weight of about 28 kDa to about 32 kDa. In some aspects, the non-natural p-acetyl-phenylalanine substitutes glutamine 109 of wild type FGF-21 (SEQ ID NO:3). In some aspects, the PEG moiety has a molecular weight of about 30 kDa. In some aspects, the PEG moiety has between about 600 and about 800 ethylene glycol units. In some aspects, the PEG moiety is PEG₆₈₁.

In some specific aspects, the present disclosure provides methods of treating or preventing NASH in a subject in need thereof, comprising administering to the subject a therapeutically effective amount of a pharmaceutical formulation disclosed herein comprising a FGF-21 conjugate of SEQ ID NO: 2 or SEQ ID NO: 4.

In some aspects, the subject may exhibit NASH CRN fibrosis stage 1-3, which optionally is determined by a liver biopsy. In some aspects, prior to treatment the subject may exhibit a fatty liver index of at least about 60. In some aspects, prior to treatment the subject may exhibit a hepatic fat fraction percentage of at least 10%, which optionally is determined by magnetic resonance imaging.

In some aspects, the disclosure provides a method of treating type 1 diabetes or type 2 diabetes in a subject in need thereof, comprising administering to the subject a therapeutically effective amount of a pharmaceutical formulation comprising a FGF-21 conjugate disclosed herein, e.g., a PEG-FGF-21 of SEQ ID NO: 2 or SEQ ID NO:4.

In some aspects, the disclosure provides a method of treating obesity in a subject in need thereof, comprising administering to the subject a therapeutically effective amount of a pharmaceutical formulation comprising a FGF-21 conjugate disclosed herein, e.g., a PEG-FGF-21 of SEQ ID NO: 2 or SEQ ID NO:4.

In some aspects the disclosure provides a method of regulating at least one of glucose and lipid homeostasis, glucose uptake, GLUT 1 expression, and/or serum concentrations of glucose, triglycerides, insulin or glucagon in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4.

In some aspects, the disclosure provides a method of increasing insulin sensitivity, increasing levels of adiponectin, reducing levels of blood glucose, reducing levels of glucagon, reducing levels of triglyceride, reducing levels of fructosamine, reducing levels of low density cholesterol, or reducing levels of C-reactive protein in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4.

In some aspects the disclosure provides a method of treating a condition or disorder selected from obesity, diabetes, pancreatitis, insulin resistance, hyperinsulinemia, glucose intolerance, hyperglycemia, metabolic syndrome, impaired glucose tolerance, inadequate glucose clearance, high blood glucose, and Prader-Willi syndrome in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4.

In some aspects the disclosure provides a method of treating an insulin related condition or disorder selected from Type A Insulin Resistance, Type C Insulin Resistance (AKA HAIR-AN Syndrome), Rabson-Mendenhall Syndrome, Donohue's Syndrome or Leprechaunism, hyperandrogenism, hirsuitism, or acanthosis nigricans in a subject in need thereof, comprising administering to the subject an effective amount of a pharmaceutical formulation comprising a FGF-21 conjugate disclosed herein, e.g., a PEG-FGF-21 of SEQ ID NO: 2 or SEQ ID NO:4.

In some aspects, the pharmaceutical formulations comprising a FGF-21 conjugate disclosed herein, e.g., a PEG-FGF-21 of SEQ ID NO: 2 or SEQ ID NO:4, can be administered or via injection. In some aspects, the pharmaceutical formulations comprising a FGF-21 conjugate disclosed herein, e.g., a PEG-FGF-21 of SEQ ID NO: 2 or SEQ ID NO:4, can be administered via subcutaneous injection, IV injection, intraperitoneal injection, or intramuscular injection, e.g., using a syringe (such as a safety syringe) or an auto-injector.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per day, or less frequently than about once per day. In some aspects, the pharmaceutical formulations comprising a FGF-21 conjugate disclosed herein, e.g., a PEG-FGF-21 of SEQ ID NO: 2 or SEQ ID NO:4, can be administered at a frequency of about twice per week, or less frequently than about twice per week. In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per week, or less frequently than about twice per week. In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per two weeks, or less frequently than about twice per week.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per three weeks, or less frequently than about twice per week.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per month, or less frequently than about once per month.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of once per four weeks.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per day.

In some aspects, the pharmaceutical formulations comprising a FGF-21 conjugate disclosed herein, e.g., a PEG-FGF-21 of SEQ ID NO: 2 or SEQ ID NO:4, can be administered at a frequency of about once per week.

In some aspects, the pharmaceutical formulations comprising a FGF-21 conjugate disclosed herein, e.g., a PEG-FGF-21 of SEQ ID NO: 2 or SEQ ID NO:4, can be administered in an amount selected from about 1 mg, about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95, and about 100 mg of FGF-21 conjugate disclosed herein, per dose. In a specific aspects, the pharmaceutical formulations comprising a FGF-21 conjugate disclosed herein, e.g., a PEG-FGF-21 of SEQ ID NO: 2 or SEQ ID NO:4, can be administered in an amount of about 10 mg/dose (e.g., 1 mL doses or multiples thereof). In a specific aspects, the pharmaceutical formulations comprising a FGF-21 conjugate disclosed herein, e.g., a PEG-FGF-21 of SEQ ID NO: 2 or SEQ ID NO:4, can be administered in an amount of about 20 mg/dose (e.g., 1 mL doses or multiples thereof). In some aspects, the dose is a flat dose.

For example, a pharmaceutical formulation comprising a FGF-21 conjugate disclosed herein, e.g., a PEG-FGF-21 of SEQ ID NO: 2 or SEQ ID NO:4, herein can be administered to a subject at a concentration of between about 0.1 and 100 mg/kg of body weight of recipient subject. In some aspects, a pharmaceutical formulation comprising a FGF-21 conjugate disclosed herein, e.g., a PEG-FGF-21 of SEQ ID NO: 2 or SEQ ID NO:4, can be administered to a subject at a concentration of FGF-21 conjugate disclosed herein, e.g., a PEG-FGF-21 of SEQ ID NO: 2 or SEQ ID NO:4, of about 0.5-5 mg/kg of body weight of recipient subject. In another aspect, a pharmaceutical formulation comprising a FGF-21 conjugate disclosed herein, e.g., a PEG-FGF-21 of SEQ ID NO: 2 or SEQ ID NO:4, can be administered to a recipient subject with a frequency of between once per day and once per two weeks, such as about once or twice per week, once every two days, once every three days, once every four days, once every five days, or once every six days.

Pharmaceutical formulations of the present disclosure, i.e., formulation comprising a FGF-21 conjugate disclosed herein, e.g., a PEG-FGF-21 of SEQ ID NO: 2 or SEQ ID NO:4, can be administered by any conventional route suitable for proteins or peptides, including, but not limited to parenterally, e.g., injections including, but not limited to, subcutaneously or intravenously or any other form of injections or infusions. In some aspects, the pharmaceutical formulation can be administered using a syringe, e.g., a safety syringe. In some aspects, the pharmaceutical formulation can be administered using an auto-injector.

### IV. Articles of Manufacture and Kits

The present disclosure also provides an article or manufacture or kit comprising (i) a FGF-21 pharmaceutical formulation comprising a FGF-21 conjugate disclosed herein, e.g., a PEG-FGF-21 of SEQ ID NO: 2 or SEQ ID NO:4, and (ii) instructions for use. In some aspects, the article of manufacture or kit can comprise a container. Suitable containers include, for example, bottles, vials, syringes and test tubes. In some aspects, the container may be formed from a variety of materials such as glass, plastic or metals. In some aspects, the container holds a pharmaceutical formulation, e.g., a liquid formulation, comprising a FGF-21 conjugate disclosed herein, e.g., a PEG-FGF-21 of SEQ ID NO: 2 or SEQ ID NO:4. In some aspects, the container holds a pharmaceutical formulation, e.g., a liquid formulation, comprising a FGF-21 conjugate disclosed herein, e.g., a PEG-FGF-21 of SEQ ID NO: 5 or SEQ ID NO:6.

The label on, or associated with, the container may indicate directions for reconstitution and/or use. For example, the label may indicate that the pharmaceutical formulation comprising a FGF-21 conjugate disclosed herein, e.g., a PEG-FGF-21 of SEQ ID NO: 2 or SEQ ID NO:4, is to be diluted to protein concentrations as described above. The label may further indicate that the formulation is a subcutaneous formulation useful or intended for subcutaneous administration.

In some aspects, the container holding the formulation may be a multi-use vial, which allows for repeat administrations (e.g., from 2 to 6 administrations, or more) of, for example, the subcutaneous formulation. Alternatively, the container may be a pre-filled syringe containing, for example, the subcutaneous formulation.

The article of manufacture or kit may further comprise a second container comprising, for example, a solvent. The article of manufacture may further include other materials desirable from a commercial and user standpoint, including other buffers, diluents, filters, needles, syringes, and package inserts with instructions for use. In some aspects, the kit or article of manufacture comprises a syringe (e.g., a safety syringe). In some aspects, the kit or article of manufacture comprises an auto-injector.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

### EXAMPLES

### Example 1

### PEG-FGF-21 Formulation Development

For initial studies, PEG-FGF-21 was formulated at 7.5 mg/mL in 20 mM Tris, 250 mM sucrose, pH 8.3. High deamidation rates observed in this formulation made it necessary to store the drug product frozen at −20° C. Therefore, additional work was performed to develop a ready-to use (RTU) formulation allowing 2-8° C. storage of the drug product.

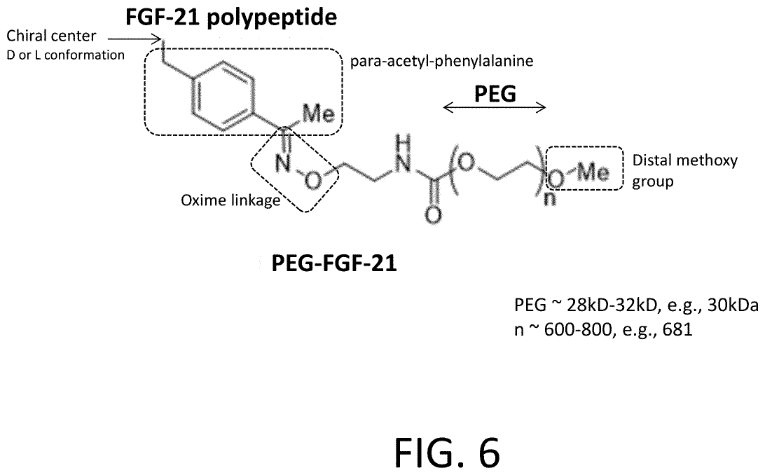

PEG-FGF-21 (SEQ ID NO: 2) was made by attaching a linear 30-kDa PEG moiety comprising a distal methoxy group as depicted in FIG. 6 to a variant FGF-21 (SEQ ID NO: 1) in a site-specific manner, resulting in the molecular conjugate structure shown in FIG. 6, wherein the para-acetyl-phenylalanine has the L conformation. To achieve site-specific PEGylation, activated PEG was reacted by methods known in the art with the non-natural amino acid, para-acetyl-L-phenylalanine (pAF), in position 109 of the FGF-21 molecule to form a chemically stable oxime-linked PEG-protein conjugate. Activated PEG is commercially available from many sources, including for example from Nippon Oil & Fats Co., Ltd., Tokyo, Japan.

Lower formulation pH was shown to increase aggregation rates of the molecule, while higher formulation pH was shown to increase deamidation rates. At the target protein concentration of 10 mg/mL, pH 7.0 was found to be the best balance between aggregation and deamidation rates. Increasing sucrose concentration was shown to further stabilize the molecule against aggregation.

The formulation chosen for further studies was 10 mg/mL PEG-FGF-21 in 20 mM L-histidine, 600 mM sucrose, pH 7.0. Given a contemplated weekly dose of 20 mg per patient, a higher concentration drug product in a pre-filled syringe was developed. To enable the higher concentration drug product, further optimization of the formulation was necessary.

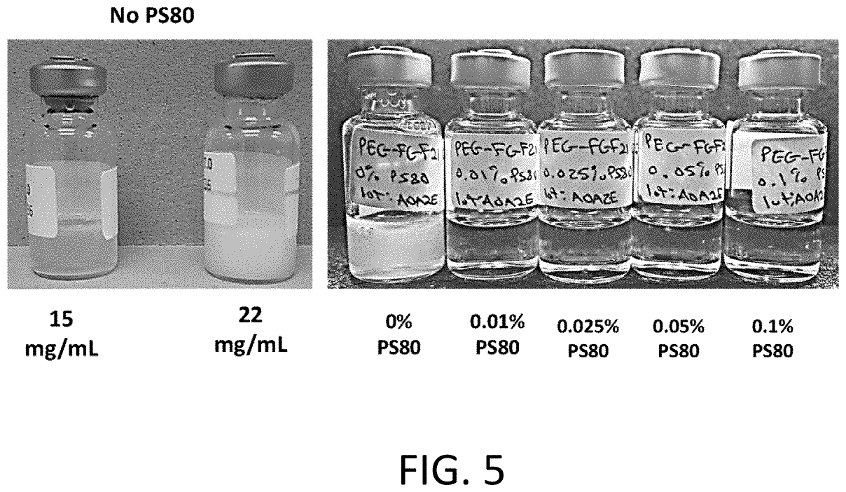

Initial development of a higher concentration formulation was conducted in 3 mL type I glass vials. Upon agitation of 15 mg/mL and 22 mg/mL PEG-FGF-21 in 20 mM L-histidine, 600 mM sucrose, pH 7.0 air bubble entrapment was observed and the solutions became increasingly turbid (FIG. 5, left images). In an attempt to mitigate the issue, polysorbate 80 was used. Polysorbate 80 was able to prevent air bubble entrapment (FIG. 5, right images). Therefore, polysorbate 80 was added at a concentration of 0.05% (w/v) to the high concentration formulation of PEG-FGF-21.

Oxidation is a critical quality attribute of PEG-FGF-21 and oxidation of methionine-169 near the C-terminus abolishes receptor binding and thus activity of the molecule. Metal catalyzed oxidation is one of the mechanisms by which protein can be oxidized. Trace concentrations of metals can be present in protein formulation due to carry over from cell culture, contact with stainless steel vessels, impurities in excipients, formation process of pre-fillable syringes, etc.

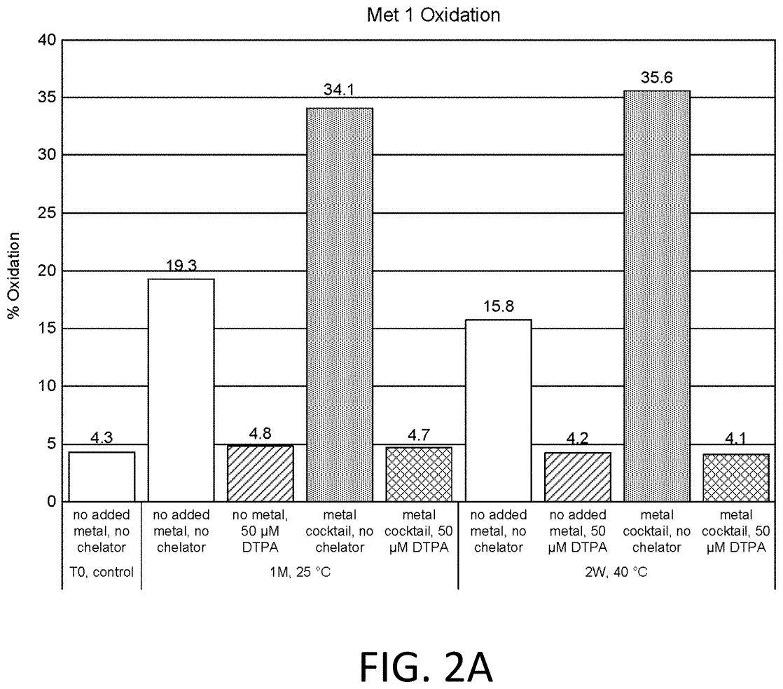

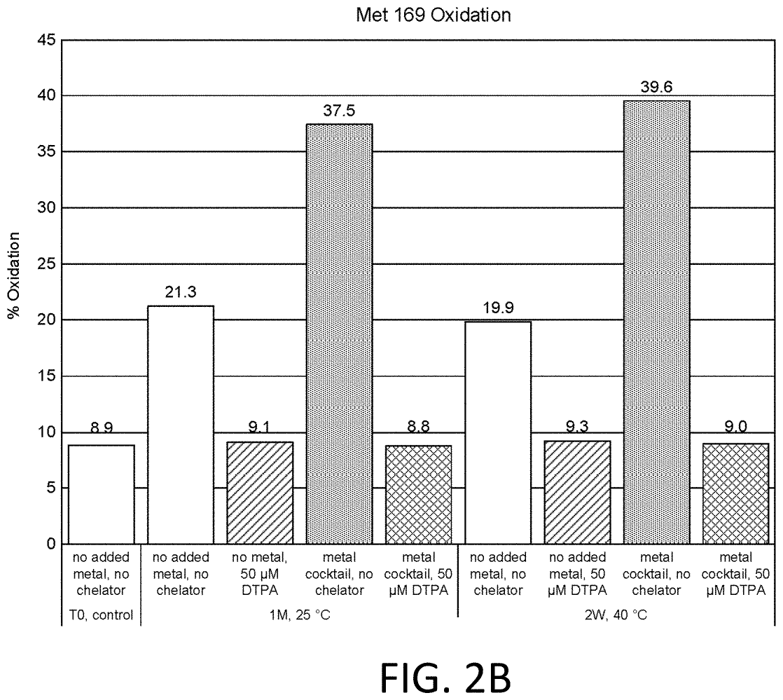

To test if PEG-FGF-21 was sensitive to metal catalyzed oxidation, samples with and without addition of a metal cocktail (250 ppm Fe, 10 ppm Cu, 15 ppm Cr, 15 ppm Ni, 10 ppm Mo) in the presence or absence of 0.05 mM pentetic acid (diethylenetriaminepentaacetic acid, DTPA) were incubated for one month at 25° C. and for two weeks at 40° C., respectively, and oxidation levels of methionine 1 and methionine 169 were determined by tryptic peptide mapping (FIGS. 2A and 2B).

In the absence of pentetic acid (DTPA), significant oxidation of both methionine residues was observed even in samples without added metal while in samples spiked with a metal cocktail oxidation levels were even higher. In samples with 50 μM pentetic acid, no increase in oxidation levels were observed for either methionine. This suggests that PEG-FGF-21 is sensitive to metal catalyzed oxidation and that there are already trace levels of metals present in the formulation. Pentetic acid effectively chelated the metals present in the formulation as well as additional metals spiked into the formulation and prevented metal catalyzed oxidation of methionines 1 and 169. Therefore, 50 μM pentetic acid was added to the high concentration formulation of PEG-FGF-21.

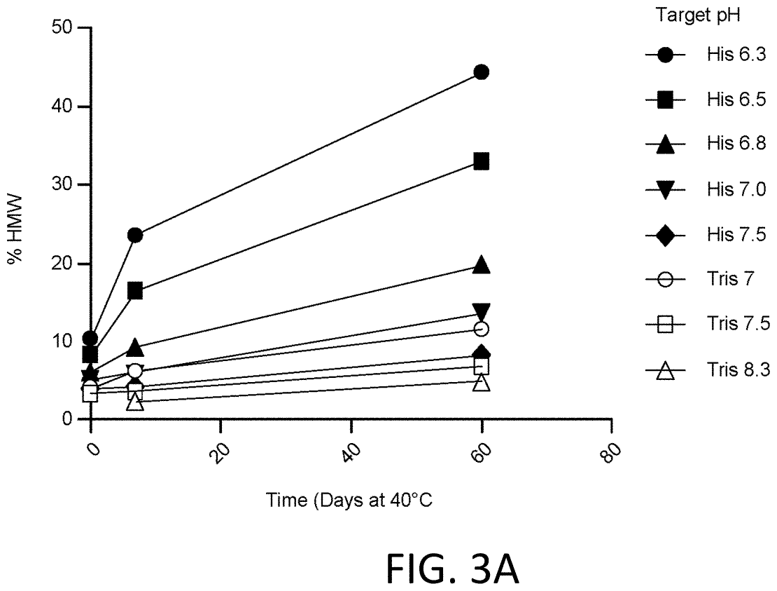

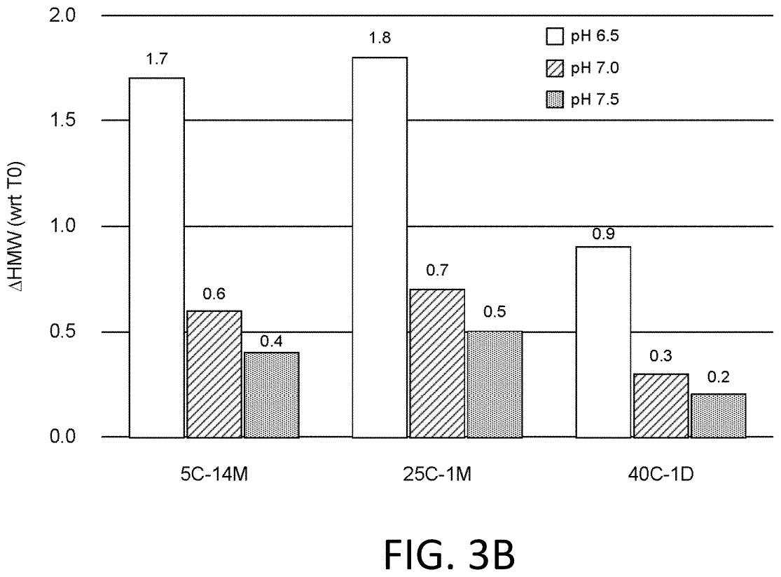

To optimize the pH for the higher concentration formulation, we reviewed aggregation data of PEG-FGF-21 collected between pH 6.3 and 8.5 showing that aggregation rates rapidly increased below pH 6.8 (FIGS. 3A and 3B). Therefore, we increased the target pH for the high concentration formulation from pH 7.0 to pH 7.1

To compare the new high concentration formulation to the previous formulation, PEG-FGF-21 at 20 mg/mL in 20 mM L-histidine, 600 mM sucrose, 0.05 mM pentetic acid, 0.05% (w/v) polysorbate 80, pH 7.0 (prior to pH optimization) was placed on stability and compared to PEG-FGF-21 at 22.5 mg/mL in 20 mM L-histidine, 600 mM sucrose, pH 7.0.

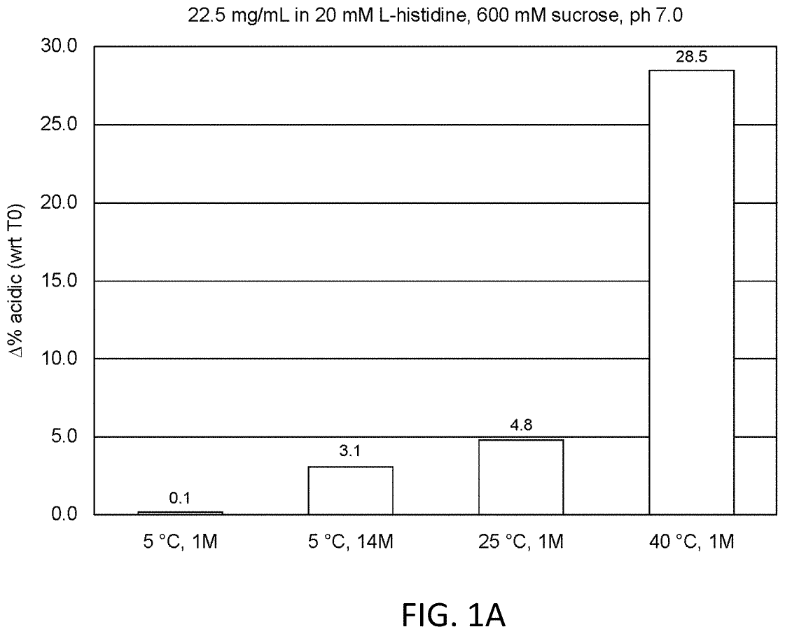

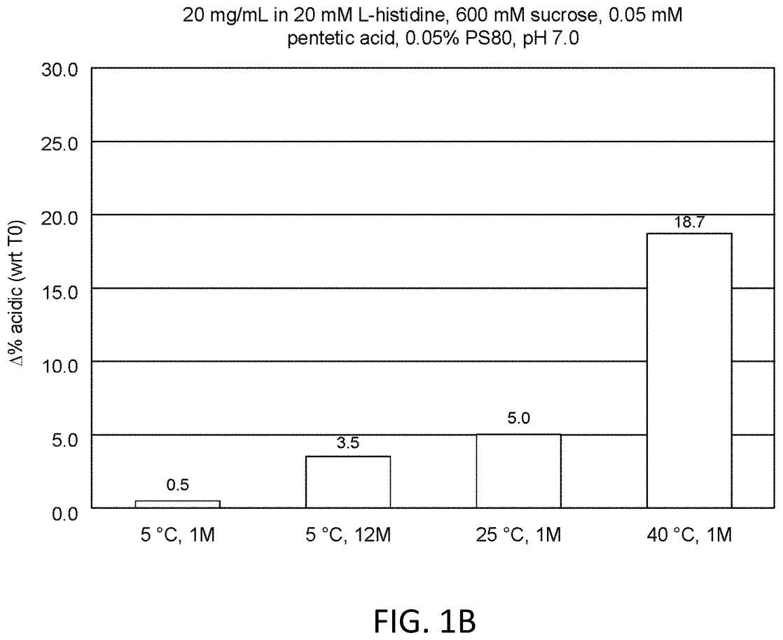

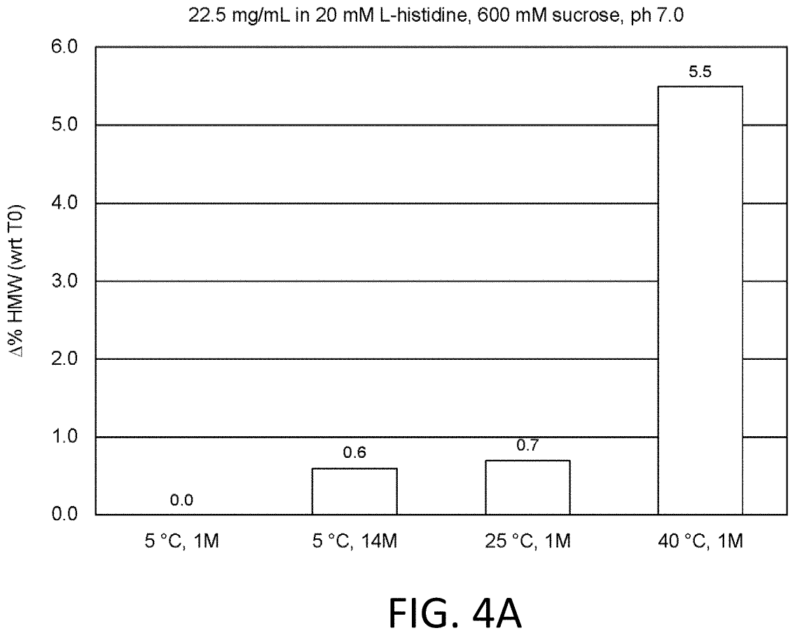

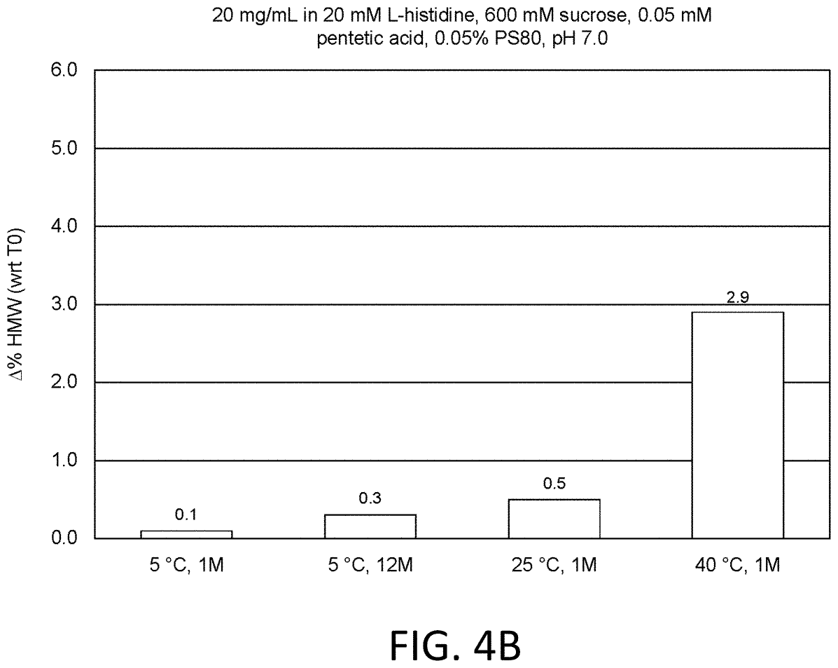

As shown in FIGS. 4A and 4B, FIGS. 1A and 1B, addition of polysorbate 80 and pentetic acid to the PEG-FGF-21 formulation resulted in lower aggregation rates at all temperatures and less deamidation at 40° C.

### Test Methods

Aggregation: Aggregation was tested by size exclusion high performance liquid chromatography on a commercially available system (e.g., Agilent Technologies 1100 Series HPLC system with PDA detector or Waters Alliance e2695 Series HPLC with PDA detector) fitted with a commercially available analytical column (Tosoh TSK gel G3000SWXL, 7.8×300 mm, P/N: 08541). A mobile phase of 95% phosphate buffered saline (PBS) and 5% ethanol at a flow rate of 0.8 ml/min was used to separate high molecular weight species from protein monomer. Samples were diluted to a protein concentration of 0.5 mg/mL with PBS and 0.01 mg (0.02 mL) were injected for each experiment. The amount of aggregate was determined by dividing the area of high molecular weight peaks by the total area of all observed peaks using instrument software.

Deamidation: Deamidation was tested by anion exchange high performance liquid chromatography on a commercially available system (e.g., Agilent Technologies 1100 Series HPLC system with PDA detector or Waters Alliance e2695 Series HPLC with PDA detector) fitted with a commercially available analytical column (Agilent Bio WAX, non-porous, 5 μm column, 4.6×250 mm, P/N: 5190-2487). Mobile phase A consisted of 20 mM Tris, pH 8.2 and mobile phase B consisted of 20 mM Tris, 500 mM Sodium Chloride, pH 8.2. A linear gradient from 2% B to 67% B over 20 minutes at a flow rate of 1.0 ml/min was used to separate charged protein variants. Samples were diluted to a protein concentration of 1 mg/mL with mobile phase A and 0.075 mg (0.075 mL) were injected for each experiment. The amount of deamidation was determined by dividing the area of acidic variant peaks by the total area of all observed peaks using instrument software.

Oxidation: Oxidation was tested by tryptic peptide mapping. After protein samples were digested with trypsin, resulting peptides were separated by reverse phase ultra-high performance liquid chromatography on a commercially available system (e.g., Waters Acquity UPLC) fitted with a commercially available analytical column (Waters Acquity C18 BEH peptide RP UPLC column, 2.1×150 mm, 1.7-μm particle size, 130-Å pore size P/N: 186003556). Mobile phase A consisted of 0.2% trifluoroacetic acid (TFA) in water and mobile phase B consisted of 0.2% TFA in acetonitrile. A complex gradient from 10% B to 40% B over 27 minutes at a temperature of 60° C. and a flow rate of 0.3 ml/min was used to separate tryptic peptides. The amount of oxidation was determined by dividing the area of oxidized peptide peaks by the total area of oxidized and corresponding non-oxidized peaks using instrument software.

Particulate Formation and Air Bubble Formation: Visual Observation.

Concentration of PEG-FGF-21: The concentration of PEG-FGF-21 in the pharmaceutical formulations disclosed herein was measured by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N OC0009-1)), at 280 nm using an Extinction Coefficient of 0.87 (mL/(mg*cm)). PEG-FGF-21 dose amounts throughout the present disclosure are based on the pharmaceutical formulations measured in this way. Slope Spectroscopy is also applicable to any FGF-21 conjugates generally, not only PEG-FGF-21.

Determination of pH: pH was determined according to standard methods (USP<791>).

Concentration of excipients: The concentration of individual excipients in the pharmaceutical formulation disclosed herein (e.g., DTPA, PS80, Histidine, Sucrose) was determined/calculated to be the amount (weight, moles etc.) of the individual excipient added to the pharmaceutical formulation in the course of its manufacture per final volume unit of the finished pharmaceutical formulation. Alternatively, the concentration of excipients can be based on the actual amount of the individual excipient in the pharmaceutical formulation.

1-111. (canceled)

112. A pharmaceutical formulation comprising
(i) a fibroblast growth factor 21 (FGF-21) polypeptide comprising an amino acid sequence having at least about 80%, at least about 85%, at least about 90%, at least about 95%, at least about 96%, at least about 97%, at least about 98%, or at least about 99% amino acid sequence identity to the amino acid sequence of SEQ ID NO: 3, conjugated to a polyethylene glycol (PEG) moiety via the side chain of an amino acid in the amino acid sequence (“FGF-21 conjugate”); and,
(ii) an aminopolycarboxylic acid cation chelator comprising diethylenetriaminepentaacetic acid (DTPA),
wherein the formulation has improved stability compared to a reference formulation that does not contain the aminopolycarboxylic acid cation chelator,
wherein the FGF-21 conjugate comprises formula I: 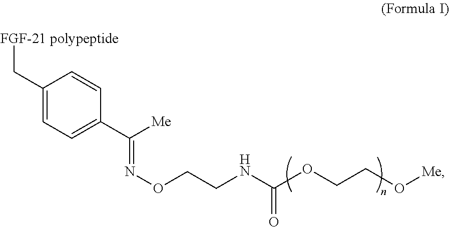
wherein n is an integer from about 500 to about 900, and
wherein the FGF-21 conjugate is present in an amount between about 1 mg and about 40 mg per unit dose.

113. The pharmaceutical formulation of claim 112, wherein the FGF-21 conjugate corresponds to a compound of SEQ ID NO: 2, 4, 5 or 6.

114. The pharmaceutical formulation of claim 112, wherein the FGF-21 polypeptide conjugate is in an L conformation.

115. The pharmaceutical formulation of claim 112, wherein the FGF-21 conjugate is present at a concentration between about 1 mg/ml and about 40 mg/ml, or between about 10 mg/ml or about 20 mg/ml, as measured by Slope Spectroscopy at 280 nm using an extinction coefficient of 0.87 mL/(mg*cm).

116. The pharmaceutical formulation of claim 112, wherein the FGF-21 conjugate is present in an amount of about 1 mg per unit dose, about 5 mg per unit dose, about 10 mg per unit dose, about 20 mg per unit dose, or about 40 mg per unit dose.

117. The pharmaceutical formulation of claim 112, which exhibits one or more of:
(a) a lower rate of polypeptide deamidation when stored at 40° C. for about a month with respect to the reference formulation;
(b) a lower rate of high molecular weight (HMW) polypeptide aggregation when stored at 40° C. for about a month with respect to the reference formulation;
(c) reduced oxidation of one or more methionines selected from Met1 and/or Met169 of the FGF-21 polypeptide;
(d) reduced methionine oxidation at 25° C. and/or 40° C.; or.
(c) any combination thereof.

118. The pharmaceutical formulation of claim 112, wherein the DTPA cation chelator is present in an amount between about 25 μM and about 75 μM DTPA.

119. The pharmaceutical formulation of claim 112, wherein the DTPA cation chelator is present in an amount of about 40 μM, about 45 μM, about 50 μM, about 55 μM, or about 60 μM DTPA.

120. The pharmaceutical formulation of claim 112, wherein the pharmaceutical formulation further comprises
(a) an amino acid buffering agent;
(b) an osmotic regulator;
(b) a nonanionic surfactant; or
(d) a combination thereof.

121. The pharmaceutical formulation of claim 120, wherein the amino acid buffering agent comprises histidine.

122. The pharmaceutical formulation of claim 121, wherein the histidine buffering agent is present in an amount of about 10 mM to about 100 mM histidine.

123. The pharmaceutical formulation of claim 120, wherein the osmotic regulator comprises sucrose.

124. The pharmaceutical formulation of claim 123, wherein the sucrose is present in an amount of about 100 mM to about 1 M sucrose.

125. The pharmaceutical formulation of claim 120, wherein the nonanionic surfactant comprises polyoxyethylene (20) sorbitan monooleate (polysorbate 80).

126. The pharmaceutical formulation of claim 125, wherein the polysorbate 80 surfactant is present in an amount of about 0.01% to about 0.1% (w/v).

127. The pharmaceutical formulation of claim 112, wherein the formulation is an aqueous formulation.

128. A pharmaceutical composition comprising:
(i) a FGF-21 conjugate corresponding to a compound of SEQ ID NO: 2, 4, 5 or 6 at a concentration of 20 mg/mL as measured by Slope Spectroscopy at 280 nm using an extinction coefficient of 0.87 mL/(mg*cm);
(ii) histidine at a concentration between about 10 mM and about 50 mM;
(iii) sucrose at a concentration between about 100 mM and about 1M;
(iv) Polysorbate 80 at a concentration between about 0.01% and about 0.1% (w/v); and,
(v) DTPA at a concentration between about 10 μM and about 100 μM;
wherein the pH of the formulation is between about 6.7 and about 7.5, and
wherein the FGF-21 conjugate is present in an amount between about 1 mg and about 40 mg per unit dose.

129. A pharmaceutical formulation comprising:
(i) a FGF-21 conjugate corresponding to a compound of SEQ ID NO: 2, 4, 5 or 6 at a concentration of 20 mg/mL as measured by Slope Spectroscopy at 280 nm using an extinction coefficient of 0.87 mL/(mg*cm);
(ii) histidine at a concentration of 20 mM;
(iii) sucrose at a concentration of 600 mM;
(iv) Polysorbate 80 at a concentration of 0.05% (w/v); and
(v) DTPA at a concentration of 50 uM;
wherein the pH is 7.1,
wherein the FGF-21 conjugate is present in an amount between about 1 mg and about 40 mg per unit dose.

130. The pharmaceutical composition of claim 129 comprising FGF-21 conjugate in an amount of about 20 mg or about 40 mg per unit dose.

131. A vial comprising:
(i) a FGF-21 conjugate corresponding to a compound of SEQ ID NO: 2, 4, 5 or 6 at a concentration of 20 mg/mL as measured by Slope Spectroscopy at 280 nm using an extinction coefficient of 0.87 mL/(mg*cm);
(ii) histidine at a concentration between about 10 mM and about 50 mM;
(iii) sucrose at a concentration between about 100 mM and about 1M;
(iv) Polysorbate 80 at a concentration between about 0.01% and about 0.1% (w/v); and,
(v) DTPA at a concentration between about 10 μM and about 100 μM;
wherein the pH is between about 6.7 and about 7.5, and
wherein the FGF-21 conjugate is present in an amount between about 1 mg and about 40 mg per unit dose.

132. A vial comprising:
(i) a FGF-21 conjugate corresponding to a compound of SEQ ID NO: 2, 4, 5 or 6 at a concentration of 20 mg/mL as measured by Slope Spectroscopy at 280 nm using an extinction coefficient of 0.87 mL/(mg*cm);
(ii) histidine at a concentration of 20 mM;
(iii) sucrose at a concentration of 600 mM;
(iv) Polysorbate 80 at a concentration of 0.05% (w/v); and
(v) DTPA at a concentration of 50 uM;
wherein the pH is 7.1, and
wherein the FGF-21 conjugate is present in an amount between about 1 mg and about 40 mg per unit dose.

133. The vial of claim 132, comprising FGF-21 conjugate in an amount of about 5 mg, about 10 mg, about 20 mg or about 40 mg per vial.

134. A kit or article of manufacture comprising (i) the pharmaceutical formulation of claim 112, and (ii) instructions for use.

135. A kit or article of manufacture comprising (i) the vial of claim 131, and (ii) instructions for use.

136. A method of treating or preventing a disease or condition associated with fibrosis and/or diabetes in a subject in need thereof comprising administering to the subject an effective amount of a pharmaceutical formulation of claim 112.

137. The method of claim 136, wherein the disease or condition is type 2 diabetes or nonalcoholic steatohepatitis (NASH).

138. The method of claim 136, wherein the pharmaceutical formulation is administered subcutaneously using a safety syringe.

139. The method of claim 136, wherein the pharmaceutical formulation is administered daily or weekly.


SEQUENCE LISTING (6 sequences)

SEQ ID NO: 1 - protein, 182 aa, Artificial Sequence
  Q109pAF FGF-21
  misc_feature (109)..(109): Xaa is pAF (para-acetyl-L-phenylalanine)
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLPGNKSPHRDPAPRGPARFLPLPGLPPAPPEPPGILAPQPPDVGSSDPLSMVGPSQGRSPSYAS

SEQ ID NO: 2 - protein, 182 aa, Artificial Sequence; identical to SEQ ID NO: 1
  Q109pAF FGF-21, PEGylated
  misc_feature (109)..(109): Xaa is pAF (para-acetyl-L-phenylalanine). Poly(ethylene glycol) linked to pAF via oxime linkage

SEQ ID NO: 3 - protein, 182 aa, Artificial Sequence
  FGF-21 wild type sequence
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 4 - protein, 182 aa, Artificial Sequence; identical to SEQ ID NO: 1
  Q109pAF FGF-21, PEGylated with 681 unit PEG (PEG681)
  misc_feature (109)..(109): Xaa is pAF (para-acetyl-L-phenylalanine).pAF is linked to PEG681 (linear PEG comprising 681ethylene glycol units)via an oxime linkage, e.g.,formed by reaction between the acetyl group of pAF and the aminooxy group of the 30kDa methyl PEG681 aminooxy

SEQ ID NO: 5 - protein, 181 aa, Artificial Sequence
  Q109pAF FGF-21, PEGylated, no N-term Met
  misc_feature (108)..(108): Xaa is pAF (para-acetyl-L-phenylalanine). pAF is linked to 28-32 kDA PEG via an oxime linkage
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLPGNKSPHRDPAPRGPARFLPLPGLPPAPPEPPGILAPQPPDVGSSDPLSMVGPSQGRSPSYAS

SEQ ID NO: 6 - protein, 181 aa, Artificial Sequence; identical to SEQ ID NO: 5
  Q109pAF FGF-21, PEGylated (PEG is PEG681), no N-term Met
  Misc_feature (108)..(108): Xaa is pAF(para-acetyl-L-phenylalanine).pAF is linked to PEG681 (linear PEG comprising 681  ethylene glycol units) via an oxime linkage, e.g.,formed by reaction between the acetyl group of pAF and the aminooxy group of the 30kDa methyl PEG681 aminooxy